(19) United States
(12) Patent Application Publication
(10) Pub. No.: US 2025/0306033 A1
(43) Pub. Date: Oct. 2, 2025

(54) INDIVIDUALIZED LIFESTYLE INTERVENTION FOR OBESITY MANAGEMENT BASED ON OBESITY PHENOTYPES

(71) Applicant: Mayo Foundation for Medical Education and Research, Rochester, MN (US)

(72) Inventors: Andres J. ACOSTA, Rochester, MN (US); Michael L. CAMILLERI, Rochester, MN (US); Donald HENSRUD, Rochester, MN (US); Matthew CLARK, Rochester, MN (US); Lizeth CIFUENTES, Rochester, MN (US)

(21) Appl. No.: 18/862,995

(22) PCT Filed: May 10, 2023
(86) PCT No.: PCT/US2023/066809

§ 371 Date: Nov. 5, 2024

Related U.S. Application Data
(60) Provisional application No. 63/340,111, filed on May 10, 2022.

(51) Int. Cl. G01N 33/68; A61K 31/137; A61K 31/7048; A61K 38/26; A61P 3/04; C12Q 1/6883

(52) U.S. Cl. CPC G01N 33/6893; A61K 31/137; A61K 31/7048; A61K 38/26; A61P 3/04; C12Q 1/6883; C12Q 2600/156; G01N 2800/044; G01N 2800/52

(57) ABSTRACT

The present disclosure relates to methods and materials for assessing and/or treating obese mammals (e.g., obese humans). For example, methods and materials for using one or more interventions (e.g., individualized dietary interventions alone or in combination with pharmacological intervention(s)) to treat obesity and/or obesity-related comorbidities in a mammal (e.g., a human) identified as being likely to respond to a particular intervention (e.g., individualized dietary interventions alone or in combination with pharmacological intervention(s)) are provided.

### CROSS-REFERENCE TO RELATED APPLICATIONS

This application claims the benefit of priority to U.S. Provisional Application No. 63/340,111, filed May 10, 2022, which is hereby incorporated by reference in its entirety for all purposes.

### FIELD

The present disclosure relates to methods and materials for treating obesity in a subject (e.g., humans) following assessment of the subject's obesity phenotype. For example, this document provides methods and materials for treating a subject following diagnosing the subject's obesity phenotype via use of an obesity analyte signature of the subject. For example, this document provides methods for providing dietary advice for use in treating obesity and/or obesity-related comorbidities in a mammal (e.g., a human) identified as being likely to respond to particular dietary advice such that the dietary advice can be used alone or in combination with one or more interventions (e.g., one or more pharmacological interventions, exercise and physical activity, behavior therapy, etc.) to treat obesity and/or obesity-related comorbidities.

### BACKGROUND

Obesity is a chronic, relapsing, multifactorial disease (Acosta et al., Clin. Gastroenterol. Hepatol., 15(5):631-49 e10 (2017); and Heymsfield et al., N. Engl. J. Med., 376(15):1492 (2017)), whose prevalence continues to increase worldwide (Ng et al., Lancet, 384(9945):P766-781 (2014); Collaborators GO, N. Engl. J. Med., 377:13-27 (2017); and Flegal et al., JAMA, 307(5):491-7 (2012)). In the United States alone, 69% of adults are overweight or obese (Flegal et al., 2012 JAMA 307:491-497). Estimated costs to the healthcare system are more than $550 billion annually. Increased severity of obesity correlates with a higher prevalence of the associated co-morbidities. Likewise, obesity increases the risk of premature mortality (Hensrud et al., 2006 Mayo Clinic Proceedings 81(10 Suppl):S5-10). Obesity affects almost every organ system in the body and increases the risk of numerous diseases including type 2 diabetes mellitus, hypertension, dyslipidemia, cardiovascular disease, and cancer. It is estimated that a man in his twenties with a BMI over 45 will have a 22% reduction (13 years) in life expectancy (Fontaine, K R et al., JAMA, 289(2), 187-193 (2003)).

The complexities of obesity result in redundant and adaptive mechanisms to preserve energy; consequently, obesity is a remarkably heterogeneous disease, and sustained, successful outcomes with current treatment paradigms remain a challenge in clinical practice (Loos et al., Cell. Metab., 25(3):535-43 (2017); and MacLean et al., Obesity, 25 Suppl 1:S8-S16 (2017)). The heterogeneity among patients with obesity is particularly apparent in the treatment response to obesity interventions, such as diets, medications, devices, and surgery. Irrespective of the intervention, treatment response in highly variable; 30% of patients are poor responders (total body weight loss <5%), while 30% are regarded as positive responders, achieving clinically significant total body weight loss (>10%) (Heymsfield et al., N. Engl. J. Med., 376(15):1492 (2017)). Despite considerable attempts to address predictors for weight loss, little is currently known about the predictors of response to obesity interventions (Loos et al., Cell. Metab., 25(3):535-43 (2017)).

Accordingly, there is an unmet need in the art to match subjects suffering from obesity to interventions most likely to produce an efficacious response (e.g., sustained weight loss) in a particular obese subject. The materials and methods provided herein address this need.

### SUMMARY

In one aspect, provided herein is a method of treating obesity in a subject in need thereof, the method comprising: (a) identifying an obesity phenotype of a subject by detecting an intervention responsive obesity analyte signature in a sample obtained from a subject; and (b) administering a predetermined lifestyle and behavioral plan to the subject, wherein the predetermined lifestyle and behavioral plan is individualized to the obesity phenotype of the subject, thereby treating obesity in the subject. In some cases, the administration of the predetermined diet results in weight loss, wherein at least part of the weight loss is due to a loss of fat mass. In some cases, the administration of the predetermined diet is effective to reduce the total body weight of the subject by at least 4%. In some cases, the administration of the predetermined diet is effective to reduce the total body weight of the subject by 2% to 8%. In some cases, the administration of the predetermined diet is effective to reduce the waist circumference of the subject by from about 1 inches to about 10 inches. In some cases, determining the obesity phenotype of the subject further comprises obtaining results from a Hospital Anxiety and Depression Scale (HADS) questionnaire. In some cases, the detecting the intervention responsive obesity analyte signature comprises detecting the presence, absence, or level of one or more metabolites, detecting the presence, absence, or level of one or more peptides, and/or detecting the presence, absence, or level of one or more single nucleotide polymorphisms (SNPs) in the sample obtained from the subject. In some cases, the sample is selected from the group consisting of a blood sample, a saliva sample, a urine sample, a breath sample, and a stool sample. In some cases, the obesity analyte signature comprises 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, ghrelin, peptide tyrosine tyrosine (PYY), GLP-1 and phenylalanine. In some cases, the obesity analyte signature comprises a presence of HTR2C, GNB3, FTO, isocaproic acid, beta-aminoisobutyric acid, butyric acid, allo-isoleucine, tryptophan and glutamine. In some cases, the obesity analyte signature comprises a presence of a single nucleotide polymorphism (SNP) or set of SNPs in or near HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, and/or BBS1.

In one embodiment, the obesity phenotype of the subject is identified to be abnormal satiation (hungry brain), and wherein the predetermined diet administered to the subject is a volumetrics diet comprising increased dietary fiber. In some cases, the increased dietary fiber is represented by a dietary fiber intake of greater than 3.5 g/kcal, wherein g is grams of food. In some cases, the increased dietary fiber is represented by a dietary fiber intake of greater than 4 g/kcal, wherein g is grams of food. In some cases, the increased dietary fiber is represented by a dietary fiber content above 20 to 45 g/10 MJ or 25 to 45 g/10 MJ, wherein g is grams of food. In some cases, the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food. In some cases, the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food. In some cases, the increased dietary fiber is as compared to a recommended dietary fiber intake for the subject's demographic group. In some cases, the subject is female, and the recommended dietary fiber intake is 21-25 grams of dietary fiber per day. In some cases, the subject is a male, and the recommended dietary fiber intake is 30-38 grams of dietary fiber per day. In some cases, the Caloric content of the volumetric diet comprising increased fiber content is set to 500 Calories less than the basal resting energy expenditure (REE) of the subject. In some cases, the REE of the subject is measured by indirect calorimetry. In some cases, the subject consumes 1-2 meals per day. In some cases, the subject is permitted a single serving of a fruit or vegetable in addition to each meal. In some cases, the intervention responsive obesity analyte signature comprises a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine and ghrelin. In some cases, the intervention responsive obesity analyte signature comprises a presence of HTR2C, GNB3, FTO, isocaproic acid, beta-aminoisobutyric acid, butyric acid, allo-isoleucine, tryptophan and glutamine. In some cases, the intervention responsive obesity analyte signature comprise a presence of a SNP in or near HTR2C, POMC, NPY, AGRP, MC4R, GNB3, SERT, and/or BDNF, and wherein the HADS questionnaire result does not indicate an anxiety sub scale. In some cases, the method further comprises administrating an exercise regimen to the subject. In some cases, the exercise regimen comprises a combination of resistance training and cardiovascular training for at least 150 minutes per week. In some cases, the method further comprises administering one or more pharmacological interventions based on the subject's obesity phenotype. In some cases, the one or more pharmacological interventions is phentermine-topiramate pharmacotherapy.

In one embodiment, the obesity phenotype of the subject is identified to be abnormal satiety (hungry gut), and wherein the predetermined diet administered to the subject is a diet comprising protein supplementation. In some cases, the Caloric content of the diet comprising protein supplementation is set to 500 Calories less than the basal resting energy expenditure (REE) of the subject. In some cases, the REE of the subject is measured by indirect calorimetry. In some cases, the subject consumes 3-5 meals per day. In some cases, the protein supplementation comprises a pre-meal protein shake, and the method further comprises consuming the pre-meal protein shake at least 30 minutes prior to the meal. In some cases, the protein supplementation comprises a pre-meal protein snack and the method further comprises consuming the pre-meal protein snack at least 60 minutes prior to the meal. In some cases, the diet comprising protein supplementation comprises greater than 0.8 g/kg of body weight (BW) per day of protein. In some cases, the diet comprising protein supplementation comprises greater than 20% of total energy per day derived from protein. In some cases, the diet comprising protein supplementation comprises greater than 46 grams per day of protein if the subject is female or 56 grams per day if the subject is male. In some cases, the intervention responsive obesity analyte signature comprises a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY. In some cases, the intervention responsive obesity analyte signature comprises a presence of HTR2C, GNB3, FTO, isocaproic acid, beta-aminoisobutyric acid, butyric acid, allo-isoleucine, tryptophan and glutamine. In some cases, the intervention responsive obesity analyte signature comprise a presence of a SNP in or near PYY, GLP-1, MC4R, GPBAR1, TCF7L2, ADAR2A, PCSK, and/or TMEM18. In some cases, the method further comprises administrating an exercise regimen to the subject. In some cases, the exercise regimen comprises a combination of resistance training and cardiovascular training for at least 150 minutes per week. In some cases, the method further comprises administering one or more pharmacological interventions based on the subject's obesity phenotype. In some cases, the one or more pharmacological interventions is a long-acting GLP-1 receptor agonist. In some cases, said GLP-1 receptor agonist comprises liraglutide or semaglutide.

In one embodiment, the obesity phenotype of the subject is identified to be abnormal hedonic/behavior (emotional hunger), and wherein the predetermined diet administered to the subject is a low-calorie diet comprising an absence of snacks between meals. In some cases, the Caloric content of the low-calorie diet comprising an absence of snacks between meals is set to 500 Calories less than the basal resting energy expenditure (REE) of the subject. In some cases, the REE of the subject is measured by indirect calorimetry. In some cases, the subject consumes 3 meals per day. In some cases, the intervention responsive obesity analyte signature comprises a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and wherein the HADS questionnaire result indicates a positive in an anxiety subscale. In some cases, the intervention responsive obesity analyte signature comprises a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin, and wherein the HADS questionnaire result is positive in an anxiety component. In some cases, the intervention responsive obesity analyte signature comprises a presence of HTR2C, GNB3, FTO, isocaproic acid, beta-aminoisobutyric acid, butyric acid, allo-isoleucine, tryptophan and glutamine. In some cases, the intervention responsive obesity analyte signature comprise a presence of a SNP in or near SLC6A4/SERT and/or DRD2 OR a SNP in TCF7L2, UCP3 and/or ADRA2A; wherein the HADS questionnaire result is positive in an anxiety component. In some cases, the method further comprises administrating an exercise regimen to the subject. In some cases, the exercise regimen comprises a combination of resistance training and cardiovascular training. In some cases, the method further comprises subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change.

In some cases, the method further comprises administering one or more pharmacological interventions based on the subject's obesity phenotype. In some cases, the one or more pharmacological interventions is naltrexone-bupropion pharmacotherapy.

In one embodiment, the obesity phenotype of the subject is identified to be slow metabolism (slow burn), and wherein the predetermined diet administered to the subject is a low-calorie diet comprising post-exercise protein shake or protein snack. In some cases, the Caloric content of the low-calorie diet comprising an absence of snacks between meals is set to 500 Calories less than the basal resting energy expenditure (REE) of the subject. In some cases, the REE of the subject is measured by indirect calorimetry. In some cases, the subject consumes 3 meals per day. In some cases, the method further comprises consuming the post-exercise protein shake or protein snack at least 30 minutes after ending an exercise session. In some cases, the intervention responsive obesity analyte signature comprises a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine, and wherein said HADS questionnaire result indicates a positive in an anxiety subscale. In some cases, the intervention responsive obesity analyte signature comprises a presence of HTR2C, GNB3, FTO, isocaproic acid, beta-aminoisobutyric acid, butyric acid, allo-isoleucine, tryptophan and glutamine. In some cases, the intervention responsive obesity analyte signature comprise a presence of a SNP in or near FTO, LEP, LEPR, UCP1, UCP2, UCP3, ADRA2, KLF14, NPC1, LYPLAL1, ADRB2, ADRB3 and/or BBS1; wherein the HADS questionnaire result indicates a positive in an anxiety sub scale. In some cases, the method further comprises administrating an exercise regimen to the subject. In some cases, the exercise regimen comprises a combination of resistance training and high intensity interval training (HIIT) training. In some cases, the method further comprises administering one or more pharmacological interventions based on the subject's obesity phenotype. In some cases, the one or more pharmacological interventions is phentermine pharmacotherapy.

In another aspect, provided herein is a method of treating obesity in a subject in need thereof, the method comprising administering a predetermined diet to a subject, wherein the subject has an abnormal satiation (hungry brain) obesity phenotype, and wherein the predetermined diet is a volumetrics diet comprising increased dietary fiber. In some cases, the increased dietary fiber is represented by a dietary fiber intake of greater than 3.5 g/kcal, wherein g is grams of food. In some cases, the increased dietary fiber is represented by a dietary fiber intake of greater than 4 g/kcal, wherein g is grams of food. In some cases, the increased dietary fiber is represented by a dietary fiber content above 20 to 45 g/10 MJ or 25 to 45 g/10 MJ, wherein g is grams of food. In some cases, the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food. In some cases, the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food. In some cases, the increased dietary fiber is as compared to a recommended dietary fiber intake for the subject's demographic group. In some cases, the subject is a female, and the recommended dietary fiber intake is 21-25 grams of dietary fiber per day. In some cases, the subject is a male, and the recommended dietary fiber intake is 30-38 grams of dietary fiber per day. In some cases, the Caloric content of the volumetric diet comprising increased fiber content is set to 500 Calories less than the basal resting energy expenditure (REE) of the subject. In some cases, the REE of the subject is measured by indirect calorimetry. In some cases, the subject consumes 1-2 meals per day. In some cases, the subject is permitted a single serving of a fruit or vegetable in addition to each meal. In some cases, the administration of the predetermined diet is effective to reduce the total body weight of the subject by at least 4%. In some cases, the administration of the predetermined diet is effective to reduce the total body weight of the subject by 2% to 8%. In some cases, the administration of the predetermined diet is effective to reduce the waist circumference of the subject by from about 1 inches to about 10 inches. In some cases, the method further comprises administrating an exercise regimen to the subject. In some cases, the exercise regimen comprises a combination of resistance training and cardiovascular training. In some cases, the method further comprises administering one or more pharmacological interventions based on the subject's obesity phenotype. In some cases, the one or more pharmacological interventions is phentermine-topiramate pharmacotherapy. In some cases, the abnormal satiation phenotype is characterized by the subject having a Hospital Anxiety and Depression Scale (HADS) questionnaire result that does not indicate an anxiety subscale in combination with an obesity analyte signature that comprises detection of a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine, ghrelin in a sample obtained from the subject OR a presence of HTR2C, GNB3, FTO, isocaproic acid, beta-aminoisobutyric acid, butyric acid, allo-isoleucine, tryptophan and glutamine AND a SNP in or near HTR2C, POMC, NPY, AGRP, MC4R, GNB3, SERT, and/or BDNF in a sample obtained from the subject. In some cases, the SNP is rs1414334. In some cases, the sample is selected from the group consisting of a blood sample, a saliva sample, a urine sample, a breath sample, and a stool sample.

In another aspect, provided herein is a method of treating obesity in a subject in need thereof, the method comprising administering a predetermined diet to a subject, wherein the subject has an abnormal satiety (hungry gut) obesity phenotype, and wherein the predetermined diet is a diet comprising protein supplementation. In some cases, the Caloric content of the diet comprising protein supplementation is set to 500 Calories less than the basal resting energy expenditure (REE) of the subject. In some cases, the REE of the subject is measured by indirect calorimetry. In some cases, the subject consumes 3-5 meals per day.

In some cases, the protein supplementation comprises a pre-meal protein shake, and the method further comprises consuming the pre-meal protein shake at least 30 minutes prior to the meal. In some cases, the protein supplementation comprises a pre-meal protein snack and the method further comprises consuming the pre-meal protein snack at least 60 minutes prior to the meal. In some cases, the diet comprising protein supplementation comprises greater than 0.8 g/kg of body weight (BW) per day of protein. In some cases, the diet comprising protein supplementation comprises greater than 20% of total energy per day derived from protein. In some cases, the diet comprising protein supplementation comprises greater than 46 grams per day of protein if the subject is female or 56 grams per day if the subject is male. In some cases, the administration of the predetermined diet is effective to reduce the total body weight of the subject by at least 4%. In some cases, the administration of the predetermined diet is effective to reduce the total body weight of the subject by 2% to 8%. In some cases, the administration of the predetermined diet is effective to reduce the waist circumference of the subject by from about 1 inches to about 10 inches. In some cases, the method further comprises administrating an exercise regimen to the subject. In some cases, the exercise regimen comprises a combination of resistance training and cardiovascular training. In some cases, the method further comprises administering one or more pharmacological interventions based on the subject's obesity phenotype. In some cases, the one or more pharmacological interventions is a GLP-1 receptor agonist. In some cases, said GLP-1 receptor agonist comprises liraglutide. In some cases, the abnormal satiety phenotype is characterized by the subject having a Hospital Anxiety and Depression Scale (HADS) questionnaire result that does not indicate an anxiety subscale in combination with an obesity analyte signature that comprises detection of a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY in a sample obtained from the subject OR a presence of HTR2C, GNB3, FTO, isocaproic acid, beta-aminoisobutyric acid, butyric acid, allo-isoleucine, tryptophan and glutamine AND a SNP in or near PYY, GLP-1, MC4R, GPBAR1, TCF7L2, ADAR2A, PCSK, and/or TMEM18 in a sample obtained from the subject. In some cases, the SNP is rs7903146. In some cases, the sample is selected from the group consisting of a blood sample, a saliva sample, a urine sample, a breath sample, and a stool sample.

In another aspect, provided herein is a method of treating obesity in a subject in need thereof, the method comprising administering a predetermined diet to a subject, wherein the subject has an abnormal hedonic/behavior (emotional hunger), and wherein the predetermined diet administered to the subject is a low-calorie diet comprising an absence of snacks between meals. In some cases, the Caloric content of the high protein diet comprising pre-meal protein shake or protein snack is set to 500 Calories less than the basal resting energy expenditure (REE) of the subject. In some cases, the REE of the subject is measured by indirect calorimetry. In some cases, the subject consumes 3 meals per day. In some cases, the administration of the predetermined diet is effective to reduce the total body weight of the subject by at least 4%. In some cases, the administration of the predetermined diet is effective to reduce the total body weight of the subject by 2% to 8%. In some cases, the administration of the predetermined diet is effective to reduce the waist circumference of the subject by from about 1 inches to about 10 inches. In some cases, the method further comprises administrating an exercise regimen to the subject. In some cases, the exercise regimen comprises a combination of resistance training and cardiovascular training. In some cases, the method further comprises subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change. In some cases, the method further comprises administering one or more pharmacological interventions based on the subject's obesity phenotype. In some cases, the one or more pharmacological interventions is a naltrexone-bupropion pharmacotherapy. In some cases, the abnormal hedonic/behavior phenotype is characterized by the subject having a Hospital Anxiety and Depression Scale (HADS) questionnaire result that indicates an anxiety subscale in combination with an obesity analyte signature that comprises detection of a presence of a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY in a sample obtained from the subject; a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin in a sample obtained from the subject; OR a presence of HTR2C, GNB3, FTO, isocaproic acid, beta-aminoisobutyric acid, butyric acid, allo-isoleucine, tryptophan and glutamine AND a presence of a SNP in or near SLC6A4/SERT and/or DRD2; OR a presence of HTR2C, GNB3, FTO, isocaproic acid, beta-aminoisobutyric acid, butyric acid, allo-isoleucine, tryptophan and glutamine AND a SNP in or near TCF7L2, UCP3 and/or ADRA2A in a sample obtained from the subject. In some cases, the SNP is rs4795541 or rs1626521. In some cases, the sample is selected from the group consisting of a blood sample, a saliva sample, a urine sample, a breath sample, and a stool sample.

In another aspect, provided herein is a method of treating obesity in a subject in need thereof, the method comprising administering a predetermined diet to a subject, wherein the subject has a slow metabolism (slow burn) obesity phenotype, and wherein the predetermined diet administered to the subject is a low-calorie diet comprising post-exercise protein shake or protein snack. In some cases, the Caloric content of the high protein diet comprising pre-meal protein shake or protein snack is set to 500 Calories less than the basal resting energy expenditure (REE) of the subject. In some cases, the REE of the subject is measured by indirect calorimetry. In some cases, the subject consumes 3 meals per day. In some cases, the method further comprises consuming the post-exercise protein shake or protein snack at least 30 minutes after ending an exercise session. In some cases, the administration of the predetermined diet is effective to reduce the total body weight of the subject by at least 4%. In some cases, the administration of the predetermined diet is effective to reduce the total body weight of the subject by 2% to 8%. In some cases, the administration of the predetermined diet is effective to reduce the waist circumference of the subject by from about 1 inches to about 10 inches. In some cases, the method further comprises administrating an exercise regimen to the subject. In some cases, the exercise regimen comprises a combination of resistance training and high intensity interval training (HIIT) training. In some cases, the method further comprises administering one or more pharmacological interventions based on the subject's obesity phenotype. In some cases, the one or more pharmacological interventions is a phentermine pharmacotherapy. In some cases, the abnormal satiation phenotype is characterized by the subject having a Hospital Anxiety and Depression Scale (HADS) questionnaire result that indicates an anxiety subscale in combination with an obesity analyte signature that comprises detection of a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine in a sample obtained from the subject OR a presence of HTR2C, GNB3, FTO, isocaproic acid, beta-aminoisobutyric acid, butyric acid, allo-isoleucine, tryptophan and glutamine AND a SNP in or near FTO, LEP, LEPR, UCP1, UCP2, UCP3, ADRA2, KLF14, NPC1, LYPLAL1, ADRB2, ADRB3 and/or BBS1 in a sample obtained from the subject. In some cases, the SNP is rs2075577. In some cases, the sample is selected from the group consisting of a blood sample, a saliva sample, a urine sample, a breath sample, and a stool sample.

### BRIEF DESCRIPTION OF THE FIGURES

FIG. 1 depicts the advised caloric intake, macronutrient composition and timing of the phenotype-guided diets for each of the four (4) pathophysiology obesity classifications described herein (i.e., Hungry Brain, Hungry Gut, Emotional Hunger and Slow Burn). Important to note: Calorie level followed by the patient is the resting energy expenditure (REE) −500 calories, for patients whose REE is quite low, calorie level will not go below 900 calories.

FIG. 2 depicts the advised lifestyle intervention for each of the four (4) pathophysiology obesity classifications described herein (i.e., Hungry Brain, Hungry Gut, Emotional Hunger and Slow Burn).

FIG. 3A illustrates that the mean weight change from baseline was −7.1% (4.6%) with PLI vs −3.5% (4.2%) with SLI (referred to as MCD) (difference, −3.6 percentage points [95% CI, −5.1 to −2.2]; P<0.001) at week 12. FIG. 3B illustrates the mean weight change from baseline for the PLI (Pheno) group vs the SLI (MCD) group for each of the obesity phenotypes.

FIG. 4 illustrate the percentage of participants in either the standard lifestyle intervention (SLI) or phenotype-guided lifestyle intervention (PLI) groups that have lost at least 5% or 10% of baseline body weight at 12 weeks.

FIGS. 5A-5F illustrates the difference or delta at 12 weeks from baseline for total daily caloric intake (FIG. 5A), calories to fullness (CTF) in the ad libitum meal (FIG. 5B), resting energy expenditure (REE) (FIG. 5C), lean mass percentage (FIG. 5D), gastric emptying (FIG. 5E) and anxiety component of the HADS questionnaire (FIG. 5F) in the PLI or SLI groups.

FIG. 6 depicts a schematic of the iterative PLI-based system for prescribing lifestyle interventions described in Example 2.

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e. can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

The details of one or more embodiments of the invention are set forth in the accompanying drawings and the description below. Other features, objects, and advantages of the invention will be apparent from the description and drawings, and from the claims.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all be referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

As used herein, the term “Calorie” or “kcal” can be used interchangeably and can generally refer to 1 Calorie (with a capital “C”) equaling 1 kcal, or 1000 calories (lower case “c”).

A low-fat diet is a diet that comprises about 3%, 5%, 10%, 15%, 20%, 25%, 30%, 40%, 50%, 60%, 70% or 80% less fat than the normal recommended amount of fat in a diet for a given individual of a given species of a given age, weight, and general health condition. For example, a low-fat diet in humans can comprise a diet consisting of about 0%, 3%, 5%, 7%, 10%, 13%, 15%, 20% or 25% fat.

A low carbohydrate diet is a diet that comprises about 3%, 5%, 10%, 15%, 20%, 25%, 30%, 40%, 50%, 60%, 70% or 80% less carbohydrates than the normal recommended amount of calories for a given individual of a given species of a given age, weight, and general health condition.

A low-calorie diet as used herein can be a diet that comprises about or exactly 3%, 5%, 10%, 15%, 20%, 25%, 30%, 40%, 50%, 60%, 70% or 80% less calories than the normal recommended amount of calories for a given individual of a given species of a given age, weight, and general health condition.

In some cases, a low-calorie diet as used herein can be a diet that provides to a subject a pre-determined number of Calories or kcals below a resting energy expenditure (REE) of said subject. Said low-calorie diet can comprise a specific percentage (e.g., about or exactly 3%, 5%, 10%, 15%, 20%, 25%, 30%, 40%, 50%, 60%, 70% or 80%) less calories than the normal recommended amount of calories for a given individual of a given species of a given age, weight, and general health condition. In one embodiment, a low-calorie diet (LCD) as used herein can refer to creating or following a diet whereby a 500-kcal deficit from a subject's measured or calculated basal metabolic rate is created per day when said subject follows the prescribed diet. As used herein, an LCD can provide, for example, from about 800 to 1200 kcal per day. As used herein a “very low-calorie diet (VLCD)” can provide, for example, about 800 or fewer kcal per day.

The term “dietary fiber” as used herein can refer to the non-digestible form of carbohydrate found in plant foods, including fruits and vegetables, whole grain products, beans, nuts and seeds. It can provide bulk in the diet, which can be used to help promote healthy gastrointestinal function and contribute to a feeling of fullness after eating. Dietary fiber as used herein can comprise soluble dietary fiber and insoluble dietary fiber. The percentage or proportion of dietary fiber that is either soluble (i.e., water soluble) or insoluble (i.e., water insoluble) can be variable and can be dependent on the food source. Soluble dietary fiber can refer to dietary fiber that dissolves in water to form a thick gel-like substance in the stomach. Soluble dietary fiber can be broken down by bacteria in the large intestine and provide some calories. Insoluble dietary fiber can refer to dietary fiber that does not dissolve in water and may pass through the gastrointestinal tract relatively intact and, therefore, is not a source of calories.

A high-fiber diet as used herein can be a diet that comprises about or exactly 3%, 5%, 10%, 15%, 20%, 25%, 30%, 40%, 50%, 60%, 70% or 80% more dietary than the normal recommended amount of dietary fiber for a given individual of a given species of a given age, weight, and general health condition.

The term “volumetrics diet” as used herein can refer to a diet based on the premise that the volume of food eaten is critical, rather than the number of calories consumed. A volumetrics diet as provided herein can emphasize eating low-energy-dense, high-nutrient-dense foods like fruits, vegetables, whole grains and low-fat dairy. The volumetrics diet can be based on the idea that the volume of food you eat, rather the counting calories, leads to weight loss. When more food is consumed, a subject can experience a fullness in a psychological sense. The diet can follow more of an unstructured eating approach than one that restricts specific foods and severely limits a subject's daily caloric intake.

The term “weight loss” as used herein can refer to a reduction of the total body mass, due to a mean loss of fluid, body fat or adipose tissue and/or lean mass, namely bone mineral deposits, muscle, tendon, and other connective tissue.

The terms “ad libitum diet” as used herein can refer to a diet where the amount of daily calories intake of a subject is not restricted to a particular value. A subject following an ad libitum diet is free to eat until satiety.

The term “energy density” as used herein can refer to the amount of energy, as represented by the number of calories, in a specific weight of food.

The term “nutrient density” as used herein can refer to the balance of beneficial nutrients in a food (e.g., vitamins, minerals, lean protein, healthy fats and fiber) compared with nutrients to limit (e.g., saturated fat, sodium, added sugars and refined carbohydrates). Nutrient density can also refer to the amount of beneficial nutrients in a food product in proportion to e.g., energy content, weight or amount of detrimental nutrients. The terms such as nutrient rich and micronutrient dense can also refer to similar properties.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control. In addition, the materials, methods, and examples are illustrative only and not intended to be limiting.

### Overview

Provided herein are methods for predicting dietary weight loss success of a subject, for classifying responsiveness of a subject to a predetermined diet or lifestyle intervention, for selecting a successful weight loss diet for a subject and for maintaining weight or preventing weight re-gain of a subject. Some other aspects of the present disclosure also relate to methods for inducing weight loss and for treating overweight or obesity in a subject in need thereof. The methods of the present disclosure are all based on administering a predetermined diet or lifestyle intervention to a subject, based on the subject's obesity phenotype. In this way, the predetermined diet or lifestyle intervention can be personalized or individualized to any one or specific subject based on said subject's obesity phenotype. As provided herein, a lifestyle intervention can refer to an intervention that comprises dietary guidance, guidance on physical activity or exercise regimen and/or behavioral intervention. The dietary guidance can entail a predetermined diet. Guidance on physical activity can entail preparing, designing or outlining an exercise regimen. Behavioral intervention can entail a plan or regimen for behavioral counseling or behavior modification. In some cases, the lifestyle intervention is aligned to or determined by the subject's obesity phenotype and can be referred to as a phenotype guided or tailored lifestyle intervention or PLI. In some cases, the methods provided entail assessing or determining a subject's obesity phenotype and then administering a dietary intervention alone or in combination with one or more additional interventions (e.g., pharmacological intervention, surgical intervention, weight loss device, exercise or physical activity intervention, behavior intervention, and/or microbiome intervention) used to treat obesity. In some cases, the methods provided entail assessing or determining a subject's obesity phenotype and then administering a phenotype-guided lifestyle intervention (PLI) alone or in combination with one or more additional interventions (e.g., pharmacological intervention, surgical intervention, weight loss device and/or microbiome intervention) used to treat obesity.

A subject can be a mammal. Any type of mammal can be assessed and/or treated as described herein. Examples of mammals that can be assessed and/or treated using the methods as described herein include, without limitation, primates (e.g., humans and monkeys), dogs, cats, horses, cows, pigs, sheep, rabbits, mice, and rats. In some cases, the mammal can be a human. In some cases, a mammal can be an obese mammal or a mammal with or suffering from obesity. For example, humans with obesity can be assessed for intervention (e.g., lifestyle intervention and/or a pharmacological intervention) responsiveness and treated with one or more interventions as described herein. In cases where the mammal is a human, the human can be of any race. For example, a human can be Caucasian or Asian.

Any appropriate method can be used to identify a mammal as being overweight (e.g., as being obese). In some cases, calculating body mass index (BMI), measuring waist and/or hip circumference, health history (e.g., weight history, weight-loss efforts, exercise habits, eating patterns, other medical conditions, medications, stress levels, and/or family health history), physical examination (e.g., measuring your height, checking vital signs such as heart rate, blood pressure, listening to your heart and lungs, and examining your abdomen), percentage of body fat and distribution, percentage of visceral and organs fat, metabolic syndrome, and/or obesity related comorbidities can be used to identify mammals (e.g., humans) as being obese. For example, a BMI of greater than about 30 kg/m² can be used to identify mammals (e.g., Caucasian humans) as being obese. For example, a BMI of greater than about 27 kg/m² with a co-morbidity can be used to identify mammals (e.g., Asian humans) as being obese.

When treating obesity in a mammal (e.g., a human) as described herein, the mammal can also have one or more obesity-related (e.g., weight-related) co-morbidities. Examples of weight-related co-morbidities include, without limitation, hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis, depression, anxiety, and atherosclerosis (coronary artery disease and/or cerebrovascular disease). In some cases, the methods and materials described herein can be used to treat one or more obesity-related co-morbidities.

Once identified or diagnosed as being obese, a mammal can be assessed to determine whether or not said mammal is likely to respond to one or more interventions (e.g., pharmacological intervention, surgical intervention, weight loss device, lifestyle intervention, diet intervention, behavior intervention, and/or microbiome intervention). For example, a sample obtained from the mammal can be assessed for diet intervention, lifestyle intervention and/or pharmacological intervention responsiveness. As described herein, measurement or analysis of a panel of obesity analytes in a sample obtained from an obese mammal can be used to determine an obesity analyte signature of the mammal and can be used in to determine an obesity phenotype of the mammal.

A sample can be any type of sample that can be obtained from a subject. In some cases, a sample can be a biological sample. In some cases, a sample can contain obesity analytes (e.g., DNA, RNA, proteins, peptides, metabolites, hormones, and/or exogenous compounds (e.g., medications)). Examples of samples that can be used in the methods as described herein include, without limitation, fluid samples (e.g., blood, serum, plasma, urine, saliva, sweat, or tears), breath samples, cellular samples (e.g., buccal samples), tissue samples (e.g., adipose samples), stool samples, gastro samples, and intestinal mucosa samples. In some cases, a sample (e.g., a blood sample) can be collected while the mammal is fasting (e.g., a fasting sample such as a fasting blood sample). In some cases, a sample can be processed (e.g., to extract and/or isolate obesity analytes). For example, a serum sample can be obtained from a mammal with obesity and can be assessed to determine if the obese mammal is likely to be responsive to one or more interventions (e.g., pharmacological intervention, surgical intervention, weight loss device, diet intervention, lifestyle intervention, behavior intervention, and/or microbiome intervention) based, at least in part, on an obesity phenotype, which is based, at least in part, on determining an obesity analyte signature in the sample from the obese mammal. For example, a urine sample can be obtained from an obese mammal and can be analyzed (e.g., have a plurality of obesity-related analytes measured) to determine if the obese mammal is likely to be responsive to any intervention described herein (e.g., diet and/or lifestyle intervention) based, at least in part, on an obesity phenotype, which is based, at least in part, on an obesity analyte signature representative of the plurality of obesity-related analytes analyzed in the sample.

When treating obesity in a subject as described herein, the intervention (e.g., dietary intervention or lifestyle intervention) can be effective to reduce the weight, reduce the waist circumference and/or slow or prevent weight gain of the subject. For example, any treatment described herein can be effective to reduce the weight (e.g., the total body weight) of an obese subject by at least 1%, 2%, 3%, 4%, 5%, 6%, 7%, 8%, 9%,10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%, as compared to baseline. Treatment described herein can be effective to reduce the weight (e.g., the total body weight) of an obese subject by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%, as compared to baseline. Treatment described herein can be effective to reduce the weight (e.g., the total body weight) of an obese subject by at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%, as compared to baseline. In some cases, the intervention described herein (e.g., dietary intervention or lifestyle intervention) can be effective to reduce the weight (e.g., the total body weight) of an obese subject by at least 3%, at least 5%, at least 8%, at least 10%, at least 12%, at least 15%, at least 18%, at least 20%, at least 22%, at least 25%, at least 28%, at least 30%, at least 33%, at least 36%, at least 39%, or at least 40%), as compared to baseline. For example, the intervention described herein (e.g., dietary intervention or lifestyle intervention) can be effective to reduce the weight (e.g., the total body weight) of a subject by from about 3% to about 40% (e.g., from about 3% to about 35%, from about 3% to about 30%, from about 3% to about 25%, from about 3% to about 20%, from about 3% to about 15%, from about 3% to about 10%, from about 3% to about 5%, from about 5% to about 40%, from about 10% to about 40%, from about 15% to about 40%, from about 20% to about 40%, from about 25% to about 40%, from about 35% to about 40%, from about 5% to about 35%, from about 10% to about 30%, from about 15% to about 25%, or from about 18% to about 22%), as compared to baseline. For example, the intervention described herein (e.g., dietary intervention or lifestyle intervention) can be effective to reduce the weight (e.g., the total body weight) of a subject by from about 3 kg to about 100 kg (e.g., about 5 kg to about 100 kg, about 8 kg to about 100 kg, about 10 kg to about 100 kg, about 15 kg to about 100 kg, about 20 kg to about 100 kg, about 30 kg to about 100 kg, about 40 kg to about 100 kg, about 50 kg to about 100 kg, about 60 kg to about 100 kg, about 70 kg to about 100 kg, about 80 kg to about 100 kg, about 90 kg to about 100 kg, about 3 kg to about 90 kg, about 3 kg to about 80 kg, about 3 kg to about 70 kg, about 3 kg to about 60 kg, about 3 kg to about 50 kg, about 3 kg to about 40 kg, about 3 kg to about 30 kg, about 3 kg to about 20 kg, about 3 kg to about 10 kg, about 5 kg to about 90 kg, about 10 kg to about 75 kg, about 15 kg to about 50 kg, about 20 kg to about 40 kg, or about 25 kg to about 30 kg), as compared to baseline. For example, the intervention described herein (e.g., dietary intervention or lifestyle intervention) can be effective to reduce the waist circumference of a subject by from about 1 inches to about 10 inches (e.g., about 1 inches to about 9 inches, about 1 inches to about 8 inches, about 1 inches to about 7 inches, about 1 inches to about 6 inches, about 1 inches to about 5 inches, about 1 inches to about 4 inches, about 1 inches to about 3 inches, about 1 inches to about 2 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 9 inches to about 10 inches, about 2 inches to about 9 inches, about 3 inches to about 8 inches, about 4 inches to about 7 inches, or about 5 inches to about 7 inches), as compared to baseline. The baseline can be weight (e.g., the total body weight) and/or the waist circumference of the subject prior to administration of the intervention described herein (e.g., dietary intervention or lifestyle intervention).

### Obesity Phenotyping

Any appropriate method can be used to identify an obesity phenotype of a subject as described herein. For example, an obesity phenotype of a subject can be ascertained using any of the methods described in US20210072259A1, which is herein incorporated by reference in its entirety. In some cases, the obesity phenotype of a subject suffering from obesity can comprise genetic and/or non-genetic multi-omic data obtained from the subject or a sample obtained from the subject suffering from obesity. In some cases, the obesity phenotype of a subject can be identified by determining the metabolome, the genome, the proteome, and/or the peptidome of the subject (e.g., an obese mammal). In some cases, the obesity phenotype of the subject can be identified by determining a profile (e.g., expression profile) of a series of analytes associated with obesity (e.g., obesity analytes), which can be referred to as the obesity analyte signature of the subject, in a sample (e.g., in a sample obtained from the subject). In some cases, the obesity analyte signature can comprise genetic and/or non-genetic multi-omic data obtained from an individual or a sample obtained from an individual suffering from obesity. In some cases, the obesity analyte signature can be obtained by detecting the presence, absence, or level of one or more metabolites, detecting the presence, or absence, or level one or more peptides (e.g., gastrointestinal peptides), and/or detecting the presence or absence of one or more single nucleotide polymorphisms (SNPs) in the sample obtained from the subject (e.g., the obese mammal). The multi-omic data can be selected from the group consisting of metabolomic data, genomic data, proteomic data, peptidomic data and any combination thereof. In some cases, the multi-omic data obtained for an individual or a sample obtained from an individual represents an obesity analyte signature for that individual.

An obesity analyte signature can include any appropriate analyte. Examples of analytes that can be included in an obesity analyte signature described herein can include, without limitation, DNA, RNA, proteins, peptides, metabolites, hormones, and exogenous compounds (e.g., medications). In some cases, the obesity analyte signature can be obtained by detecting the presence, absence, or level of one or more metabolites, detecting the presence, or absence, or level one or more peptides (e.g., gastrointestinal peptides), and/or detecting the presence or absence of one or more single nucleotide polymorphisms (SNPs). An obesity analyte signature can be evaluated using any appropriate methods. For example, metabolomics, genomics, microbiome analysis, proteomic, peptidomics, and behavioral questionnaires can be used to evaluate and/or identify an obesity analyte signature described herein.

A metabolite can be any metabolite that is associated with obesity. In some cases, a metabolite can be an amino-compound. In some cases, a metabolite can be a neurotransmitter. In some cases, a metabolite can be a fatty acid (e.g., a short chain fatty acid). In some cases, a metabolite can be an amino compound. In some cases, a metabolite can be a bile acid. Examples of metabolites that can be used to determine the obesity analyte signature of a subject in a sample (e.g., in a sample obtained from an obese mammal) can include, without limitation, 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, phenylalanine gamma-aminobutyric acid, acetic acid, histidine, LCA, ghrelin, ADRA2A, cholesterol, glucose, acetylcholine, propionic acid, CDCA, PYY, ADRA2C, insulin, adenosine, isobutyric acid, 1-methylhistidine, DCA, CCK, GNB3, glucagon, aspartate, butyric acid, 3-methylhistidine, UDCA, GLP-1, FTO, leptin, dopamine, valeric acid, asparagine, HDCA, GLP-2, MC4R, adiponectin, D-serine, isovaleric acid, phosphoethanolamine, CA, glucagon, TCF7L2, glutamate, hexanoic acid, arginine, GLCA, oxyntomodulin, 5-HTTLPR, glycine, octanoic acid, carnosine, GCDCA, neurotensin, HTR2C, myristic acid, taurine, GDCA, FGF, UCP2, norepinephrine, palmitic acid, anserine, GUDCA, GIP, UCP3, serotonin, palmitoleic acid, serine, GHDCA, OXM, GPBAR1, taurine, palmitelaidic acid, glutamine, GCA, FGF19, NR1H4, stearic acid, ethanolamine, TLCA, FGF21, FGFR4, oleic acid, glycine, TCDCA, LDL, elaidic acid, aspartic acid, TDCA, insulin, GLP-1, linoleic acid, sarcosine, TUDCA, glucagon, CCK, a-linolenic, proline, THDCA, amylin, arachidonic acid, alpha-aminoadipic-acid, TCA, pancreatic polypeptide, eicosapentaenoic acid, DHCA, neurotensin, docosahexaenoic acid, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, and phenylalanine.

A gastrointestinal peptide can be any gastrointestinal peptide that is associated with obesity. In some cases, a gastrointestinal peptide can be a peptide hormone. In some cases, a gastrointestinal peptide can be released from gastrointestinal cells in response to feeding. Examples of gastrointestinal peptides that can be used to determine the obesity analyte signature in a sample (e.g., in a sample obtained from an obese mammal) can include, without limitation, ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, and pancreatic polypeptide.

A SNP can be any SNP that is associated with obesity. A SNP can be in a coding sequence (e.g., in a gene) or a non-coding sequence. For example, in cases where a SNP is in a coding sequence, the coding sequence can be any appropriate coding sequence. Examples of coding sequences that a SNP associated with obesity can be in or near include, without limitation, ADRA2A, ADRA2C, GNB3, FTO, MC4R, TCF7L2, 5-HTTLPR, HTR2C, UCP2, UCP3, GPBAR1, NR1H4, FGFR4, PYY, GLP-1, CCK, leptin, adiponectin, neurotensin, ghrelin, GLP-1 receptor, GOAT, DPP4, POMC, NPY, AGRP, SERT, BDNF, SLC6A4, DRD2, LEP, LEPR, UCP1, KLF14, NPC1, LYPLAL1, ADRB2, ADRB3, BBS1, ACSL6, ADARB2, ADCY8, ADH1B, AJAP1, ATP2C2, ATP6VOD2, C21orf7, CAMKMT, CAP2, CASC4, CD48, CDC42SE2, CDYL, CES5AP1, CLMN, CNPY4, COL19A1, COL27A1, COL4A3, COROIC, CPZ, CTIF, DAAM2, DCHS2, DOCKS, EGFLAM, FAM125B, FAM71E2, FRMD3, GALNTL4, GLT1D1, HHAT, KRT23, LHPP, LINC00578, LINC00620, LIPC, LOC100128714, LOC100287160, LOC100289473, LOC100293612|LINC00620, LOC100506869, LOC100507053, LOC100507053|ADH1A, LOC100507053|ADH, LOC100507443, LOC100996571|CYYR1, LOC152225, LOC255130, LPAR1, LUZP2, MCM7, MICAL3, MMS19, MYBPC1, NR2F2-AS1, NSMCE2, NTN1, 03FAR1, OAZ2, OSBP2, P4HA2, PADI1, PARD3B, PARK2, PCDH15, PIEZO2, PKIB, PRH1-PRR4, PTPRD, RALGPS1|ANGPTL2, RPS24P10, RTN4RL1, RYR2, SCN2A, SEMA3C, SEMASA, SFMBT2, SGCG, SLC22A15, SLC2A2, SLCO1B1, SMOC2,SNCAIP, SNX18, SRRM4, SUSD1, TBC1D16, TCERG1L, TENM3, TJP3, TLL1, TMEM9B, TPM1, VTI1A, VWF, WWOX, WWTR1, ZFYVE28, ZNF3, ZNF609, and ZSCAN21. Examples of SNPS that can be used to determine the obesity analyte signature in a sample (e.g., in a sample obtained from an obese mammal) can include, without limitation, rs657452, rs11583200, rs2820292, rs11126666, rs11688816, rs1528435, rs7599312, rs6804842, rs2365389, rs3849570, rs16851483, rs17001654, rs11727676, rs2033529, rs9400239, rs13191362, rs1167827, rs2245368, rs2033732, rs4740619, rs6477694, rs1928295, rs10733682, rs7899106, rs17094222, rs11191560, rs7903146, rs2176598, rs12286929, rs11057405, rs10132280, rs12885454, rs3736485, rs758747, rs2650492, rs9925964, rs1000940, rs1808579, rs7243357, rs17724992, rs977747, rs1460676, rs17203016, rs13201877, rs1441264, rs7164727, rs2080454, rs9914578, rs2836754, rs492400, rs16907751, rs9374842, rs9641123, rs9540493, rs4787491, rs6465468, rs7239883, rs3101336, rs12566985, rs12401738, rs11165643, rs17024393, rs543874, rs13021737, rs10182181, rs1016287, rs2121279, rs13078960, rs1516725, rs10938397, rs13107325, rs2112347, rs205262, rs2207139, rs17405819, rs10968576, rs4256980, rs11030104, rs3817334, rs7138803, rs12016871, rs12429545, rs11847697, rs7141420, rs16951275, rs12446632, rs3888190, rs1558902, rs12940622, rs6567160, rs29941, rs2075650, rs2287019, rs3810291, rs7715256, rs2176040, rs6091540, rs1800544, Ins-Del-322, rs5443, rs1129649, rs1047776, rs9939609, rs17782313, rs7903146, rs4795541, rs3813929, rs518147, rs1414334, rs659366, -3474, rs2075577, rs15763, rs1626521, rs11554825, rs4764980, rs434434, rs351855, and rs2234888.

An obesity analyte signature described herein can include any appropriate combination of analytes. For example, an obesity analyte signature can include 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY. For example, when an obesity analyte signature includes 9 analytes, the analytes can include HTR2C, GNB3, FTO, isocaproic acid, beta-aminoisobutyric-acid, butyric acid, allo-isoleucine, tryptophan, and glutamine.

Any appropriate method can be used to detect the presence, absence, or level of an obesity analyte within a sample. For example, mass spectrometry (e.g., triple-stage quadrupole mass spectrometry coupled with ultra-performance liquid chromatography (UPLC)), radioimmunoassays, enzyme-linked immunosorbent assays, sequencing techniques (e.g., PCR-based sequencing techniques), and/or restriction fragment length polymorphism (RFLP) can be used to determine the presence, absence, or level of one or more analytes associated with obesity in a sample.

In some cases, identifying the obesity phenotype can include obtaining results from one or more questionnaires. A questionnaire can be associated with obesity. A questionnaire can be associated with anxiety. In some cases, a questionnaire can be answered the time of the assessment. In some cases, a questionnaire can be answered prior to the time of assessment.

In some cases, a questionnaire can be answered following administration of one or more interventions (e.g., pharmacological intervention, surgical intervention, weight loss device, diet intervention, lifestyle intervention, behavior intervention, and/or microbiome intervention) as provided herein. For example, when a questionnaire is answered prior to the time of the assessment, the questionnaire results can be obtained by reviewing a patient history (e.g., a medical chart). A questionnaire can be a behavioral questionnaire (e.g., psychological welfare questionnaires, alcohol use questionnaires, eating behavior questionnaires, body image questionnaires, physical activity level questionnaire, and weight management questionnaires. Examples of questionnaires that can be used to determine the obesity phenotype of a mammal (e.g., an obese mammal) include, without limitation, The Hospital Anxiety and Depression Scale (HADS) questionnaire, The Hospital Anxiety and Depression Inventory questionnaire, The Questionnaire on Eating and Weight Patterns, The Weight Efficacy Life-Style (WEL) Questionnaire, Three-Factor Eating Questionnaire (TFEQ), and The Multidimensional Body-Self Relations Questionnaire. For example, a questionnaire can be a HADS questionnaire.

In some cases, an obesity analyte signature can include the presence of serotonin, glutamine, isocaproic acid, allo-isoleucine, hydroxyproline, beta-aminoisobutyric-acid, alanine, hexanoic acid, tyrosine, and PYY. For example, an obesity phenotype Group 1 (hungry brain or abnormal satiation) can have an obesity analyte signature that includes the presence of serotonin, glutamine, isocaproic acid, allo-isoleucine, hydroxyproline, beta-aminoisobutyric-acid, alanine, hexanoic acid, tyrosine, and PYY. For example, an obesity phenotype Group 1 (hungry brain) can have an obesity analyte signature that has an absence of (e.g., lacks the presence of) 1-methylhistine, gamma-amino-n-butyric-acid, phenylalanine, ghrelin, and includes a HADS questionnaire result that does not indicate an anxiety subscale (HADS-A; e.g., includes a HADS-A questionnaire result).

In some cases, an obesity analyte signature can include the presence of 1-methylhistine, allo-isoleucine, hydroxyproline, beta-aminoisobutyric-acid, alanine, and phenylalanine. For example, an obesity phenotype Group 2 (hungry gut or abnormal satiety) can have an obesity analyte signature that includes the presence of 1-methylhistine, allo-isoleucine, hydroxyproline, beta-aminoisobutyric-acid, alanine, and phenylalanine. For example, an obesity phenotype Group 2 (hungry gut) can have an obesity analyte signature that has an absence of (e.g., lacks the presence of) serotonin, glutamine, gamma-amino-n-butyric-acid, isocaproic acid, hexanoic acid, tyrosine, ghrelin, PYY, and does not include a HADS questionnaire result that indicates an anxiety subscale (e.g., does not include a HADS-A questionnaire result).

In some cases, an obesity analyte signature can include the presence of serotonin and can include a HADS-A questionnaire. For example, an obesity phenotype Group 3 (emotional or hedonic eating) can have an obesity analyte signature that includes serotonin and includes a HADS-A questionnaire result that indicates anxiety on an anxiety subscale. For example, an obesity phenotype Group 3 (emotional or hedonic eating or behavioral eating) can have an obesity analyte signature that has an absence of (e.g., lacks the presence of) 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY.

In some cases, an obesity analyte signature can include the presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and includes a HADS-A questionnaire result that indicates anxiety on an anxiety subscale. For example, an obesity phenotype Group 4 (emotional or hedonic eating or behavioral eating) can have an obesity analyte signature that includes 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and includes a HADS-A questionnaire result that indicates anxiety on an anxiety subscale. For example, an obesity phenotype Group 4 (emotional or hedonic eating) can have an obesity analyte signature that has an absence of (e.g., lacks the presence of) serotonin, hydroxyproline, and ghrelin.

In some cases, an obesity analyte signature can include the presence of serotonin, beta-aminoisobutyric-acid, alanine, hexanoic acid, phenylalanine, and includes a HADS-A questionnaire result that indicates anxiety on an anxiety subscale. For example, an obesity phenotype Group 5 (mixed obesity phenotype) can have an obesity analyte signature that includes the presence of serotonin, beta-aminoisobutyric-acid, alanine, hexanoic acid, phenylalanine, and includes a HADS-A questionnaire result that indicates anxiety on an anxiety subscale. For example, an obesity phenotype Group 5 (mixed obesity phenotype) can have an obesity analyte signature that has an absence of (e.g., lacks the presence of) 1-methylhistine, glutamine, gamma-amino-n-butyric-acid, isocaproic acid, allo-isoleucine, and hydroxyproline.

In some cases, an obesity analyte signature can include the presence of 1-methylhistine, serotonin, glutamine, gamma-amino-n-butyric-acid, isocaproic acid, allo-isoleucine, alanine, tyrosine, ghrelin, PYY, and includes a HADS-A questionnaire result that indicates anxiety on an anxiety subscale. For example, an obesity phenotype Group 6 (slow burn or low resting energy expenditure) can have an obesity analyte signature that includes the presence of 1-methylhistine, serotonin, glutamine, gamma-amino-n-butyric-acid, isocaproic acid, allo-isoleucine, alanine, tyrosine, ghrelin, PYY, and includes a HADS-A questionnaire result that indicates anxiety on an anxiety subscale. For example, an obesity phenotype Group 6 (slow burn) can have an obesity analyte signature that has an absence of (e.g., lacks the presence of) hydroxyproline, beta-aminoisobutyric-acid, hexanoic acid, and phenylalanine.

In some cases, identifying the obesity phenotype also can include identifying one or more additional variables and/or one or more additional assessments. For example, identifying the obesity phenotype also can include assessing the microbiome of a mammal (e.g., an obese mammal). For example, identifying the obesity phenotype also can include assessing leptin levels. For example, identifying the obesity phenotype also can include assessing the metabolome of a mammal (e.g., an obese mammal). For example, identifying the obesity phenotype also can include assessing the genome of a mammal (e.g., an obese mammal). For example, identifying the obesity phenotype also can include assessing the proteome of a mammal (e.g., an obese mammal). For example, identifying the obesity phenotype also can include assessing the peptidome of a mammal (e.g., an obese mammal).

### Clinical Uses

Once the obesity phenotype of the mammal has been identified, the obesity phenotype can be used to select a treatment option for the mammal. For example, once a mammal is identified as being responsive to one or more interventions (e.g., pharmacological intervention, surgical intervention, weight loss device, diet intervention, lifestyle intervention, behavior intervention, and/or microbiome intervention) based, at least in part, on an obesity phenotype, which is based, at least in part, on an obesity analyte signature in the sample, the mammal can be administered or instructed to self-administer one or more interventions.

Individualized pharmacological interventions for the treatment of obesity (e.g., based on the obesity phenotypes as described herein) can include any one or more (e.g., 1, 2, 3, 4, 5, 6, or more) pharmacotherapies (e.g., individualized pharmacotherapies). A pharmacotherapy can include any appropriate pharmacotherapy. In some cases, a pharmacotherapy can be an obesity pharmacotherapy. In some cases, a pharmacotherapy can be an appetite suppressant. In some cases, a pharmacotherapy can be an anticonvulsant. In some cases, a pharmacotherapy can be a GLP-1 agonist. In some cases, a pharmacotherapy can be an antidepressant. In some cases, a pharmacotherapy can be an opioid antagonist. In some cases, a pharmacotherapy can be a controlled release pharmacotherapy. For example, a controlled release pharmacotherapy can be an extended release (ER) and/or a slow release (SR) pharmacotherapy. In some cases, a pharmacotherapy can be a lipase inhibitor. In some cases, a pharmacotherapy can be a DPP4 inhibitor. In some cases, a pharmacotherapy can be a SGLT2 inhibitor. In some cases, a pharmacotherapy can be a dietary supplement. Examples of pharmacotherapies that can be used in an individualized pharmacological intervention as described herein include, without limitation, orlistat, phentermine, topiramate, lorcaserin, naltrexone, bupropion, liraglutide, exenatide, metformin, pramlitide, Januvia, canagliflozin, dexamphetamines, prebiotics, probiotics, Ginkgo biloba, and combinations thereof. For example, combination pharmacological interventions for the treatment of obesity (e.g., based on the obesity phenotypes as described herein) can include phentermine-topiramate ER, naltrexone-bupropion SR, phentermine-lorcaserin, lorcaserin-liraglutide, and lorcarserin-januvia. In some cases, a pharmacotherapy can be administered as described elsewhere (see, e.g., Sjostrom et al., 1998 Lancet 352:167-72; Hollander et al., 1998 Diabetes Care 21:1288-94; Davidson et al., 1999 JAMA 281:235-42; Gadde et al., 2011 Lancet 377:1341-52; Smith et al., 2010 New Engl. J. Med. 363:245-256; Apovian et al., 2013 Obesity 21:935-43; Pi-Sunyer et al., 2015 New Engl. J. Med. 373:11-22; and Acosta et al., 2015 Clin Gastroenterol Hepatol. 13:2312-9).

When a mammal is identified as having an obesity phenotype that is responsive to treatment with one or more pharmacotherapies, the mammal can be administered or instructed to self-administer one or more pharmacotherapies. In some cases, when a mammal is identified as having a hungry gut (e.g., abnormal satiety) phenotype, based, at least in part, on an obesity analyte signature, the mammal can be administered or instructed to self-administer one or more a GLP-1 agonists (e.g., liraglutide) to treat the obesity.

In some cases, one or more pharmacotherapies described herein can be administered to an obese mammal as a combination therapy with one or more additional agents/therapies used to treat obesity. For example, a combination therapy used to treat an obese mammal (e.g., an obese human) can include administering to the mammal one or more pharmacotherapies described herein and one or more obesity treatments such as weight-loss surgeries (e.g., gastric bypass surgery, laparoscopic adjustable gastric banding (LAGB), biliopancreatic diversion with duodenal switch, and a gastric sleeve), vagal nerve blockade, endoscopic devices (e.g., intragastric balloons or endoliners, magnets), endoscopic sleeve gastroplasty, and/or gastric or duodenal ablations. For example, a combination therapy used to treat an obese mammal (e.g., an obese human) can include administering to the mammal one or more pharmacotherapies described herein and one or more obesity therapies such as exercise modifications (e.g., increased physical activity), dietary modifications (e.g., reduced-calorie diet), behavioral modifications, lifestyle intervention, commercial weight loss programs, wellness programs, and/or wellness devices (e.g. dietary tracking devices and/or physical activity tracking devices). In cases where one or more pharmacotherapies described herein are used in combination with one or more additional agents/therapies used to treat obesity, the one or more additional agents/therapies used to treat obesity can be administered/performed at the same time or independently. For example, the one or more pharmacotherapies described herein can be administered first, and the one or more additional agents/therapies used to treat obesity can be administered/performed second, or vice versa.

### Lifestyle Intervention

In one aspect, provided herein is a method for facilitating weight loss in a subject that comprises identifying the obesity phenotype and then administering a lifestyle intervention to the subject that is individualized to the subject based on the subject's determined obesity phenotype. Such a lifestyle intervention can be referred to as a phenotype-tailored or guided lifestyle intervention or PLI. The PLI can comprise dietary guidance, guidance on physical activity (e.g., exercise regimen), behavioral intervention (e.g., behavioral counseling program) or any combination thereof. In one embodiment, the dietary guidance is a predetermined diet that is tailored to or determined by the obesity phenotype of the subject. In embodiments where the predetermined diet is tailored to the obesity phenotype of the subject, it can be referred to as a phenotype-guided diet or PHENODIET™. In some cases, the PLI can comprise a phenotype-guided diet, guidance on physical activity (e.g., exercise regimen), behavioral intervention (e.g., behavioral counseling program) or any combination thereof. Identification of the obesity phenotype can comprise detecting an intervention responsive obesity analyte signature in a sample obtained from the subject. Detecting an intervention responsive phenotype can be ascertained using the methods provided herein. The weight loss due to prescription or administration of the lifestyle intervention can be a loss of fat mass in the subject as compared to the subject prior to administration of the PLI.

In some cases, administration of the lifestyle intervention (e.g., PLI) or predetermined diet to a subject with an obesity phenotype as described herein prescribed or administered in any of the methods or systems provided herein can result in a change or alteration in the body weight (e.g., total body weight lost (TBWL)) of the subject, waist circumference, the characteristics that define the obesity phenotype of the subject (e.g., calories to fullness (CTF), gastric emptying, resting energy expenditure (REE), indication of anxiety on HADS, etc.) or any combination thereof. The aforementioned alterations can be as compared to the subject prior to administration of the lifestyle intervention (e.g., PLI) or predetermined diet. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of at least 1%, 2%, 3%, 4%, 5% 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80%. In one embodiment, administration of the lifestyle intervention (e.g., PLI) or predetermined diet to a subject determined to possess an obesity phenotype described herein prescribed or administered in any of the methods or systems provided herein can result in a total body weight lost (TBWL) of the subject of from 2% to 8%. In one embodiment, administration of the lifestyle intervention (e.g., PLI) or predetermined diet to a subject determined to possess an obesity phenotype described herein prescribed or administered in any of the methods or systems provided herein can result in a total body weight lost (TBWL) of the subject of at least 3%. In one embodiment, administration of the lifestyle intervention (e.g., PLI) or predetermined diet to a subject determined to possess an obesity phenotype described herein can result in a total body weight lost (TBWL) of the subject of at least 6%. The aforementioned decrease can be as compared to the subject prior to administration of the lifestyle intervention (e.g., PLI) or predetermined diet.

Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of at most 1, 2, 3, 4, 5,6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 99 or 100 kg. Administration of the lifestyle intervention (e.g., PLI) or predetermined diet to a subject determined to possess an obesity phenotype described herein prescribed or administered in any of the methods or systems provided herein can result in a total body weight lost (TBWL) of the subject of from about 3 kg to about 100 kg, about 5 kg to about 100 kg, about 8 kg to about 100 kg, about 10 kg to about 100 kg, about 15 kg to about 100 kg, about 20 kg to about 100 kg, about 30 kg to about 100 kg, about 40 kg to about 100 kg, about 50 kg to about 100 kg, about 60 kg to about 100 kg, about 70 kg to about 100 kg, about 80 kg to about 100 kg, about 90 kg to about 100 kg, about 3 kg to about 90 kg, about 3 kg to about 80 kg, about 3 kg to about 70 kg, about 3 kg to about 60 kg, about 3 kg to about 50 kg, about 3 kg to about 40 kg, about 3 kg to about 30 kg, about 3 kg to about 20 kg, about 3 kg to about 10 kg, about 5 kg to about 90 kg, about 10 kg to about 75 kg, about 15 kg to about 50 kg, about 20 kg to about 40 kg, or about 25 kg to about 30 kg. The aforementioned decrease can be as compared to the subject prior to administration of the lifestyle intervention (e.g., PLI) or predetermined diet. Administration of the lifestyle intervention (e.g., PLI) or predetermined diet to a subject determined to possess an obesity phenotype described herein prescribed or administered in any of the methods or systems provided herein can result in a decrease in the waist circumference of the subject of at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a decrease in the waist circumference of the subject of at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a decrease in the waist circumference of the subject of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a decrease in the waist circumference of the subject of 1%, 2%, 3%, 4%, 5%, 6%, 7%, 8%, 9%, 10%, 11%, 12%, 13%, 14%, 15%, 16%, 17%, 18%, %19%, 20%, 25%, 30%, 35%, 40%, 45%, 50%, 55%, 60%, 65%, 70%, 75% or 80%. Administration of the lifestyle intervention (e.g., PLI) or predetermined diet to a subject determined to possess an obesity phenotype described herein prescribed or administered in any of the methods or systems provided herein can result in a decrease in the waist circumference of the subject of from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80%. The aforementioned decrease can be as compared to the subject prior to administration of the lifestyle intervention (e.g., PLI) or predetermined diet.

Administration of the lifestyle intervention (e.g., PLI) or predetermined diet to a subject determined to possess an obesity phenotype described herein prescribed or administered in any of the methods or systems provided herein can result in a reduction of the waist circumference of the subject of at least 1, 2, 3, 4, 5, 6, 7, 8, 9 or 10 inches. Administration of the lifestyle intervention (e.g., PLI) or predetermined diet to a subject determined to possess an obesity phenotype described herein prescribed or administered in any of the methods or systems provided herein can result in a reduction of the waist circumference of the subject of at most 1, 2, 3, 4, 5, 6, 7, 8, 9 or 10 inches. Administration of the lifestyle intervention (e.g., PLI) or predetermined diet to a subject determined to possess an obesity phenotype described herein prescribed or administered in any of the methods or systems provided herein can result in a reduction of the waist circumference of the subject of about 1, 2, 3, 4, 5, 6, 7, 8, 9 or 10 inches. Administration of the lifestyle intervention (e.g., PLI) or predetermined diet to a subject determined to possess an obesity phenotype described herein prescribed or administered in any of the methods or systems provided herein can result in a reduction of the waist circumference of the subject of from about 1 inches to about 10 inches, about 1 inches to about 9 inches, about 1 inches to about 8 inches, about 1 inches to about 7 inches, about 1 inches to about 6 inches, about 1 inches to about 5 inches, about 1 inches to about 4 inches, about 1 inches to about 3 inches, about 1 inches to about 2 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 9 inches to about 10 inches, about 2 inches to about 9 inches, about 3 inches to about 8 inches, about 4 inches to about 7 inches, or about 5 inches to about 7 inches. The aforementioned decrease can be as compared to the subject prior to administration of the lifestyle intervention (e.g., PLI) or predetermined diet.

In some cases, the change or alteration in the body weight (e.g., total body weight lost (TBWL)) of a subject with an obesity phenotype as described herein, the waist circumference of the subject, the characteristics that define the obesity phenotype of the subject (e.g., calories to fullness (CTF), gastric emptying, resting energy expenditure (REE), indication of anxiety on HADS, etc.) or any combination thereof that results from administration of the lifestyle intervention (e.g., PLI) or predetermined diet in any of the methods or systems provided herein to the subject results persists for a defined or specified amount of time. The aforementioned alterations can be as compared to the subject prior to administration of the lifestyle intervention (e.g., PLI) or predetermined diet. The defined or specified amount of time can be at least 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. The defined or specified amount of time can be at most 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. The defined or specified amount of time can be about 1, 2, 3, 4, 5, 6, 7, 8, 9, 10, 11, 12, 15, 18, 21, 24, 27, 30, 33, 36, 39, 42, 45, 48, 51, 54, 57, 60, 63, 66, 67, 70, 73, 76, 79, 82, 85, 88, 91, 94, 97, 99, 102, 105, 108, 111, 114, 117 or 120 months. In some cases, the defined or specified amount of time can be at least 15, 20, 25, 30, 35, 40, 45, 50, 55, 60, 65 or 70 years.

In one embodiment, the intervention responsive phenotype of the subject is a hungry brain (abnormal satiation) phenotype as provided herein and the subject is administered a lifestyle intervention specific to the hungry brain phenotype. The lifestyle intervention can comprise a predetermined diet, exercise regimen and/or behavioral counseling. As described herein, a subject with a hungry brain phenotype can have a neurological malfunction in the brain that fails to detect fulness signals when enough calories have been consumed, often leaving subjects unable to feel satisfied after eating a meal. In one embodiment, a subject determined to possess a hungry brain phenotype is prescribed or administered a predetermined diet and an exercise regimen.

In some cases, efficacy of the lifestyle intervention (e.g., PLI) or predetermined diet prescribed or administered in any of the methods or systems provided herein can be ascertained by a change or alteration in the body weight (e.g., total body weight lost (TBWL)) of the subject as provided herein, waist circumference, the characteristics that define the hungry brain phenotype or any combination thereof. The aforementioned alterations can be as compared to the subject prior to administration of the lifestyle intervention (e.g., PLI) or predetermined diet. For example, a subject determined to possess a hungry brain phenotype displayed a decrease in the number of calories consumed to reach fullness following administration of the lifestyle intervention (e.g., PLI) or predetermined diet as compared to the number of calories consumed to reach fullness for the same subject prior to administration of the lifestyle intervention (e.g., PLI) or predetermined diet. The number of calories consumed to reach fullness following administration of the lifestyle intervention (e.g., PLI) or predetermined diet described herein can be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the lifestyle intervention (e.g., PLI) or predetermined diet. The number of calories consumed to reach fullness following administration of the lifestyle intervention (e.g., PLI) or predetermined diet described herein can be at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the lifestyle intervention (e.g., PLI) or predetermined diet. The number of calories consumed to reach fullness following administration of the lifestyle intervention (e.g., PLI) or predetermined diet described herein can be about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% of the number of calories consumed to reach fullness for the same subject prior to administration of the lifestyle intervention (e.g., PLI) or predetermined diet. The number of calories consumed to reach fullness following administration of the lifestyle intervention (e.g., PLI) or predetermined diet described herein can be from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% of the number of calories consumed to reach fullness for the same subject prior to administration of the lifestyle intervention (e.g., PLI) or predetermined diet. The number of kcal consumed to reach fullness following administration of the lifestyle intervention (e.g., PLI) or predetermined diet described herein can be reduced by at least 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the lifestyle intervention (e.g., PLI) or predetermined diet. The number of kcal consumed to reach fullness following administration of the lifestyle intervention (e.g., PLI) or predetermined diet described herein can be reduced by at most 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the lifestyle intervention (e.g., PLI) or predetermined diet. The number of kcal consumed to reach fullness following administration of the lifestyle intervention (e.g., PLI) or predetermined diet described herein can be reduced by about 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal consumed to reach fullness for the same subject prior to administration of the lifestyle intervention (e.g., PLI) or predetermined diet. The fullness both pre- and -post lifestyle intervention (e.g., PLI) or predetermined diet administration, the fullness can be a maximal fullness (MTV) or normal or usual fullness (VTF). The ‘maximal’ fullness (MTV) or ‘usual’ fullness (VTF) can be as measured in a nutrient drink test or to mixed meal (solids) in an ad libitum buffet meal.

In some cases, the subject diagnosed or determined to possess a hungry brain phenotype is administered or prescribed a predetermined diet that is designed to increase perceived fullness and decrease calories consumed in each meal. In some cases, the predetermined diet is a volumetric diet. The volumetric diet can be any diet that is based on the idea that the volume of food a subject eats leads to weight loss. When more food is consumed in a volumetric diet, the subject can experience fullness in a psychological sense. The predetermined diet administered to a subject determined to possess a hungry brain phenotype can be any volumetric diet based on the idea of adding high fiber content items to the subject's diet in order to increase the volume and decrease the energy density of the diet consumed by the subject. The addition of high fiber content food items to the subject's diet should result in a considerable reduction in calorie intake.

In some cases, the volumetric predetermined diet administered to a subject determined to possess a hungry brain phenotype is a volumetric diet comprising dietary fiber. The dietary fiber can be increased versus a control. The control can be as compared to a recommended dietary fiber intake for the subject's demographic group. In some cases, the subject is female, and the recommended dietary fiber intake is 21-25 grams of dietary fiber per day. In some cases, the subject is a male, and the recommended dietary fiber intake is 30-38 grams of dietary fiber per day. The increased dietary fiber is represented by a dietary fiber intake of greater than 2, 2.5, 3, 3.5, 4, 4.5, 5, 5.5, 6, 6.5, 7, 7.5, 8, 8.5, 9, 9.5 or 10 g/kcal, where g is grams of food. The increased dietary fiber is represented by a dietary fiber intake of from 2 to 3, from 3 to 4, from 4 to 5, from 5 to 6, from 6-7, from 7 to 8, from 8 to 9 or from 9 to 10 g/kcal, where g is grams of food. In some cases, the increased dietary fiber is represented by a dietary fiber intake of greater than 3.5 g/kcal, wherein g is grams of food. In some cases, the increased dietary fiber is represented by a dietary fiber intake of greater than 4 g/kcal, wherein g is grams of food.

The volumetric predetermined diet administered to a subject determined to possess a hungry brain phenotype is represented by a dietary fiber content above 5, 10, 15, 20, 25, 30, 35, 40, 45 or 50 g/10 MJ, where g is grams of food. The volumetric predetermined diet administered to a subject determined to possess a hungry brain phenotype is represented by a dietary fiber content above 5 to 10 g/10 MJ, 10 to 15 g/10 MJ, 15 to 20 g/10 MJ, 20 to 25 g/10 MJ, 25 to 30 g/10 MJ, 30 to 35 g/10 MJ, 35 to 40 g/10 MJ, 40 to 45 g/10 MJ or 45 to 50 g/10 MJ, where g is grams of food. In some cases, the volumetric predetermined diet administered to a subject determined to possess a hungry brain phenotype is represented by a dietary fiber content above 20 to 45 g/10 MJ or 25 to 45 g/10 MJ, wherein g is grams of food.

In some cases, the volumetric predetermined diet administered to a subject determined to possess a hungry brain phenotype in any of the methods or systems provided herein comprises an energy density less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food. In some cases, the volumetric predetermined diet administered to a subject determined to possess a hungry brain phenotype comprises an energy density between 90 and 115 kcal/100 g, between 90 and 112 kcal/100 g, between 90 and 110 kcal/100 g, between 90 and 105 kcal/100 g or between 90 and 100 kcal/100 g, wherein g is grams of food.

In some cases, the volumetric predetermined diet administered to a subject determined to possess a hungry brain phenotype in any of the methods or systems provided herein is a volumetric diet comprising insoluble fiber food items such as, for example fruit, vegetables, whole grains, and lean protein sources. Any other food item containing insoluble fiber can be used in the volumetric predetermined diet administered to a subject determined to possess a hungry brain phenotype. Exemplary foods comprising insoluble fiber for use in a volumetric diet administered to a subject determined to possess a hungry brain phenotype can include, but are not limited to, wheat bran, wheat germ, oat bran, beans, lentils and legumes of all kinds (kidney, black, garbanzo, edamame, split peas, lima, navy, white, etc.), berries (e.g., blackberries, blueberries, raspberries, strawberries, etc.), whole grains, especially barley, quinoa, sorghum, millet, amaranth, oatmeal and rye, turnips, green peas, okra, dark leafy greens (e.g., spinach), radishes, rutabaga, coconut (grated flakes or flour), cocoa, apples with skin, pears with skin, flaxseeds, avocados, sunflower seeds, potatoes and sweet potatoes, dried apricots, prunes, raisins, dates and figs, almonds, walnuts, 100% whole grain pasta and breads, passionfruit and popcorn.

In some cases, the volumetric predetermined diet administered to a subject determined to possess a hungry brain phenotype in any of the methods or systems provided herein can be a low-calorie diet (e.g., Mayo Clinic diet). A low-calorie diet in reference to a volumetric diet administered to a subject determined to possess a hungry brain phenotype can mean any volumetric diet wherein the total calories consumed by the subject can amount to 500 kcalories (also referred to 500 Calories). In other words, the volumetric diet administered to a subject with a hungry brain obesity phenotype can be a diet whereby a 500-kcal deficit from a subject's measured or calculated basal metabolic rate is created per day when following the prescribed diet.

In some cases, the volumetric predetermined diet administered to a subject determined to possess a hungry brain phenotype in any of the methods or systems provided herein can comprise time restricted eating. The time restricted eating can mean that the subject is instructed to eat 1, 2, 3, 4, or 5 meals per day. The time restricted eating can mean that the subject is instructed to eat 1-2, 2-3, 3-4 or 4-5 meals per day. In one embodiment the time restricted eating means the subject is instructed to eat 1-2 meals per day. Patients with abnormal satiation (hungry brain) do not feel hungry all the time but are unable to manage the number of calories they consume at each meal. Restricting the mealtime should help regulate daily calorie consumption. If the subject with a hungry brain phenotype is still hungry after the meal, the subject may consume a serving of a vegetable or fruit with each meal. The vegetables or fruit servings can be those found in the Mayo Clinic Diet book (pp. 258-261).

In one embodiment, the intervention responsive phenotype of the subject in any of the methods or systems provided herein is a hungry brain phenotype as provided herein and the subject is administered an exercise regimen. The exercise regimen can comprise a goal of at least 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of at most 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of about 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of from 500 to 1000, from 1000 to 2000, from 2000 to 4000, from 2500 to 5000, from 5000 to 7500, from 7500 to 10000, from 10000 to 12500, from 12500 to 15000 or from 15000 to 20000 steps per day. In one embodiment, the exercise regimen comprises a goal of 10,000 steps per day. In one embodiment, the exercise regimen comprises a goal of at least 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of at most 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of about 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of from 15 minutes to 30 minutes, from 30 minutes to 60 minutes, from 60 minutes to 90 minutes, from 90 minutes to 120 minutes, from 120 minutes to 150 minutes, from 150 minutes to 180 minutes or from 180 minutes to 210 minutes of cardiovascular exercise/week. In one embodiment, the exercise regimen comprises a goal of 150 minutes of cardiovascular exercise/week.

Exemplary meals and/or snacks for use in a predetermined diet prescribed or administered to a subject determined to possess a hungry brain phenotype in any of the methods or systems provided herein can be found in Tables 1 and 2.

TABLE 1
Exemplary meals for 1200 calorie menu.

| | Day 1 | Day 2 |
|---|---|---|
| Meal 1 | Hearty turkey chili 1 ½ cups (2 PD, 2V) | Southwestern Frittata-1 serving (P, V) |
| | Ingredients for serving size of 8 | Ingredients for serving size of 2 |
| | 2 cups chopped zucchini | ½ green or red bell pepper, diced |
| | 2 teaspoons olive oil | 4 fresh mushrooms, sliced (about ½ cup) |
| | 1 cup chopped onion | 6 egg whites |
| | 2 cups chopped celery | ½ cup salsa, reserve 2 tablespoons for |
| | 1 cup chopped bell peppers | garnish |
| | 2 teaspoons chopped fresh garlic | 1/2 cup shredded cheddar cheese |
| | 1 pound chopped cooked turkey | — |
| | 1 ½ tablespoons chili powder | 1 large banana (2F) |
| | 1 teaspoon cumin seed | 1 Whole wheat English muffin with |
| | 2 cups diced canned tomatoes, | 1 ½ teaspoon of Almond butter |
| | no-salt-added | (2 C, FT) |
| | 4 cups canned kidney beans, | |
| | rinsed and drained | |
| | 2 cups low-sodium vegetable broth | |
| | 1 teaspoon brown sugar | |
| | — | |
| | ⅙ avocado (FT) | |
| | 15 whole grain crackers (2 C) | |
| | 1 cup halved strawberries (F) | |
| | ½ cup baby carrots (V) | |
| | -OR- | |
| | Brown Rice Chicken Bowl | |
| | adjust recipe to: | |
| | 5 ounces of chicken, 1 ⅓ cup | |
| | of brown rice, ½ of medium | |
| | size avocado, for dressing use | |
| | low-fat Greek yogurt and use | |
| | less water | |
| | (3PD, 4C, 2V, 3 FT) | |
| | 1 large banana (F) | |
| | 1 medium apple (2F) | |
| | 4 celery stalks, ½ cup baby | |
| | carrots (2V) | |
| | 1 cup of 1% or skim milk (PD) | |
| Meal 2 | 2 ounces grilled Asian salmon | 4 ounces Almond crusted chicken |
| | (1PD, 1 FT) **Serving size is | breast |
| | smaller than recipe | Ingredients for serving size of 4 |
| | Ingredients for serving size of 4 | ¾ cup ground almonds |
| | 1 tablespoon sesame oil | ½ cup all-purpose flour |
| | 1 tablespoon reduced-sodium | 1 teaspoon dry thyme |
| | soy sauce | 1 teaspoon onion powder |
| | 1 tablespoon fresh ginger, | 1 teaspoon garlic powder |
| | minced | ½ teaspoon salt |
| | 1 tablespoon rice wine vinegar | ½ teaspoon pepper |
| | 4 salmon fillets, each 4 ounces | ½ cup skim milk |
| | — | 4 boneless, skinless, chicken |
| | ⅔ cup quinoa (2C) | breast, 4 ounces each |
| | Side salad: 2 cups leafy green | 1 tablespoon olive oil |
| | lettuce, ⅓ cup cucumbers, 5 | — |
| | baby tomatoes with 1 tsp. olive | (2 PD, FT) |
| | oil and 1 tsp. balsamic vinegar | 1 cup brown rice with vegetables |
| | (2V, FT) | (2C, V, FT) (see pg. 311) |
| | 1 medium apple (2F) | 1 cup broccoli with garlic and |
| | | lemon (V) |
| | | Ingredients for serving size of 4 |
| | | 4 cups broccoli florets |
| | | 1 teaspoon olive oil |
| | | 1 tablespoon minced garlic |
| | | 1 teaspoon lemon zest |
| | | ¼ teaspoon kosher salt |
| | | ¼ teaspoon ground black pepper |
| | | — |
| | | Side salad: 2 cups leafy green |
| | | lettuce, ⅓ cup cucumbers, 5 baby |
| | | tomatoes with 1 tsp. olive oil and 1 |
| | | tsp. balsamic vinegar (2V, FT) |
| | | 1 small pear (F) |

TABLE 2
Exemplary meals for 1400 calorie menu.

| | Day 1 | Day 2 |
|---|---|---|
| Meal 1 | 5 ounces Chicken parmesan | Southwestern Frittata-2 |
| | (2PD, 2C) | servings (2PD, 2V) ** see |
| | (See pg. 326 of Mayo Clinic Diet) | recipe/ingredients above |
| | ⅓ cup cooked whole-grain pasta | 1 large banana (2F) |
| | with | Whole wheat English muffin |
| | ⅓ cup marinara sauce (C, V) | with 1½ tsp. of Almond butter |
| | ½ cup grilled or baked zucchini | (2 C, FT) |
| | drizzled with 1 tsp. of olive oil | 1 cup of skim milk (PD) |
| | (V, FT) | |
| | ¾ cup orange sections (F) | |
| | 3 small-dried figs (F) | |
| Meal 2 | 4 ounces grilled Asian salmon (2 | 5 ounces Almond crusted |
| | PD, FT) ** see recipe/ingredients | chicken breast (2 PD, FT) ** |
| | above | see recipe/ingredients above |
| | ⅔ cup brown rice (2C) | 1 small whole grain roll (C) |
| | 6 steamed asparagus spears (V) | 1½ tsp. Margarine-like spread |
| | Side salad: 2 cups leafy green | light (FT) |
| | lettuce, ⅓ cup cucumbers, 5 | 6 red-skinned baby potatoes |
| | baby tomatoes with 1 tsp. olive | sauteed in 1 teaspoon olive oil |
| | oil and 1 tsp. balsamic vinegar | (2C, FT) |
| | (2V, FT) | ½ cup baby carrots (V) |
| | 1 medium apple (2 F) | 1 cup broccoli with garlic and |
| | | lemon (V)** see |
| | | recipe/ingredients above |
| | | 1 medium pear (2F) |

In one embodiment, the intervention responsive phenotype of the subject is a hungry gut phenotype as provided herein and the subject is administered a lifestyle intervention (e.g., PLI) or predetermined diet specific to the hungry gut phenotype in any of the methods or systems provided herein. The lifestyle intervention can comprise a predetermined diet, exercise regimen and/or behavioral counseling. As described herein, a subject with a hungry gut phenotype can often experience rapid gastric emptying, which reduces the duration of fullness and makes them hungry frequently during the day. In one embodiment, a subject determined to possess a hungry gut phenotype is prescribed or administered a predetermined diet and an exercise regimen.

In some cases, efficacy of the lifestyle intervention (e.g., PLI) or predetermined diet prescribed or administered in any of the methods or systems provided herein can be ascertained by a change or alteration in the body weight (e.g., total body weight lost (TBWL)) of the subject as provided herein, waist circumference, the characteristics that define the hungry gut phenotype or any combination thereof. The aforementioned alterations can be as compared to the subject prior to administration of the lifestyle intervention (e.g., PLI) or predetermined diet. For example, a subject determined to possess a hungry gut phenotype displayed a decrease in gastric emptying (e.g., gastric half-emptying time (GE t_{1/2})) following administration of the lifestyle intervention as compared to the gastric emptying (e.g., gastric half-emptying time (GE t_{1/2})) for the same subject prior to administration of the lifestyle intervention (e.g., PLI) or predetermined diet. Gastric emptying in a subject determined to possess a hungry gut phenotype following administration of the lifestyle intervention (e.g., PLI) or predetermined diet described herein can be decreased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the lifestyle intervention (e.g., PLI) or predetermined diet. Gastric emptying in a subject determined to possess a hungry gut phenotype following administration of the lifestyle intervention (e.g., PLI) or predetermined diet described herein can be decreased by at most 1%, 2%, 3%, 4%, 5%, 6%, 7%, 8%, 9%,10% l, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the lifestyle intervention (e.g., PLI) or predetermined diet. Gastric emptying in a subject determined to possess a hungry gut phenotype following administration of the lifestyle intervention (e.g., PLI) or predetermined diet described herein can be decreased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the gastric emptying for the same subject prior to administration of the lifestyle intervention (e.g., PLI) or predetermined diet.

Gastric emptying in a subject determined to possess a hungry gut phenotype following administration of the lifestyle intervention (e.g., PLI) or predetermined diet described herein can be decreased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the gastric emptying for the same subject prior to administration of the lifestyle intervention (e.g., PLI) or predetermined diet.

The predetermined diet can be administered or prescribed to a subject possessing a hungry gut phenotype in any of the methods or systems provided herein can be a diet with protein supplementation. The diet with protein supplementation modulate gastrointestinal hormone secretion, decrease gastric emptying, and prolong the duration of fullness. In one embodiment, the diet with protein supplementation comprises a pre-meal protein shake or snack comprising protein. The diet with protein supplementation can be a diet whose protein content is elevated or increased in comparison to a control or reference. The reference can be a Reference Daily Intake (RDI) for a demographic group (i.e., a given species of a given age, weight, and general health condition) that applies to the subject determined to possess the hungry gut phenotype. For example, the RDI can be for an adult who consumes a 2000 calorie per day diet, whereby the diet with protein supplementation can comprise more than the 50 grams of protein recommended. In some cases, the protein in the diet with protein supplementation can be at least 1%, 2%, 3%, 5% 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the hungry gut phenotype. In some cases, the protein in the diet with protein supplementation can be most 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the hungry gut phenotype. In some cases, the protein in the diet with protein supplementation can be about 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the hungry gut phenotype. In some cases, the protein in the diet with protein supplementation can be 1%, 2%, 3%, 5%10%, 15%, 20%, 25%, 30%, 40%, 50%, 60%, 70% or 80% more protein than the protein recommended for a demographic group (i.e., a given species of a given age, weight, and general health condition) that applies to the subject determined to possess the hungry gut phenotype. In some cases, the protein in the diet with protein supplementation can be at least 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the hungry gut phenotype. In some cases, the protein in the diet with protein supplementation can be from 1% to 5%, from 5% to 10%, for 10% to 15%, from 15% to 20%, from 20% to 25%, from 25% to 30%, from 30% to 40%, from 40% to 50%, from 50% to 60%, from 60% to 70% or from 70% to 80% more protein than the protein recommended for a demographic group (i.e., a given species of a given age, weight, and general health condition) that applies to the subject determined to possess the hungry gut phenotype. In some cases, the diet with protein supplementation comprising a pre-meal protein shake or snack comprising protein comprises greater than 0.5, 0.55, 0.6, 0.65, 0.7, 0.75, 0.8, 0.85, 0.9, 0.95 or 1 g/kg of body weight (BW) per day of protein. In some cases, the diet with protein supplementation comprising a pre-meal protein shake or snack comprising protein comprises greater than 0.8 g/kg of body weight (BW) per day of protein. In some cases, the diet with protein supplementation comprising a pre-meal protein shake or snack comprising protein comprises greater than 10%, 11%, 12%, 13%, 14%, 15% 16%, 17%, 18%, 19%, 20%, 21%, 22%, 23%, 24% or 25% of total energy per day derived from protein. In some cases, the diet with protein supplementation comprising a pre-meal protein shake or snack comprising protein comprises greater than 20% of total energy per day derived from protein. In some cases, the diet with protein supplementation comprising a pre-meal protein shake or snack comprising protein comprises greater than 46 grams per day of protein if the subject is female or 56 grams per day if the subject is male.

The protein source in the diet with protein supplementation prescribed or administered to a subject determined to possess a hungry gut phenotype in any of the methods or systems provided herein can comprise one or more foods that are known to be sources of lean or non-lean protein. The term “lean protein” or “lean protein source” as used herein can refer to the USDA definition of a lean protein source. The USDA defines a lean protein source as a protein source having less than 10 grams of total fat (4.5 grams or less from saturated fat) per 3.5 ounce or 100 gram serving. In some cases, lean protein sources can also have fewer than 95 milligrams of cholesterol in a 3.5-ounce or 100 gram serving. Alternatively, a “lean protein” or “a lean protein source” can refer to a protein source that has 2 to 3 grams of fat per ounce. Exemplary sources of lean protein sources for use in a predetermined diet prescribed and/or administered to a subject with a hungry gut phenotype include, but are not limited to, skinless white meat poultry (e.g., chicken or turkey), lean cuts of pork, lean cuts of beef, bison, tofu, tempeh, eggs and egg whites, white fish (e.g., cod, flounder, fluke, halibut, haddock, tilapia, bass, grouper, etc.), sardines, salmon, oysters, shrimp, tuna, farro, cottage cheese (e.g., low-fat), Greek-style yogurt (e.g., plain Greek yogurt), Icelandic-style yogurt, powdered peanut butter, low-fat milk, beans, peas and lentils. Exemplary sources of non-lean protein can for use in a predetermined diet prescribed and/or administered to a subject with a hungry gut phenotype include, but are not limited to, white meat poultry (e.g., chicken or turkey) containing skin, non-lean cuts of pork, non-lean cuts of beef and peanut butter.

In some cases, the diet with protein supplementation can comprise one or more foods that induce production and/or secretion of appetite suppressing hormones and/or inhibit the production and/or secretion of hunger hormones such as, for example, whey protein, guarana, coffee, cocoa, green tea (or extract thereof), yams, alfalfa, grapefruit, grape extract, ginger (or extract thereof), cayenne pepper (or extract thereof), yerba mate, shellfish (source of chitosan), almonds, avocados, apples, eggs, sweet potatoes, umeboshi plums, dark chocolate, tofu, wasabi, oatmeal, vegetable juice, green leafy vegetables (e.g., spinach, kale, Swiss chard, green cabbage, etc.), salmon, cinnamon, skim milk, pickles, pine nuts, natural peanut butter, hoodia gordonii, saffron (or extract thereof) and flaxseeds. Whey protein has been found to increase appetite suppressing hormones and decrease hunger hormones. Thus, the individualized dietary plan can include either a pre-meal high protein shake or healthy high protein snack comprising whey protein.

In some cases, the predetermined diet administered to a subject determined to possess a hungry gut phenotype in any of the methods or systems provided herein can be a low-calorie diet (e.g., Mayo Clinic diet). A low-calorie diet in reference to a high protein diet administered to a subject determined to possess a hungry gut phenotype can mean any high protein diet wherein the total calories consumed by the subject can amount to 500 kcalories (also referred to 500 Calories). In other words, the high protein diet administered to a subject with a hungry gut obesity phenotype can be a diet whereby a 500-kcal deficit from a subject's measured or calculated basal metabolic rate is created per day when following the prescribed diet.

In some cases, the predetermined diet administered to a subject determined to possess a hungry gut phenotype in any of the methods or systems provided herein can comprise time restricted eating. The time restricted eating can mean that the subject is instructed to eat 1, 2, 3, 4, or 5 meals per day. The time restricted eating can mean that the subject is instructed to eat 1-2, 2-3, 2-4, 3-4, 3-5 or 4-5 meals per day. In one embodiment the time restricted eating means the subject is instructed to eat 3-5 meals per day. In some cases, the predetermined diet administered to a subject with a hungry gut phenotype can consume a pre-meal snack. The pre-meal snack can be a high-protein protein shake or high protein snack. The pre-meal snack can be consumed about 15 minutes, 30 minutes, 45 minutes, 60 minutes, 75 minutes, 90 minutes or 120 minutes before the meal. In one embodiment, the pre-meal snack is consumed 30 minutes before a meal. In one embodiment, the pre-meal snack is consumed 60 minutes before a meal. In one embodiment, the pre-meal snack is consumed about 30 to 45 minutes, about 30 to 60 minutes, about 45 to 75 minutes, about 60 to 90 minutes or about 60 to 120 minutes before a meal. The high-protein shake can comprise one or more foods that induce production and/or secretion of appetite suppressing hormones and/or inhibit the production and/or secretion of hunger hormones such as, for example, whey protein. In one embodiment, the high-protein shake comprises whey protein and as few other components as possible. Pre-meal protein supplementation preferably relied on whey protein for its additional nutritional value. Because liquids empty faster than solids, pre-meal protein shakes can be consumed just 30 minutes before meals.

In one embodiment, the intervention responsive phenotype of the subject is a hungry gut phenotype as provided herein and the subject is administered an exercise regimen in any of the methods or systems provided herein. The exercise regimen can comprise a goal of at least 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of at most 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of about 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of from 500 to 1000, from 1000 to 2000, from 2000 to 4000, from 2500 to 5000, from 5000 to 7500, from 7500 to 10000, from 10000 to 12500, from 12500 to 15000 or from 15000 to 20000 steps per day. In one embodiment, the exercise regimen comprises a goal of 10,000 steps per day. In one embodiment, the exercise regimen comprises a goal of at least 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of at most 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of about 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of from 15 minutes to 30 minutes, from 30 minutes to 60 minutes, from 60 minutes to 90 minutes, from 90 minutes to 120 minutes, from 120 minutes to 150 minutes, from 150 minutes to 180 minutes or from 180 minutes to 210 minutes of cardiovascular exercise/week. In one embodiment, the exercise regimen comprises a goal of 150 minutes of cardiovascular exercise/week.

Exemplary meals and/or snacks for use in a predetermined diet prescribed or administered to a subject determined to possess a hungry gut phenotype in any of the methods or systems provided herein can be found in Tables 3-6.

TABLE 3
Exemplary meals for 1200 calorie menu (4V, 3F, 4C, 4PD, 3FT).

| | Day 1 | Day 2 |
|---|---|---|
| Pre-Breakfast | High protein shake (consume | High protein shake |
| High Protein | ½ hour before the meal) | (consume ½ hour before the meal) |
| Shake or Snack | | |
| Breakfast | Southwestern Frittata-1 | ½ cup of oatmeal made with 1 cup |
| | serving (PD, V) **see | water with 2 Tbsp. raisins (C, F) |
| | recipe/ingredients above | |
| | ½ English muffin with 1½ | |
| | tsp. peanut butter (C, FT) | |
| | 1 small apple (F) | |
| Pre-Lunch High | High protein shake | 1 hardboiled egg (PD) |
| Protein Shake or | (consume ½ hour before the | 1 small apple (F) |
| Snack | meal) | (consume 1-hour before meal) |
| Lunch | Hearty turkey chili 1 cup (PD, | ½ Tuna salad sandwich |
| | 1.5 V) **see | 1 slice whole wheat bread (C) |
| | recipe/ingredients above | ½ cup tuna packed in water (PD) |
| | 8 whole grain crackers (C) | 2 tsp. mayonnaise made with olive |
| | 2 small clementine (F) | oil (FT) |
| | | ⅔ Tbsp. sweet pickle relish |
| | | Side salad: 2 cups leafy green |
| | | lettuce, ⅓ cup cucumbers, 5 baby |
| | | tomatoes, 1 tsp. olive oil, 1 tsp. |
| | | balsamic vinegar (2 V, FT) |
| Pre-Dinner High | High protein shake | ½ cup low-fat cottage cheese (PD) |
| Protein Shake or | | ⅓ cup berries (F) |
| Snack | | (consume 1-hour before meal) |
| Dinner | 2 ounces Grilled Asian | 2.5 ounces Almond crusted chicken |
| | Salmon (PD, FT) **See | breast (PD, FT) **see |
| | recipe above; note that | ingredients/recipe above |
| | serving size is smaller than | ½ cup brown rice with vegetables |
| | recipe | (C, .5 V, .5 FT) (see pg. 311) |
| | ⅓ cup quinoa (C) | 1 cup broccoli with garlic and |
| | Side salad: 2 cups leafy green | lemon (V) |
| | lettuce, ⅓ cup cucumbers, 5 | |
| | baby tomatoes, 1 tsp. olive | |
| | oil, 1 tsp. balsamic vinegar (2 | |
| | V, FT) | |

TABLE 4
Exemplary meals for 1400 calorie menu (4 F/V, 4 C, 5PD, 3FT).

| | Day 1 | Day 2 |
|---|---|---|
| Pre-Breakfast High | High protein shake (consume | High protein shake (consume ½ |
| Protein Shake or | ½ hour before the meal) | hour before the meal) |
| Snack | | |
| Breakfast | Southwestern Frittata-1 serving | ½ cup of oatmeal made with 1 |
| | (PD, V) | cup of skim milk or soy milk with |
| | **see ingredients/recipe above | 2 Tbsp. raisins (C, PD, F) |
| Pre-Lunch High | High protein shake (consume | 1 hard-boiled egg (PD) |
| Protein Shake or | ½ hour before the meal) | 1 small apple (F) |
| Snack | | (consume 1-hour before meal) |
| Lunch | Hearty turkey chili 1½ cup | Tuna salad |
| | (2PD, 2 V) | 3 ounces tuna packed in water |
| | **see recipe/ingredients above | (PD), 2 tsp. mayonnaise made |
| | top with ⅙ of an avocado | with olive oil (FT), ⅔ Tbsp. |
| | (FT) | sweet pickle relish |
| | ¾ cup mixed berries (F) | 2 slices whole wheat bread (2C) |
| | | 1 large kiwi (F) |
| | | Side salad: 2 cups leafy green |
| | | lettuce, ⅓ cup cucumbers, 5 |
| | | baby tomatoes, 1 tsp. olive oil, 1 |
| | | tsp. balsamic vinegar (2 V, FT) |
| Pre-Dinner High | High protein shake (consume | ½ cup edamame (PD) (consume |
| Protein Shake or | ½ hour before the meal) | 1-hour before meal) |
| Snack | | |
| Dinner | 2-ounce Grilled Asian Salmon | 3-ounce sliced or shredded |
| | (PD, FT) **see | chicken* on a 6-inch whole |
| | ingredients/recipe above | wheat wrap (PD, V, C, FT) |
| | Side salad: 2 cups leafy green | *Ingredients serving size of 4 |
| | lettuce, ⅓ cup cucumbers, 5 | 12 ounces sliced deli turkey (low- |
| | baby tomatoes, 1 tsp. olive oil, | sodium) |
| | 1 tsp. balsamic vinegar (2 V, | ¼ cup avocado |
| | FT) | ¼ cup salsa |
| | | 2 whole-wheat tortillas (12-inch |
| | | diameter) |
| | | 1 cup shredded green cabbage |
| | | ½ cup thin sliced carrots |
| | | ½ cup sliced tomatoes |
| | | 6 steamed asparagus spears (V) |
| | | ¾ cup mandarin orange sections |
| | | (F) |

TABLE 5
High Protein Snack Options.

1 cup of bean salad (1PD, 1V, 1FT)(1 can (15 ounces) low-sodium garbanzo beans, rinsed
and drained; 1 can (15 ounces) low-sodium black beans, rinsed and drained; 1 medium red
onion, diced; 6 lettuce leaves; ½ cup celery, finely chopped) with vinaigrette (Ingredients
for serving 6: 2 tablespoons balsamic vinegar; ⅓ cup fresh parsley, chopped; 4 garlic
cloves, finely chopped Ground black pepper, to taste; ¼ cup extra-virgin olive oil)
8 ounces plain unsweetened low-fat yogurt
½ cup edamame
1 hard-boiled egg
½ cup of 1% low-fat cottage cheese
1 cup of fat fee plain Greek yogurt

TABLE 6
Suggested high protein shakes or powders.

| High Protein Powder or | | | |
|---|---|---|---|
| Ready-to-drink Shake | Calories | Protein | |
| Jay Robb Vanilla Whey | 110 | 25 g | www.jayrobb.com/product/whey- |
| Protein Isolate | | | protein-isolate/ |
| EAS 100% Whey | 160 | 30 g | eas.com/ |
| Protein | | | |
| Elite 100% Whey | 140 | 25 g | www.dymatize.com/elitewhey |
| Gold Standard 100% | 120 | 24 g | www.optimumnutrition.com/en- |
| Whey Optimum | | | us?code=BUYMORE&utm_sour |
| Nutrition | | | ce=google&utm_medium=cpc&g |
| | | | clid=EAIaIQobChMI94ziwoji7gI |
| | | | VHCmzAB1Atg6QEAAYASAA |
| | | | EgICovD_BwE&gclsrc=aw.ds |
| Vega Essentials | 140 | 20 g | myvega.com/products/vega- |
| | | | essentials?variant=103787580293 |
| | | | 55 |

In one embodiment, the intervention responsive phenotype of the subject is an emotional hunger phenotype as provided herein and the subject is administered a lifestyle intervention (e.g., PLI) or predetermined diet specific to the emotional hunger phenotype in any of the methods or systems provided herein. The lifestyle intervention can comprise a predetermined diet, exercise regimen and/or behavioral counseling. The rational for the predetermined diet administered to the subject with an emotional hunger obesity phenotype is that obesity is related to eating due to stress or emotional reasons. Subjects with this phenotype can receive behavioral counseling. The behavioral counseling can be directed to address the subject eating in reaction to positive or negative emotions, and to acquire various coping mechanisms. In some cases, the behavioral counseling is performed at regular intervals. The regular intervals can be bi-weekly, weekly, month, semi-monthly or yearly. The regular intervals can be adjusted during the course of treatment of the lifestyle intervention (e.g., PLI) or predetermined diet. In some cases, the behavioral counseling entails individual behavioral counseling, cognitive behavioral group therapy or a combination thereof. The behavioral counseling can be administered by a professional licensed in performing behavioral counseling. In some cases, the behavioral counseling can comprise having the subject attend an emotional eating group. The emotional eating group can be based on standard behavioral weight management strategies and can comprise a goal setting each session, an enhancing social support session, encouraging mindful eating session, promoting a physically active lifestyle, and relapse prevention strategies session. The intervention can be tailored for emotional eaters, so the intervention also included emotion regulation strategies, cognitive restructuring, and harm reduction approaches to high risk eating situations. The counseling can also entail health and wellness coaching, learning skills for managing emotional eating, strategies to build resilience, growing self-compassion, and improving happiness, mindful moment activities including expression of gratitude, reflective questioning, identification of strengths and focusing on connecting with positive emotions that can lead to increased social connection, curiosity and desire to learn. The emotional eating group session can last for defined interval such as, for example, 12 weeks.

In some cases, efficacy of the lifestyle intervention (e.g., PLI) or predetermined diet in any of the methods or systems provided herein can be ascertained by a change or alteration in the body weight (e.g., total body weight lost (TBWL)) of the subject as provided herein, waist circumference, the characteristics that define the emotional hunger phenotype or any combination thereof. The aforementioned alterations can be as compared to the subject prior to administration of the lifestyle intervention (e.g., PLI) or predetermined diet. For example, a subject determined to possess an emotional hunger phenotype displayed a change in the hospital anxiety and depression score (HADS) questionnaire following administration of the lifestyle intervention (e.g., PLI) or predetermined diet as compared to the same subject prior to administration of the lifestyle intervention (e.g., PLI) or predetermined diet such that the HAD S questionnaire result does not indicate a positive an anxiety component following administration of the lifestyle intervention (e.g., PLI) or predetermined diet. In some cases, the change in the hospital anxiety and depression score (HADS) questionnaire following administration of the lifestyle intervention (e.g., PLI) or predetermined diet can be a decrease in the score on the HADS questionnaire by about, at most or at least 1, 2, 3 or 4 as compared to the same subject prior to administration of the lifestyle intervention (e.g., PLI) or predetermined diet such that the HADS questionnaire result no longer shows anxiety.

In some cases, the predetermined diet administered to a subject determined to possess an emotional hunger phenotype in any of the methods or systems provided herein can comprise no assignment of specific macronutrient composition. The predetermined diet administered to a subject determined to possess an emotional hunger phenotype can be aimed at reducing the possibility of using food as a coping mechanism. In some cases, the predetermined diet administered to a subject determined to possess an emotional hunger phenotype can be a low-calorie diet (e.g., Mayo Clinic diet). A low-calorie diet in reference to a volumetric diet administered to a subject determined to possess an emotional hunger phenotype can mean any diet wherein the total calories consumed by the subject can amount to 500 kcalories (also referred to 500 Calories). In other words, the diet administered to a subject with an emotional hunger phenotype can be a diet whereby a 500-kcal deficit from a subject's measured or calculated basal metabolic rate is created per day when following the prescribed diet.

In some cases, the predetermined diet administered to a subject determined to possess an emotional hunger phenotype in any of the methods or systems provided herein can comprise time restricted eating. The time restricted eating can mean that the subject is instructed to eat 1, 2, 3, 4, or 5 meals per day. The time restricted eating can mean that the subject is instructed to eat 1-2, 2-3, 2-4, 3-4, 3-5 or 4-5 meals per day. In one embodiment the time restricted eating means the subject is instructed to eat 3 meals per day with no snacks between meals.

In one embodiment, the intervention responsive phenotype of the subject is an emotional hunger phenotype as provided herein and the subject is administered an exercise regimen in any of the methods or systems provided herein. The exercise regimen can comprise a goal of at least 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of at most 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of about 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of from 500 to 1000, from 1000 to 2000, from 2000 to 4000, from 2500 to 5000, from 5000 to 7500, from 7500 to 10000, from 10000 to 12500, from 12500 to 15000 or from 15000 to 20000 steps per day. In one embodiment, the exercise regimen comprises a goal of 10,000 steps per day. In one embodiment, the exercise regimen comprises a goal of at least 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or 210 minutes of cardiovascular exercise/week. In one embodiment, the exercise regimen comprises a goal of at most 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or2d minutes of cardiovascular exercise/week. In one embodiment, the exercise regimen comprises a goal of about 15 minutes, 30 minutes, 45 minutes, 60 minutes, 75 minutes, 90 minutes, 105 minutes, 120 minutes, 135 minutes, 150 minutes, 165 minutes, 180 minutes, 195 minutes or(2C minutes of cardiovascular exercise/week. In one embodiment, the exercise regimen comprises a goal of from 15 minutes to 30 minutes, from 30 minutes to 60 minutes, from 60 minutes to 90 minutes, from 90 minutes to 120 minutes, from 120 minutes to 150 minutes, from 150 minutes to 180 minutes or from 180 minutes to 210 minutes of cardiovascular exercise/week. In one embodiment, the exercise regimen comprises a goal of 150 minutes of cardiovascular exercise/week.

Exemplary meals and/or snacks for use in a predetermined diet prescribed or administered to a subject determined to possess an emotional hunger phenotype in any of the methods or systems provided herein can be found in Tables 7 and 8.

TABLE 7
Exemplary meals for 1200 calorie menu (4V, 3F, 4C, 4PD, 3FT).

| | Day 1 | Day 2 |
|---|---|---|
| Breakfast | Southwestern Frittata-1 serving | ½ cup of oatmeal made with 1 |
| | (PD, V)**see recipe/ingredients | cup of skim milk or soy milk |
| | above | with 2 Tbsp. raisins (C, PD, F) |
| | ½ whole grain English muffin | |
| | with 1½ tsp. of peanut butter (C, | |
| | FT) | |
| | 1 small banana (F) | |
| Lunch | Hearty turkey chili 1 cup (PD, | Tuna salad sandwich |
| | 1.5 V)** see recipe/ingredients | 2 slices whole wheat bread |
| | above | (2C) |
| | 8 whole grain crackers (C) | ½ cup tuna packed in water |
| | 2 small clementine (F) | (PD) |
| | | 2 tsp. mayonnaise made with |
| | | olive oil (FT) |
| | | ⅔ Tbsp. sweet pickle relish |
| | | 1 cup of grapes (F) |
| | | Side salad: 2 cups leafy green |
| | | lettuce, ⅓ cup cucumbers, 5 |
| | | baby tomatoes, 1 tsp. olive oil, |
| | | 1 tsp. balsamic vinegar (2 V, |
| | | FT) |
| Dinner | 2 ounces Grilled Asian Salmon | 2.5 ounces Almond crusted |
| | (PD, FT) *see recipe/ingredients | chicken breast (PD, FT) *see |
| | above; **Serving size is smaller | recipe/ingredients above |
| | than recipe | ½ cup cooked brown rice |
| | ⅔ cup quinoa (2C) | with vegetables (C, V, FT) |
| | 1 small pear (F) | (see pg. 311) |
| | Side salad: 2 cups leafy green | 1 cup broccoli with garlic and |
| | lettuce, ⅓ cup cucumbers, 5 | lemon (V) |
| | baby tomatoes, 1 tsp. olive oil, 1 | ½ cup pineapple cubed (F) |
| | tsp. balsamic vinegar (2V, FT) | |

TABLE 8
Exemplary meals for 1400 calorie menu (4V/F, 5C, 4PD, 3FT).

| | Day 1 | Day 2 |
|---|---|---|
| Breakfast | Southwestern Frittata-1 serving | ½ cup of oatmeal made with 1 |
| | (PD, V) *see recipe/ingredients | cup of skim milk or soy milk |
| | above | with 2 Tbsp. raisins (C, PD, F) |
| | 1 whole grain English muffin | |
| | with 1½ tsp. of peanut butter | |
| | (2C, FT) | |
| | 1 small banana (F) | |
| Lunch | Hearty turkey chili 1½ cup | Tuna salad sandwich |
| | (2PD, 2V) *see | 2 slices whole wheat bread |
| | recipe/ingredients above | (2C) |
| | 8 whole grain crackers (C) | ½ cup tuna packed in water |
| | 3 small clementine (1.5F) | (PD) |
| | | 2 tsp. mayonnaise made with |
| | | olive oil (FT) |
| | | ⅔ Tbsp. sweet pickle relish |
| | | 1½ cup of grapes (F) |
| | | Side salad: 2 cups leafy green |
| | | lettuce, ⅓ cup cucumbers, 5 |
| | | baby tomatoes, 1 tsp. olive oil, |
| | | 1 tsp. balsamic vinegar (2V, |
| | | FT) |
| Dinner | 2 ounces Grilled Asian Salmon | 5 ounces Almond crusted |
| | (PD, FT) *see recipe/ingredients | chicken breast (2PD, 2FT) |
| | above; **Serving size is smaller | *see recipe/ingredients above |
| | than recipe | ½ cup cooked brown rice |
| | ⅔ cup quinoa (2C) | with vegetables (C, V, FT) |
| | 1 medium pear 1.5(F) | (see pg. 311) |
| | Side salad: 2 cups leafy green | 1 cup sliced strawberries (F) |
| | lettuce, ⅓ cup cucumbers, 5 | 1 cup broccoli with garlic and |
| | baby tomatoes, 1 tsp. olive oil, 1 | lemon (V) |
| | tsp. balsamic vinegar (2V, FT) | ½ cup pineapple cubed (F) |

In one embodiment, the intervention responsive phenotype of the subject is a slow burn phenotype as provided herein and the subject is administered a lifestyle intervention (e.g., PLI) or predetermined diet specific to the slow burn phenotype in any of the methods or systems provided herein. The lifestyle intervention can comprise a predetermined diet, exercise regimen and/or behavioral counseling. The rational for the predetermined diet administered to the subject with an emotional hunger obesity phenotype is that obesity for a subject with this obesity phenotype is related to slow metabolic rate coupled with overall low activity. In some cases, subjects determined possess a slow burn phenotype are prescribed a lifestyle intervention that focuses on an exercise regimen and a predetermined diet designed to support said exercise program. For example, subjects with this phenotype can be prescribed physical activity and strength training coupled with a high protein diet comprising high protein post-workout shakes or high-protein snack to help build muscle to burn more calories. The goal here can be to use protein supplementation to maintain muscles while dieting by enhancing thermogenesis and preserving lean mass.

In some cases, efficacy of the lifestyle intervention (e.g., PLI) or predetermined diet in any of the methods or systems provided herein can be ascertained by a change or alteration in the body weight (e.g., total body weight lost (TBWL)) of the subject as provided herein, waist circumference, the characteristics that define the slow burn phenotype (e.g., lean muscle mass and/or REE) or any combination thereof. The aforementioned alterations can be as compared to the subject prior to administration of the lifestyle intervention (e.g., PLI) or predetermined diet. For example, a subject determined to possess a slow burn phenotype displayed an increase in resting energy expenditure ((REE); indirect calorimetry; either % or kcal/day) following administration of the lifestyle intervention (e.g., PLI) or predetermined diet as compared to the REE (either % or kcal/day) for the same subject prior to administration of the lifestyle intervention (e.g., PLI) or predetermined diet. REE in a subject determined to possess a slow burn phenotype following administration of the lifestyle intervention (e.g., PLI) or predetermined diet described herein can be increased by at least 1%, 2%, 3%, 4%, 5%, 6%, 7%, 8%, 9%, 10, 1%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the lifestyle intervention (e.g., PLI) or predetermined diet. REE in a subject determined to possess a slow burn phenotype following administration of the lifestyle intervention (e.g., PLI) or predetermined diet described herein can be increased by at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the lifestyle intervention (e.g., PLI) or predetermined diet. REE in a subject determined to possess a slow burn phenotype following administration of the lifestyle intervention (e.g., PLI) or predetermined diet described herein can be increased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the REE for the same subject prior to administration of the lifestyle intervention (e.g., PLI) or predetermined diet. REE in a subject determined to possess a slow burn phenotype following administration of the lifestyle intervention (e.g., PLI) or predetermined diet described herein can be increased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the REE for the same subject prior to administration of the lifestyle intervention (e.g., PLI) or predetermined diet. In some cases, a subject determined to possess a slow burn phenotype displayed an increase in lean muscle mass following administration of the lifestyle intervention (e.g., PLI) or predetermined diet as compared to the lean muscle mass for the same subject prior to administration of the lifestyle intervention (e.g., PLI) or predetermined diet. Lean muscle mass in a subject determined to possess a slow burn phenotype following administration of the lifestyle intervention (e.g., PLI) or predetermined diet described herein can be increased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 4%5% 50%, 55%, 60%, 65% , 70%, 75% or 80% versus the lean muscle mass for the same subject prior to administration of the lifestyle intervention (e.g., PLI) or predetermined diet. Lean muscle mass in a subject determined to possess a slow burn phenotype following administration of the lifestyle intervention (e.g., PLI) or predetermined diet described herein can be increased by at most 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the lean muscle mass for the same subject prior to administration of the lifestyle intervention (e.g., PLI) or predetermined diet.

Lean muscle mass in a subject determined to possess a slow burn phenotype following administration of the lifestyle intervention described herein can be increased by about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75% or 80% versus the lean muscle mass for the same subject prior to administration of the lifestyle intervention (e.g., PLI) or predetermined diet. Lean muscle mass in a subject determined to possess a slow burn phenotype following administration of the lifestyle intervention (e.g., PLI) or predetermined diet described herein can be increased from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75% or from 75% to 80% versus the lean muscle mass for the same subject prior to administration of the lifestyle intervention (e.g., PLI) or predetermined diet.

The predetermined diet can be administered or prescribed to a subject possessing a slow burn phenotype in any of the methods or systems provided herein can be a diet that comprises a post-workout protein shake or snack comprising protein. The diet can be a diet whose protein content is elevated or increased in comparison to a control or reference. The reference can be a Reference Daily Intake (RDI) for a demographic group (i.e., a given species of a given age, weight, and general health condition) that applies to the subject determined to possess the slow burn phenotype. For example, the RDI can be for an adult who consumes a 2000 calorie per day diet, whereby the diet can comprise more than the 50 grams of protein recommended. In some cases, the protein in the diet can be at least 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the slow burn phenotype. In some cases, the protein in the diet can be most 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the slow burn phenotype. In some cases, the protein in the diet can be about 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the slow burn phenotype. In some cases, the protein in the diet can be 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the slow burn phenotype. In some cases, the protein in the diet can be at least 1%, 2%, 3%, 5%, 10%, 15%, 20%, 25%, 30%, 40%, 50%, 60%, 70% or 80% more protein than the protein recommended for a demographic group (i.e., a given species of a given age, weight, and general health condition) that applies to the subject determined to possess the slow burn phenotype. In some cases, the protein in the diet can be from 1% to 5%, from 5% to 10%, for 10% to 15%, from 15% to 20%, from 20% to 25%, from 25% to 30%, from 30% to 40%, from 40% to 50%, from 50% to 60%, from 60% to 70% or from 70% to 80% more protein than the protein recommended for a demographic group (i.e., a given species of a given age, weight, and general health condition) that applies to the subject determined to possess the slow burn phenotype. In some cases, the diet comprising a pre-meal protein shake or snack comprising protein comprises greater than 0.5, 0.55, 0.6, 0.65, 0.7, 0.75, 0.8, 0.85, 0.9, 0.95 or 1 g/kg of body weight (BW) per day of protein. In some cases, the diet comprising a post-workout protein shake or snack comprising protein comprises greater than 0.8 g/kg of body weight (BW) per day of protein. In some cases, the diet comprising a post-workout protein shake or snack comprising protein comprises greater than 10%, 11%, 12%1, 3%, 14%, 15% 16%, 7%1, 8%1, 19%, 20%, 21%, 22%, 23%, 24% or 25% of total energy per day derived from protein. In some cases, the diet comprising a post-workout protein shake or snack comprising protein comprises greater than 20% of total energy per day derived from protein. In some cases, the diet comprising a post-workout protein shake or snack comprising protein comprises greater than 46 grams per day of protein if the subject is female or 56 grams per day if the subject is male.

The protein source in the diet prescribed or administered to a subject determined to possess a slow burn phenotype in any of the methods or systems provided herein can comprise one or more foods that are known to comprise lean protein. The term “lean protein” or “lean protein source” as used herein can refer to the USDA definition of a lean protein source. The USDA defines a lean protein source as a protein source having less than 10 grams of total fat (4.5 grams or less from saturated fat) per 3.5 ounce or 100 gram serving. In some cases, lean protein sources can also have fewer than 95 milligrams of cholesterol in a 3.5-ounce or 100 gram serving. Alternatively, a “lean protein” or “a lean protein source” can refer to a protein source that has 2 to 3 grams of fat per ounce. Exemplary sources of lean protein sources for use in a predetermined diet prescribed and/or administered to a subject with a slow burn phenotype include, but are not limited to, skinless white meat poultry (e.g., chicken or turkey), lean cuts of pork, lean cuts of beef, bison, tofu, tempeh, eggs and egg whites, white fish (e.g., cod, flounder, fluke, halibut, haddock, tilapia, bass, grouper, etc.), sardines, salmon, oysters, shrimp, tuna, farro, cottage cheese (e.g., low-fat), Greek-style yogurt (e.g., plain Greek yogurt), Icelandic-style yogurt, powdered peanut butter, low-fat milk, beans, peas and lentils.

In some cases, the predetermined diet administered to a subject determined to possess a slow burn phenotype in any of the methods or systems provided herein can be a low-calorie diet (e.g., Mayo Clinic diet). A low-calorie diet in reference to a high protein diet administered to a subject determined to possess a slow burn phenotype can mean any high protein diet wherein the total calories consumed by the subject can amount to 500 kcalories (also referred to 500 Calories). In other words, the high protein diet administered to a subject with a slow burn obesity phenotype can be a diet whereby a 500-kcal deficit from a subject's measured or calculated basal metabolic rate is created per day when following the prescribed diet.

In some cases, the predetermined diet administered to a subject determined to possess a slow burn phenotype in any of the methods or systems provided herein can comprise time restricted eating. The time restricted eating can mean that the subject is instructed to eat 1, 2, 3, 4, or 5 meals per day. The time restricted eating can mean that the subject is instructed to eat 1-2, 2-3, 2-4, 3-4, 3-5 or 4-5 meals per day. In one embodiment the time restricted eating means the subject is instructed to eat 3 meals per day. In some cases, the individualized predetermined diet prescribed or administered to a subject with a slow burn phenotype can further include either a post-workout shake or snack comprising one or more foods known to possess a high content of lean protein. The post-workout shake, or snack can be consumed about 15 minutes, 30 minutes, 45 minutes, 60 minutes, 75 minutes, 90 minutes or 120 minutes after the workout. In one embodiment, the post-workout shake, or snack is consumed 30 minutes after a workout. In one embodiment, the post-workout shake, or snack is consumed 60 minutes after a workout. In one embodiment, the pre-meal snack is consumed about 30 to 45 minutes, about 30 to 60 minutes, about 45 to 75 minutes, about 60 to 90 minutes or about 60 to 120 minutes before a meal. In one embodiment, the shake or snack is recommended to be consumed no longer than 1-hour after workout.

In one embodiment, the intervention responsive phenotype of the subject is a slow burn phenotype as provided herein and the subject is administered an exercise regimen in any of the methods or systems provided herein. The exercise regimen can comprise a goal of at least 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of at most 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of about 500, 1000, 1500, 2000, 2500, 3000, 3500, 4000, 4500, 5000, 5500, 6000, 6500, 7000, 7500, 8000, 8500, 9000, 9500, 10000, 12500, 15000, 17500 or 20000 steps per day. The exercise regimen can comprise a goal of from 500 to 1000, from 1000 to 2000, from 2000 to 4000, from 2500 to 5000, from 5000 to 7500, from 7500 to 10000, from 10000 to 12500, from 12500 to 15000 or from 15000 to 20000 steps per day. In one embodiment, the exercise regimen comprises a goal of 10,000 steps per day. In one embodiment, the exercise regimen comprises a goal of at least 1 session, 2, sessions, 3 sessions, 4 sessions, 5 sessions, 6 sessions or 7 sessions of interval training workouts per week. In one embodiment, the exercise regimen comprises a goal of at most 1 session, 2, sessions, 3 sessions, 4 sessions, 5 sessions, 6 sessions or 7 sessions of interval training workouts per week. In one embodiment, the exercise regimen comprises a goal of 1 session, 2, sessions, 3 sessions, 4 sessions, 5 sessions, 6 sessions or 7 sessions of interval training workouts per week. In one embodiment, the exercise regimen comprises a goal of 3 interval training workout sessions per week. In some cases, each interval training workout comprises 5 minutes of aerobic warm up, 35 minutes of alternating low and high intensity training, and 5 minutes of aerobic cool down.

Exemplary meals and/or snacks for use in a predetermined diet prescribed or administered to a subject determined to possess a slow burn phenotype in any of the methods or systems provided herein can be found in Tables 9 and 10 as well as the high protein snack option found in Table 5 and the high protein shake options found in Table 6.

TABLE 9
Exemplary meals for 1200 calorie menu (4V, 3F, 4C, 4PD, 3FT).

| | Day 1 | Day 2 |
|---|---|---|
| Breakfast | Southwestern Frittata-1 | ½ cup of oatmeal made with 1 cup |
| | serving (PD, V) **see | skim milk or unsweetened soy milk |
| | recipe/ingredients above | with 2 Tbsp. raisins (C, PD, F) |
| | ½ English muffin with 1½ | |
| | tsp. peanut butter (C, FT) | |
| | 1 medium apple (2F) | |
| Lunch | Hearty turkey chili 1 cup (PD, | ½ Tuna salad sandwich |
| | 1.5 V) **see | 2 slice whole wheat bread (2C) |
| | recipe/ingredients above | ½ cup tuna packed in water (PD) |
| | 8 whole grain crackers (C) | 2 tsp. mayonnaise made with olive |
| | 2 small clementine (F) | oil (FT) |
| | | ⅔ Tbsp. sweet pickle relish |
| | | 1½ cup of whole strawberries (F) |
| | | ⅔ cup of green beans (v) |
| Dinner | 2 ounces Grilled Asian | 2.5 ounces Almond crusted chicken |
| | Salmon (PD, FT) **See recipe | breast (PD, FT) **see |
| | above; note that serving size is | ingredients/recipe above |
| | smaller than recipe | ¾ cup brown rice with vegetables |
| | ⅔ cup quinoa (2C) | (C, V, FT) (see pg. 311) |
| | 1 small pear (F) | 1 cup broccoli with garlic and |
| | Side salad: 2 cups leafy green | lemon (V) |
| | lettuce, ⅓ cup cucumbers, 5 | 1 cup of cubed honeydew or |
| | baby tomatoes, 1 tsp. olive oil, | cantaloupe (F) |
| | 1 tsp. balsamic vinegar (2 V, | |
| | FT) | |

**include a high protein shake, snack, or meal no longer than 30 minutes after your workout.

TABLE 10
Exemplary meals for 1400 calorie menu.

| | Day 1 | Day 2 |
|---|---|---|
| Breakfast | Southwestern Frittata-1 serving | ½ cup of oatmeal made with 1 |
| | (PD, V) **see | cup of skim milk or soy milk with |
| | ingredients/recipe above | 2 Tbsp. raisins (C, PD, F) |
| | 1 slice whole grain toast with 1 | 1 hard-boiled egg (PD) |
| | tsp. butter (C, FT) | |
| | 1 medium banana (2F) | |
| Lunch | Hearty turkey chili 1½ cup | Tuna salad sandwich |
| | (2PD, 2 V) | ½ cup tuna packed in water (PD), |
| | **see recipe/ingredients above | 2 tsp. mayonnaise made with |
| | top with ⅙ of an avocado | olive oil (FT), ⅔ Tbsp. sweet |
| | (FT) | pickle relish |
| | 16 whole grain crackers (2C) | 2 slices of whole grain bread (2C) |
| | ¾ cup mixed berries (F) | 1 large kiwi (F) |
| | | ½ cup baby carrots (V) |
| | | 1 cup of 1% or skim milk (PD) |
| Dinner | 4-ounce Grilled Asian Salmon | 4-ounces sliced or shredded |
| | (1½ PD, FT) **see | chicken* on two (2) 6-inch whole |
| | ingredients/recipe above | wheat wrap (1½ PD, 2V, 2C, |
| | ⅔ cup quinoa (2C) | 2FT) |
| | ¾ cup cooked zucchini (V) | *Ingredients serving size of 4 |
| | ¾ cup mixed berries (F) | 12 ounces sliced deli turkey (low- |
| | | sodium) |
| | | ¼ cup avocado |
| | | ¼ cup salsa |
| | | 2 whole-wheat tortillas (12-inch |
| | | diameter) |
| | | 1 cup shredded green cabbage |
| | | ½ cup thin sliced carrots |
| | | ½ cup sliced tomatoes |
| | | 6 steamed asparagus spears (V) |
| | | ¾ cup mandarin orange sections |
| | | (F) |

**include a high protein shake, snack, or meal no longer than 30 minutes after your workout.

### Methods/Systems for Iterative Obesity Phenotyping and Lifestyle Intervention Tailoring

Also provided herein is an iterative method for treating obesity in a subject suffering from obesity that comprises: (a) determining or predicting an obesity phenotype of the subject; (b) administering a first or initial phenotype-guided lifestyle intervention (PLI) based on the obesity phenotype of the subject determined in step (a); (c) assessing an alteration in one or more parameters following administration of the first (PLI) for a defined period of time; (d) administering a second or subsequent PLI based on the assessment of the alteration in the one or more parameters if the assessment indicates that a desired outcome has not been achieved; and (e) repeating the assessing and administrating steps iteratively until the desired outcome for the subject is achieved, thereby treating obesity in the subject. The assessing in step (c) can lead to assigning a subsequent or revised obesity phenotype. The iterative method can be computer implemented. The one or more parameters can be a change in waist circumference, body weight, total body weight lost, satiety, satiation, resting energy expenditure and/or level of depression or anxiety. The defined period of time can be measured in days, weeks, months or years. In some cases, the defined period of time is between 3-5 days or 5-7 days. In some cases, the defined period of time can be at least, at most or exactly 1, 2, 3, 4, 5,6, 7, 8, 9, 10, 11, 12, 13, 14, 15, 16, 18, 19, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks. The defined period of time can be between 2-4 weeks, between 4-6 weeks, between 6-8 weeks, between 8-10 weeks or between 10-12 weeks. In one embodiment, the defined period of time is 12 weeks. The first and/or second PLI can be any of the PLI's provided herein that are specific for the hungry brain, hungry gut, slow burn or emotional eating obesity phenotypes described herein. In some case, the first and second PLI's can be the same. In some cases, the first and second PLI's can be different. In some cases, the first and second PLI's can share the same predetermined diet, exercise regimen and/or behavioral counseling regimen or protocol. The predetermined diet, exercise regimen and/or behavioral counseling regimen or protocol can be any of those provided herein that are specific for either the hungry brain, hungry gut, slow burn or emotional eating obesity phenotypes. The genetic data, non-genetic multi-omic data and/or questionnaire (e.g., HADS-A questionnaire or any other questionnaire designed to measure depression and/or anxiety) results as well as any outcomes measured can be implemented into a regression analysis or a classification machine learning model in order to determine the obesity phenotype in step (a) as well as any subsequent or revised obesity phenotypes from step (c). Assessment of the alterations in the one or more parameters can be in comparison to the values or measurements of the one or more parameters of the subject prior to administration of a PLI.

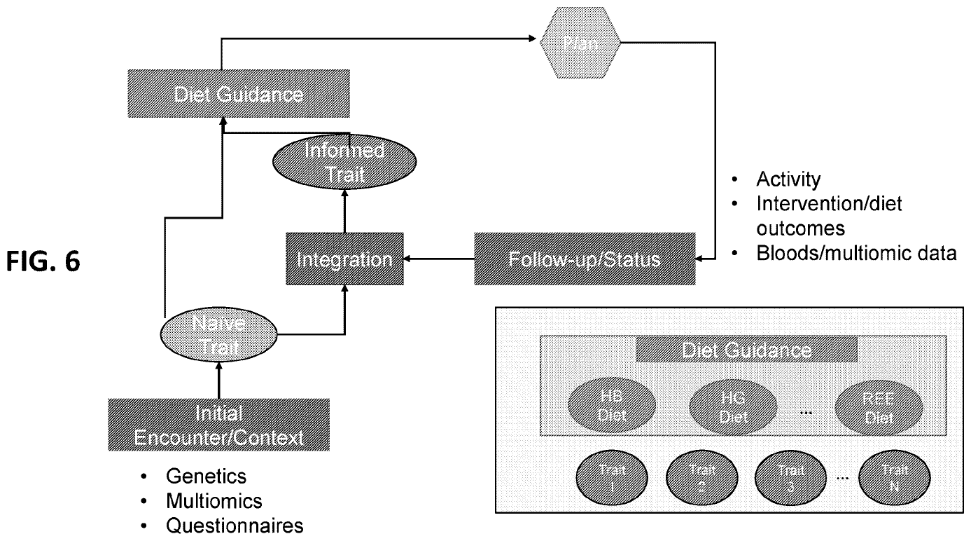

In some cases, the iterative method for treating obesity can entail the methods shown schematically in FIG. 6. In some cases, the methods of FIG. 6 can be implemented on a computer. Further to these embodiments, the method can comprise determining or predicting a naïve trait or obesity phenotype (e.g., soft selection of an obesity phenotype) of a subject suffering from obesity. Determination or prediction of the naïve trait or obesity phenotype (e.g., soft obesity phenotype) can be by measuring a trait known to be associated with an obesity phenotype such as for example, gastric emptying (accelerated gastric emptying and the hungry gut phenotype), calories to fullness at an ad libitum meal (high level of kcals at an ad libitum meal and the hungry brain), resting energy expenditure (low resting energy expenditure and the slow burn phenotype) or an indication of anxiety on a behavioral questionnaire (anxiety result on a HADS-A questionnaire and the emotional eating phenotype). Determination can be by measuring genetic data, non-genetic multi-omic data and/or questionnaire (e.g., HADS-A questionnaire or any other questionnaire designed to measure depression and/or anxiety) results at an initial visit with the subject and/or from a sample obtained (e.g., blood and/or other bodily fluid or tissue sample) from the subject (i.e., initial context in FIG. 6). Following assignment or soft selection of the naïve trait or obesity phenotype, the method can comprise prescribing or administering a first or initial phenotype-guided lifestyle intervention (PLI) that comprises a predetermined diet, exercise regimen and/or behavioral counseling plan such that the first or initial PLI is specific or tailored to the naïve trait or obesity phenotype determined during the initial encounter or context. Following execution of the first or initial PLI by the subject, a follow-up encounter can occur with the subject during which genetic data, non-genetic multi-omic data and/or questionnaire (e.g., HADS-A questionnaire or any other questionnaire designed to measure depression and/or anxiety) results can be measured and/or obtained from the subject and/or from a sample obtained from the subject. During the follow-up encounter, alterations in one or more parameters (e.g., waist circumference, body weight, total body weight lost, satiety, satiation, resting energy expenditure and/or level of depression or anxiety) can also be measured. As shown in FIG. 6, the measurements of the one or more parameters can be combined with the acquired or measured genetic data, non-genetic multi-omic data and/or questionnaire (e.g., HADS-A questionnaire or any other questionnaire designed to measure depression and/or anxiety) results from the follow-up encounter to generate an informed trait or obesity phenotype. In some cases, the informed trait or obesity phenotype can be the same as the naïve trait or obesity phenotype determined during the initial encounter. In some cases, the informed trait or obesity phenotype can be different than the naïve trait or obesity phenotype determined during the initial encounter and can therefore be a revised trait or revised obesity phenotype. Following generation of the informed trait or obesity phenotype, a second or subsequent PLI can be prescribed or administered to the subject if a desired outcome has not been achieved following measurement of the one or more parameters. The second or subsequent PLI can be specific to or tailored to the specific informed trait or obesity phenotype. If the informed trait or obesity phenotype is the same as the naïve trait or obesity phenotype, then the second PLI can be the same as the first PLI. If the informed trait or obesity phenotype is different than the naïve trait or obesity phenotype, then the second PLI can be tailored to the informed trait or obesity phenotype. In some cases, the second PLI can be similar to the first PLI such that only a portion first PLI is altered. The portion can be an alteration of one or two of either the predetermined diet, exercise regimen or behavioral counseling regiment. Additional follow-up visits that can entail measuring alterations in the one or more parameters, genetic data, non-genetic multi-omic data, questionnaire (e.g., HADS-A questionnaire or any other questionnaire designed to measure depression and/or anxiety) results and any combination thereof can be performed to determine subsequent informed traits or obesity phenotypes and subsequent PLI's tailored to said subsequent informed traits or obesity phenotypes can be prescribed or administered until a desired outcome is achieved. The genetic data, non-genetic multi-omic data and/or questionnaire (e.g., HADS-A questionnaire or any other questionnaire designed to measure depression and/or anxiety) results as well as any outcomes measured can be implemented into a regression analysis or a machine learning regression analysis in order to determine the naïve trait or obesity phenotype as well as any subsequent informed traits or obesity phenotypes. Measurement of the alterations in the one or more parameters can be in comparison to the values or measurements of the one or more parameters of the subject prior to administration of a PLI.

Satiety can be determined by measuring gastric emptying. Gastric emptying can be measured using any method known in the art such as, for example, scintigraphy and can be represented as GE T1/4 or GE T1/2. Satiation can be determined by measuring calories to fullness or the amount of calories (e.g., kcal) consumed at an ad libitum meal. Resting energy expenditure can be measured using any method known in the art such as, for example, indirect calorimetry and can be represented as kcal/24 hrs. Anxiety can be measured by completing a behavioral questionnaire designed to assess anxiety (e.g., HADS questionnaire).

In yet another aspect, any method or iterative method provided herein for treating can be a computer implemented method performed by a system. The system can comprise: (a) one or more processors; (b) one or more memories operatively coupled to at least one of the one or more processors and having instructions stored thereon that, when executed by at least one of the one or more processors, cause the system to perform the method or iterative method provided herein for treating obesity; and (c) one or more instruments in communication with at least one of the one or more processors, wherein the instruments, upon receipt of instructions sent by the at least one of the one or more processors, perform the steps of the method or iterative method for treating obesity as provided herein.

In one embodiment, provided herein is a system for determining an obesity phenotype of a subject suffering from obesity, the system comprising: (a) one or more processors; (b) one or more memories operatively coupled to at least one of the one or more processors and having instructions stored thereon that, when executed by at least one of the one or more processors, cause the system to: (i) determine or predict an obesity phenotype of the subject; (ii) prescribe a first or initial phenotype-guided lifestyle intervention (PLI) based on the obesity phenotype of the subject determined in step (i); (iii) assess an alteration in one or more parameters for the subject following administration of the first or initial PLI for a defined period of time; (iv) prescribe a second or subsequent PLI based on the assessment of the alteration in the one or more parameters; and (v) repeat the assessing and prescribing steps iteratively until a desired outcome for the subject is achieved; and (c) one or more instruments in communication with at least one of the one or more processors, wherein the instruments, upon receipt of instructions sent by the at least one of the one or more processors, perform steps (i)-(v).

In one embodiment, provided herein is a system for determining an obesity phenotype of a subject suffering from obesity, the system comprising: (a) one or more processors; (b) one or more memories operatively coupled to at least one of the one or more processors and having instructions stored thereon that, when executed by at least one of the one or more processors, cause the system to: (i) determine or predict a naïve trait of the subject; (ii) prescribe a first diet guidance and plan for the subject based on the naïve trait of the subject determined in step (i); (iii) assess alterations in one or more parameters (e.g., activity, intervention/diet outcomes and genetic and/or non-genetic multi-omic data) of the subject following administration of the diet guidance and plan for a defined period of time; (iv) integrate the alterations in the one or more parameters from step (iii) into a classification machine learning model or a regression analysis to generate an informed trait; (v) prescribe subsequent diet guidance and a plan based on the informed trait generated in step (iv); and (vi) repeat steps (iii)-(v) iteratively until a desired outcome for the subject is achieved; and (c) one or more instruments in communication with at least one of the one or more processors, wherein the instruments, upon receipt of instructions sent by the at least one of the one or more processors, perform steps (i)-(vi).

In one embodiment, the obesity phenotype of the subject is selected from the group consisting of hungry brain (abnormal satiation), hungry gut (abnormal satiety), slow burn (low resting energy expenditure), emotional eating and any combination thereon as described in US20210072259A1.

The obesity phenotype for use in an iterative method provided herein can be determined using any of the methods known in the art (e.g., US20210072259A1) and/or provided herein. For example, the obesity phenotype can be determined using genetic and/or or non-genetic multi-omic data obtained from the subject or a sample obtained from the subject as provided herein. The genetic and/or multi-omic data can be any genetic or multi-omic analytes provided herein. The multi-omic data can be selected from the group consisting of metabolomic data, genomic data, proteomic data, peptidomic data and any combination thereof. In some cases, the multi-omic data obtained for an individual or a sample obtained from an individual represents an obesity analyte signature for that individual. The obesity analyte signature can include any appropriate analyte. Examples of analytes that can be included in an obesity analyte signature described herein include, without limitation, DNA, RNA, proteins, peptides, metabolites, hormones, and exogenous compounds (e.g., medications). In some cases, the obesity analyte signature can be obtained by detecting the presence, absence, or level of one or more metabolites, detecting the presence, or absence, or level one or more peptides (e.g., gastrointestinal peptides), and/or detecting the presence or absence of one or more single nucleotide polymorphisms (SNPs). The DNA, RNA, proteins, peptides, metabolites, hormones, and exogenous compounds (e.g., medications) can be any of those provided herein.

In some cases, an iterative method provided herein is used for subjects suffering from obesity whose obesity phenotype is determined to be a mixture of one or more obesity phenotypes selected from the group consisting of hungry brain, hungry gut, slow burn and emotional eating. In cases where the subject is determined to possess a mixture of obesity phenotypes, the obesity phenotype assigned prior to the assessing step or labeled as the naïve trait can be referred to as the predominant obesity phenotype such that the predominant obesity phenotype is the obesity phenotype that the subject's obesity analyte signature most closely resembles or correlates with.

A regression analysis for use in any methods provided herein can be any regression analysis known in the art and appropriate for the variables (i.e., predictor variable(s) and response variable) utilized in the analysis. In one embodiment, the regression analysis is a ridge regression or least absolute shrinkage and selection operator (LASSO) regression.

In some cases, a classification machine learning model for use in any methods provided herein is selected from the group consisting of Support Vector Classifier (SVC), least absolute shrinkage and selection operator (LASSO) regression, a classification and regression tree (CART) model, and a gradient boosting machine (GBM) model. A regression machine learning model for use in any methods provided herein can be a multi-variate logistic regression, machine learning driven model. In one embodiment, the multi-variate logistic regression, machine learning driven model can utilize a myriad of genetic and non-genetic multi-omic data. The myriad of genetic and non-genetic multi-omic data used in a multi-variate logistic regression machine learning-driven model can be any of the genetic and/or multi-omic data provided herein.

The desired outcome in any of the methods provided herein can be a target level of change or alteration in the one or more parameters in the subject following administration of the first or subsequent PLI or diet guidance and plan as shown in FIG. 6. The one or more parameters can be selected from the group consisting of body weight (e.g., total body weight lost (TBWL)), waist circumference, satiation, satiety, resting energy expenditure, lean muscle mass, anxiety, and any combination thereof. The level of change or alteration in the one or more parameters can be in comparison to the level or value of the one or more parameters in the subject prior to administration of the PLI. The PLI can be the first PLI or the PLI tailored to the naïve trait or initially determined obesity phenotype. The PLI can be a subsequent PLI tailored to the informed trait, or the obesity phenotype determined in a subsequent iteration of an iterative method for treating obesity provided herein. As provided herein, satiety can be determined by measuring gastric emptying. Gastric emptying can be measured using any method known in the art such as, for example, scintigraphy and can be represented as GE T1/4 or GE T1/2. Satiation can be determined by measuring calories to fullness or the amount of calories (e.g., kcal) consumed at an ad libitum meal. Resting energy expenditure can be measured using any method known in the art such as, for example, indirect calorimetry and can be represented as kcal/24 hrs. Anxiety can be measured using a behavioral questionnaire. The behavioral questionnaire can be any questionnaire known in the art for assessing anxiety and/or emotional eating, for example, the Hospital Anxiety and Depression Scale (HADS-A) questionnaire.

In one embodiment, the parameter is satiation, and the target level of change or alteration is a decrease in the number of calories consumed to reach fullness following administration of any PLI (e.g., first PLI or any subsequent PLI) provided herein as compared to the number of calories consumed to reach fullness for the same subject prior to administration of the PLI. The target level of change or alteration in the number of calories consumed to reach fullness following administration of any intervention described herein can be a decrease in the number of calories consumed to reach fullness for the same subject prior to administration of the intervention by about, at most, or at least 5%, 10%, 15%, 20%, 25%, 30%, 35%, 40%, 45%, 50%, 55%, 60%, 65%, 70%, 75% or 80%. The target level of change or alteration in number of calories consumed to reach fullness following administration of any intervention described herein can be a decrease in the number of calories consumed to reach fullness for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%. The target level of change or alteration in number of calories consumed to reach fullness following administration of any intervention described herein can be a decrease in the number of calories consumed to reach fullness for the same subject prior to administration of the intervention by about, at most or at least 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 100 kcal. The fullness both pre- and -post intervention administration can be a maximal fullness (MTV) or normal or usual fullness (VTF). The ‘maximal’ fullness (MTV) or ‘usual’ fullness (VTF) can be as measured in a nutrient drink test or to mixed meal (solids) in an ad libitum buffet meal.

In one embodiment, the parameter is satiety, and the target level of change or alteration is a decrease in the gastric emptying (e.g., gastric half-emptying time (GE t_{1/2})) following administration of any PLI (e.g., first PLI or any subsequent PLI) provided herein as compared to the gastric emptying for the same subject prior to administration of the PLI. The target level of change or alteration in gastric emptying following administration of any intervention described herein can be a decrease in the gastric emptying for the same subject prior to administration of the intervention by about, at most, or at least 5%, 10%, 15%, 20%, 25%, 30%, 35%, 40%, 45%, 50%, 55%, 60%, 65%, 70%, 75% or 80%. The target level of change or alteration in the gastric emptying following administration of any intervention described herein can be a decrease in the gastric emptying for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%.

In one embodiment, the parameter is anxiety, and the target level of change or alteration is a result on a hospital anxiety and depression score (HADS) questionnaire that indicates no anxiety following administration of any PLI (e.g., first PLI or any subsequent PLI) provided herein as compared to the result on the hospital anxiety and depression score (HADS) questionnaire for the same subject prior to administration of the PLI. In some cases, the target level of change on the hospital anxiety and depression score (HADS) questionnaire following administration of any PLI (e.g., first PLI or any subsequent PLI) provided herein can be a decrease in the score on the HADS questionnaire by about, at most or at least 1, 2, 3 or 4 as compared to the same subject prior to administration of the PLI.

In one embodiment, the parameter is resting energy expenditure (REE), and the target level of change or alteration is an increase in the REE (e.g., indirect calorimetry; either % or kcal/day) following administration of any PLI (e.g., first PLI or any subsequent PLI) provided herein as compared to the REE (either % or kcal/day) for the same subject prior to administration of the PLI. The target level of change or alteration in REE following administration of any intervention described herein can be an increase in the REE for the same subject prior to administration of the intervention by about, at most, or at least 5%, 10%, 15%, 20%, 25%, 30%, 35%, 40%, 45%, 50%, 55%, 60%, 65%, 70%, 75% or 80%. The target level of change or alteration in the REE following administration of any intervention described herein can be an increase in the REE for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%.

In one embodiment, the parameter is lean muscle mass, and the target level of change or alteration is an increase in the lean muscle mass following administration of any PLI (e.g., first PLI or any subsequent PLI) provided herein as compared to the lean muscle mass for the same subject prior to administration of the PLI. The target level of change or alteration in lean muscle mass following administration of any intervention described herein can be an increase in the lean muscle for the same subject prior to administration of the intervention by about, at most, or at least 5%10%5, 1%, 20%, 25%, 30%, 35%, 40%, 4%, 50%, 55%, 60%, 65%, 70%, 75% or 80%. The target level of change or alteration in the lean muscle mass following administration of any intervention described herein can be an increase in the lean muscle mass for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%.

In one embodiment, the parameter is body weight, and the target level of change or alteration is a decrease in the body weight (e.g., total body weight lost) following administration of any PLI (e.g., first PLI or any subsequent PLI) provided herein as compared to the body weight (e.g., total body weight lost) for the same subject prior to administration of the PLI. The target level of change or alteration in the body weight (e.g., total body weight lost) following administration of any intervention described herein can be a decrease in the body weight (e.g., total body weight lost) for the same subject prior to administration of the intervention by about, at most, or at least 5%, 10%, 15%, 20%, 25%, 30%, 35%, 40%, 45%, 50%, 55%, 60%, 65%, 70%, 75% or 80%. The target level of change or alteration in the body weight (e.g., total body weight lost) following administration of any intervention described herein can be a decrease in the body weight (e.g., total body weight lost) for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%.

In one embodiment, the parameter is waist circumference, and the target level of change or alteration is a decrease in the waist circumference following administration of any PLI (e.g., first PLI or any subsequent PLI) provided herein as compared to the waist circumference for the same subject prior to administration of the PLI. The target level of change or alteration in the waist circumference following administration of any intervention described herein can be a decrease in the waist circumference for the same subject prior to administration of the intervention by about, at most, or at least 5%, 10%, 15%, 20%, 25%, 30%, 35%, 40%, 45%, 50%, 55%, 60%, 65%, 70%, 75% or 80%. The target level of change or alteration in the waist circumference following administration of any intervention described herein can be a decrease in the waist circumference for the same subject prior to administration of the intervention by 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75% or 75% to 80%.

A computer implemented method described herein may be performed on a single computing machine, a virtual machine, a distributed computing system that includes multiples nodes of computing machines, or any other suitable arrangement of computing devices.

The one or more processors can be one or more computers and coupled together can forma a computer system for implementing any method provided herein.

In some embodiments, the computing system operates as a standalone device or may be connected (e.g., networked) to other machines. In a networked deployment, the machine may operate in die capacity of a server machine or a client machine in a server-client network environment, or as a peer machine in a peer-to-peer (or distributed) network environment.

The structure of the computer system may correspond to any software, hardware, or combined components including but not limited to, a client device, a computing server, and various engines, interfaces, terminals, and machines shown.

By way of example, the computer system may comprise a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions that specify actions to be taken by that machine. Further, the term “machine” and “computer” may also be taken to include any collection of machines that individually or jointly execute instructions to perform any one or more of the methodologies discussed herein.

Instructions can be any directions, commands, or orders that may be stored in different forms, such as equipment-readable instructions, programming instructions including source code, and other communication signals and orders. Instructions may be used in a general sense and are not limited to machine-readable codes.

One and more methods described herein improve the operation speed of the processors used in a system provided herein and reduces the space required for the memory. For example, the machine learning methods described herein can reduce the complexity of the computation of the processors by applying one or more novel techniques that simplify the steps in training, reaching convergence, and generating results of the processors. The algorithms described herein also reduces the size of the models and datasets to reduce the storage space requirement for memory.

The performance of certain of the operations may be distributed among the more than one processors, not only residing within a single machine, but deployed across a number of machines. In some example embodiments, the one or more processors or processor-implemented engines may be located in a single geographic location (e.g., within a home environment, an office environment, or a server farm). In other example embodiments, the one or more processors or processor-implemented engines may be distributed across a number of geographic locations. Even though in the specification or the claims may refer some processes to be performed by a processor, this should be construed to include a joint operation of multiple distributed processors.

The term “computer-readable medium” should be taken to include a single medium or multiple media (e.g., a centralized or distributed database, or associated caches and servers) able to store instructions. The computer-readable medium may include any medium that is capable of storing instructions for execution by the processors and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave and is, thus non-transitory.

### EXAMPLES

The present disclosure is further illustrated by reference to the following Examples. However, it should be noted that these Examples, like the embodiments described above, are illustrative and are not to be construed as restricting the scope of the invention in any way.

### Example 1—Individualized Lifestyle Intervention for Obesity Management

### Background

Obesity is a chronic multifactorial disease associated with multiple metabolic and cardiovascular complications (Heymsfield S B, Wadden T A. Mechanisms, Pathophysiology, and Management of Obesity. N Engl J Med. 2017 Apr. 13; 376(15):1492). The prevalence of obesity and its associated conditions have increased dramatically in recent decades and have become a major concern for most healthcare systems due to their considerable socioeconomic burden (Ward et al. 2019). Even moderate weight loss can lower the risk of cardiovascular disease, improve metabolic parameters, and enhance the quality of life (LeBlanc, E. L., 2018 ‘U.S. Preventive Services Task Force Evidence Syntheses, formerly Systematic Evidence Reviews.’ in, Behavioral and Pharmacotherapy Weight Loss Interventions to Prevent Obesity-Related Morbidity and Mortality in Adults: An Updated Systematic Review for the U.S. Preventive Services Task Force (Agency for Healthcare Research and Quality (US)). Despite advances in our understanding of obesity, the response to weight loss interventions such as lifestyle modifications, pharmacotherapy, endoscopic procedures, and surgeries are still hugely heterogeneous (Bellicha, Alice, Marleen A van Baak, Obesity Reviews: e13256; LeBlanc, E. L., 2018 ‘U.S. Preventive Services Task Force Evidence Syntheses, formerly Systematic Evidence Reviews.’ in, Behavioral and Pharmacotherapy Weight Loss Interventions to Prevent Obesity-Related Morbidity and Mortality in Adults: An Updated Systematic Review for the U.S. Preventive Services Task Force (Agency for Healthcare Research and Quality (US): Rockville (MD)); Khera, Rohan Jama, 315: 2424-34; Hedjoudje, Abdellah, Clinical Gastroenterology and Hepatology, 18: 1043-53. e4; Golzarand, Mahdieh, Surgical endoscopy, 31: 4331-45)

Lifestyle interventions are the cornerstone for weight loss. However, no single diet can be recommended as ideal, and long-term weight loss is not only difficult to attain, but also difficult to maintain; the majority of people who lose weight regain it over time (Tobias, D K, 2015 The lancet Diabetes & endocrinology, 3: 968-79; Sacks, F. 2009 New England Journal of Medicine, 360: 859-73). As a result, it is critical to developing unique lifestyle-intervention strategies that are both successful in lowering weight and easy to implement, increasing the weight loss maintenance. Furthermore, a particular objective of weight management should be health improvement by addressing the neurohormonal, physiological, and behavioral mechanisms that are contributing to obesity.

Since complex physiological feedback mechanisms regulate food intake and body weight and contribute to obesity (Cifuentes, L 2022, Clinics and Research in Hepatology and Gastroenterology, 46: 101794), this should be considered when developing treatment recommendations for obesity. Despite breakthroughs in studying many obesity-contributing factors, they have yet to be translated into meaningful and clinically appropriate dietary advice. Previously, we proposed actionable obesity phenotypes based on pathophysiology and behavior that included calories to fullness (i.e., satiation), gastric motility, and psychological factors (i.e., emotional eating) (Acosta, A., 2015 Gastroenterology, 148: 537-46 e4.). When these phenotypes where targeted, it resulted in higher weight loss with pharmacotherapy after 12 months (Acosta, A. 2021, Obesity (Silver Spring), 29: 662-71).

### Objective

The purpose of the study in this Example was to define an “individualized diet” approach based on obesity related phenotypes (pathophysiology obesity and behavioral classification). The aims of this study were to establish whether a lifestyle intervention based on obesity phenotypes can enhance weight loss outcomes and to determine the effects of the interventions on established obesity.

### Materials and Methods

### Study Design

This study was conducted with approvals from the institutional review board (IRB) at the Mayo Clinic, Rochester. The clinical trial was registered on ClinicalTrials.gov (NCT04073394). The study was conducted according to consensus ethical principles derived from guidelines, including the Declaration of Helsinki and the International Conference on Harmonization Good Clinical Practice Guideline. All participants provided electronic informed consent for study participation and further use of their data. Participants were recruited between July 2020 and September 2021, and data collection was completed in March 2022. From July 2020 to February 2021, participants were assigned to the standard lifestyle intervention (SLI), and from March 2021 to September 2021, participants were assigned to the phenotype-tailored lifestyle intervention (PLI).

The study consisted of a screening period, an in-person physiologic testing visit, and 12 weeks of treatment. Study visits occurred at screening (phenotype testing day), baseline [two-day experience at the Mayo Clinic Healthy Living Program (HLP); day 1-2], at four weeks, and an end visit at week 12 (phenotype testing day). Questionnaires [i.e., Hospital Anxiety and Depression Scale (HADS), Three-Factor Eating Questionnaire (TFEQ-R21), and alcoholism screening test (AUDIT-C)] were completed at home and returned to study staff at the in-person phenotype testing day. Bodyweight, vital signs, and waist and hip circumferences were monitored at every visit. Laboratory parameters were monitored at baseline, week 4, and week 12. Phenotype parameters were measured at baseline and at week 12. Daily caloric intake was assessed with a 24-hour recall of dairy at baseline, week 4, and week 12

### Participants:

### Eligibility Criteria:

Ages Eligible for Study: 18 Years to 65 Years (Adult, Older Adult)
Sexes Eligible for Study: All

### Inclusion Criteria:

Adults with obesity (BMI >30Kg/m2); these were otherwise healthy individuals with no unstable psychiatric disease and uncontrolled life-threatening comorbidities (i.e., unstable angina).
Age: 18-65 years.
Gender: Men or women.

### Exclusion Criteria:

Weight change greater than 3% in the previous 3 months (weight stable).
History of bariatric surgery including lap band and bariatric endoscopy.
Significant untreated psychiatric dysfunction including binge eating disorders and bulimia.
Current use of anti-obesity pharmacotherapy, medications known to affect weight (e.g., corticosteroids) or GLP-1 agonist/analog for type 2 diabetes mellitus.
A positive score on the Alcohol Use Disorders Identification Test (AUDIT-C) questionnaire judged by an investigator.
Patient had a known history of any condition or factor judged by the investigator to preclude participation in the study or which might hinder study adherence.

TABLE 11
Participant Demographics

| | | Phenotype-tailored | |
|---|---|---|---|
| | Standard Lifestyle | lifestyle intervention | |
| | Intervention (SLI) | (PLI) | |
| | N = 81 | N = 84 | p-value |
| Sex, females | 64 (79%) | 70 (83.3%) | 0.48 |
| Age, years | 42.91 (11.59) | 44.77 (12.22) | 0.32 |
| Race, white | 75 (92.5%) | 76 (91.6%) | 0.46 |

The obesity related phenotype of each eligible participant was determined using the methods described in US20210072259A1, which is incorporated herein by reference. In summary, the pathophysiology obesity classification was ascertained by having the eligible participant complete the following validated tests: a) satiation, studied by ad libitum buffet meal (kcal consumed to reach maximal fullness) and visual analog scale for fullness (100 mm scale) at baseline and postprandial every 30 minutes for 2 hours, b) satiety, studied by visual analog scale for appetite (100 mm scale) at baseline and postprandial every 30 minutes for 2 hours after a standard 300 kcal meal and gastric emptying of solids (summarized by the half-emptying time, T1/2, minute), c) hedonic, studied by hospital anxiety and depression score (HADS) and Three Eating Factor Questionnaires, and d) energy expenditure, studied by resting energy expenditure (REE) (indirect calorimetry), non-exercise physical activity and exercise. Then, with the intention to translate the unsupervised obesity-related phenotype principal component analysis (PCA) to quantifiable and reproducible specific apriori determined cutoff, the 75th percentile of the median of each measurement in females and males for each phenotype was used as a cutoff to identify prevalence of the four (4) distinct phenotypes among the study participants.

The phenotypic studies to be utilized in assessing the obesity related phenotypes in this study included (all to be performed on same day in the following order): fasting blood collection, resting energy expenditure, gastric emptying with meal for breakfast, behavioral questionnaires, and buffet meal test for lunch. Blood was collected for assessment of metabolomic biomarkers, gastrointestinal hormones, DNA (blood), and pharmacogenomics. Stool samples were collected for microbiome and bile acid assessment. All participants were contacted at 4 weeks and seen at 12 weeks (current standard in practice). A stool sample and a fasting blood sample were collected at the 12-week visit. At the 12-week visit, participants were unblinded to their “obesity-related phenotype” and were allowed to contact an Obesity Expert physician and/or a registered dietitian nutritionist to continue on an obesity-related phenotype individualized lifestyle plan as part of clinical care. Study team will prospectively follow the patients' weight and waist circumference.

### Anthropometrics and Phenotype Studies

Anthropometrics Measurements were taken of weight, BMI and waist circumference at baseline.

TABLE 12
Anthropometrics

| | SLI | PLI | |
|---|---|---|---|
| | N = 81 | N = 84 | p-value |
| Weight, kg | 109.43 (19.91) | 111.81 (23.74) | 0.49 |
| BMI, kg/m² | 37.97 (6.03) | 38.66 (6.93) | 0.49 |
| Waist Circumference, cm | 115.93 (12.76) | 116.58 (14.57) | 0.76 |

Abbreviations used: BMI, body mass index;
p-value: calculated with t-test or chi-square test as appropriate

Phenotype studies at baseline and week 12: After an 8-hour fasting period, and the following validated quantitative traits (phenotypes) were measured at baseline:

TABLE 13
Quantitative traits (phenotypes) measured at baseline in both groups.

| | | |
|---|---|---|
| SLI | PLI | |
| N = 81 | N = 84 | p-value |

Phenotype Characteristics

| | | | | | |
|---|---|---|---|---|---|
| Calories to fullness, kcal | 924.25 | (371.34) | 859.72 | (329.81) | 0.24 |
| Gastric Emptying T ½, minutes | 131.75 | (33.28) | 127.69 | (34.3) | 0.44 |
| REE, kcal/day | 1889.82 | (315.86) | 1859.2 | (347.9) | 0.56 |
| REE predicted/measured, % | 100.57 | (14.01) | 98.93 | (14.17) | 0.46 |
| HADS Anxiety, score | 4.22 | (3.2) | 4.7 | (3.43) | 0.36 |

Phenotype Distribution

| | | | | | |
|---|---|---|---|---|---|
| Hungry Brain, n | 30 | (37.5%) | 28 | (33.3%) | 0.57 |
| Hungry Gut, n | 7 | (8.6%) | 13 | (15.5%) | 0.17 |
| Emotional Eating, n | 19 | (23.5%) | 26 | (30.9%) | 0.27 |
| Slow Burn, n | 31 | (38.3%) | 37 | (44.6%) | 0.41 |

Abbreviations used: HADS, hospital anxiety and depression scale; REE, Resting energy expenditure.
p-value: calculated with t-test or chi-square test as appropriate

a) The DEXA scan (dual energy x-ray absorptiometry) to measure body composition.

b) Resting energy expenditure was assessed by indirect calorimetry with a ventilated hood.

c) Gastric emptying (GE) of solids by scintigraphy: The primary endpoint was gastric half-emptying time (GE t_{1/2}) as described elsewhere (see, e.g., Acosta et al., 2015 Gastroenterology 148:537-546; Vazquez et al., 2006 Gastroenterology 131:1717-24; and Camilleri et al., 2012 Neurogastroenterology and Motility 24:1076).

d) Appetite (hunger level) was assessed by visual analog score fasting and after standard meal for GE and prior to the Satiation test as described elsewhere (see, e.g., Acosta et al., 2015 Gastroenterology 148:537-546).

e) Satiation was measured by ad-libitum buffet meal to measure total caloric intake and macronutrient distribution in the chosen food. Satiation will be reported in calories consumed at fullness (satiation) as described elsewhere (see, e.g., Acosta et al., 2015 Gastroenterology 148:537-546).

f) Satiety was assessed by visual analog score postprandial after standard meal for GE and after to the ad-libitum meal test for every 30 minutes for 2 hours as described elsewhere (see, e.g., Acosta et al., 2015 Gastroenterology 148:537-546). Satiety was measured in length of time of fullness.

g) Self-administered questionnaires assessing affect, physical activity levels, attitudes, body image, and eating behavior; details of each questionnaire are provided below.

Emotional eating was assessed by Hospital Anxiety and Depression Score (HADS) and Three Factor Eating Questionnaires (TFEQ)

Energy expenditure was studied by REE by indirect calorimetry, reported non-exercise physical activity and reported exercise.

h) Sample collection, handling and storage: Samples were collected after an overnight fast (of at least 8 hours) in the morning. Plasma was preserved following standard guidelines and protein degradation inhibitors, kalikrein and DPP-IV inhibitors were added to preserve the samples. Samples are stored at −80.degree. C.

i) Plasma gastrointestinal hormones (Total and active Ghrelin, GLP-1, CCK, PYY and bile acids) were measured by radioimmunoassay after fasting, and 15, 45, and 90 minutes postprandial, with the primary endpoint being the peak postprandial level (test should be done simultaneously to GE).

j) Targeted Metabolomics: targeted metabolomics of salient classes of compounds in plasma samples were performed using mass spectrometry.

k) Blood DNA: Buccal Swab DNA was collected.

l) Stool was collected and stored to study microbiome, short chain fatty acids, and bile acids.

Studies at 12-Week Visit:

m) Stool and fasting blood sample was collected and stored. Stool was used to measure microbiome, short chain fatty acids and bile acids (as above). Fasting blood was used to GI hormones and metabolomics (as above).

### Questionnaires to Assess Behavior

Participants completed a series of questionnaires: Weight management Questionnaire (Mayo Clinic®), the and the Hospital Anxiety and Depression Inventory [HAD (see, e.g., Zigmond et al., 1983 Acta Psychiatrica Scandinavica 67:361-70)] to appraise the contribution of affective disorder.

Behavioral Questionnaires:

a. AUDIT-C Alcoholism Screening Test—This score was used in screening that can help identify persons who are hazardous drinkers or have active alcohol use disorders.

b. Eating Disorders Questionnaire—The Questionnaire on Eating and Weight Patterns-Revised, is a valid measure of screening for eating disorders which has been used in several national multi-site field trials. Respondents are classified as binge eating disorder, purging bulimia nervosa, non-purging bulimia nervosa, or anorexia nervosa.

c. Body Image Satisfaction—The Multidimensional Body-Self Relations Questionnaire provides a standardized attitudinal assessment of body image, normed from a national body-image survey. Items are rated on a 5-point scale, ranging from 1=Definitely Disagree to 5=Definitely Agree. A sub-scale, the Body Areas Satisfaction Scale, is used to measure feelings of satisfaction with discrete aspects of physical appearance (e.g., face, weight, hair).

d. Eating Behaviors—The Weight Efficacy Life-Style Questionnaire [WEL] is a 20-item eating self-efficacy scale consisting of a total score and five situational factors: negative emotions, availability, social pressure, physical discomfort, and positive activities. Subjects are asked to rate their confidence about being able to successfully resist the urge to eat using a 10-point scale ranging from 0=not confident to 9=very confident.

e. Physical Activity Level—The four-item Physical Activity Stages of Change Questionnaire will be utilized to assess the physical activity level of participants.

f. Three Factor eating questionnaire is a 21-item questionnaire, validated, to assess for emotional eating disorders and food cravings.

g. Hospital Anxiety and Depression Questionnaire (HADS): The HADS is a self-report instrument that contains 14 items on a four-point response scale designed for screening of anxiety and depression.

Control Group: Standard lifestyle intervention (SLI) and Behavioral Treatment

Participants assigned to this group were guided to follow the Mayo Clinic Diet (MCD) book and were recommended to (1) Nutrition: Reduce dietary intake below that required for energy balance by consuming 1200-1500 calories per day for women and 1500-1800 calories per day for men; (2) Physical Activity: reach the goal of 10,000 steps or more per day; (3) Exercise: reach the goal of 150 minutes or more of cardiovascular exercise/week; (4) Limit consumption of liquid calories (i.e. sodas, juices, alcohol, etc.). (5) Behavioral: weekly individual sessions with a wellness coach.

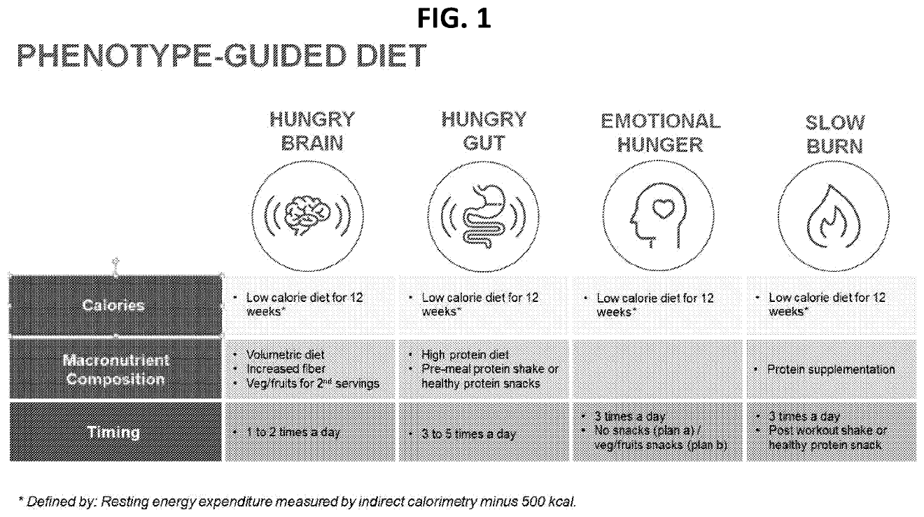

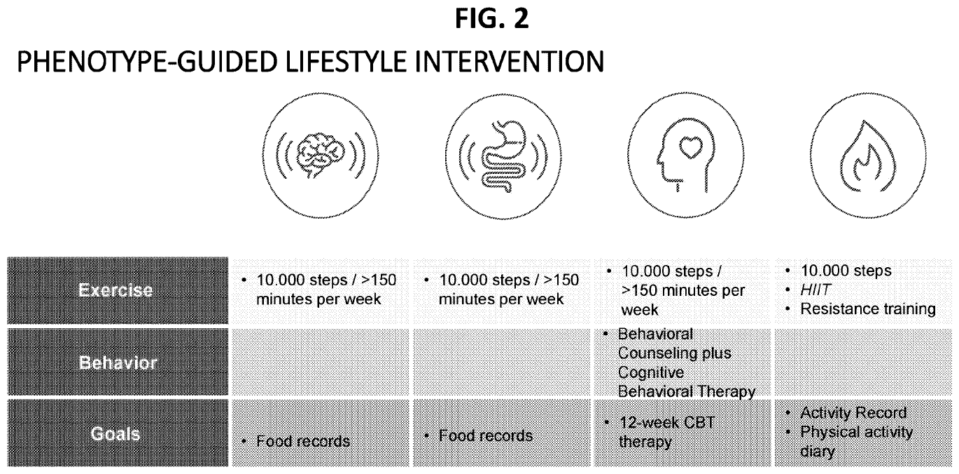

Intervention Group: Phenotype-tailored lifestyle intervention (PLI). As depicted in FIGS. 1 and 2, participants assigned to this group were guided to an individualized dietary and behavioral plan based on the participants obesity phenotype as determined using the methods described herein. In case of a mixed pattern or multiple abnormal phenotypes, the most prominent phenotype was used to tailor the dietary and behavioral or lifestyle advice given. The individualized plans associated with the specific obesity phenotypes were as follows:

### Hungry Brain:

Diet Rationale: Hungry Brain Phenotype is characterized by requiring more calories at each meal to reach fullness (Acosta et al. 2021 Obesity (Silver Spring), 29: 662-71.). These patients might benefit from: i) decreasing their meals' frequency to reduce the daily caloric intake with prolonged fasting periods (time-restricted eating) (Lowe, Wu, 2020 JAMA internal medicine, 180: 1491-99.; Ravussin et al. 2019 Obesity, 27: 1244-54.); ii) increasing dietary non-soluble fiber (volumetric) (Wanders et al. 2011 Obesity Reviews, 12: 724-39; Burton-Freeman 2000 The Journal of nutrition, 130: 272S-75S.); and iii) healthy second servings if needed. The purpose of this abnormal satiation diet was to: i) keep the brain hunger center “off” for longer periods of time (as these patients have normal hunger, but abnormal satiation); ii) produce maximal gastric distention and accommodation to induce the sensation of fullness using a volumetric diet (Wang et al. 2008 Neuroimage, 39: 1824-31.); and iii) recognize the need for second servings and suggest healthy second servings which may also help with reaching satiation (Warrilow et al. 2019 European journal of clinical nutrition, 73: 333-44.).

Food composition: Followed a volumetric diet of insoluble fiber diet including fruit, vegetables, whole grains, and lean protein sources. Increasing the volume and decreasing the energy density by adding high fiber content items should result in a considerable reduction in calorie intake.

Calories: Low calorie diet using the measured resting energy expenditure minus 500 Calories. The term low-calorie diet can refer to creating or following a diet whereby a 500-kcal deficit from a participant's measured or calculated basal metabolic rate is created per day when following the prescribed diet. The recommendation is based on the TOS/ACC/AHA 2013 obesity management recommendations as described in Jensen M D et al., American College of Cardiology/American Heart Association Task Force on Practice Guidelines; Obesity Society. 2013 AHA/ACC/TOS guideline for the management of overweight and obesity in adults: a report of the American College of Cardiology/American Heart Association Task Force on Practice Guidelines and The Obesity Society. Circulation. 2014 Jun. 24; 129(25 Suppl 2):S102-38. doi: 10.1161/01.cir.0000437739.71477.ee. Epub 2013 Nov. 12. Erratum in: Circulation. 2014 Jun. 24; 129(25 Suppl 2).

Time restricted eating: Strongly encouraged 1-2 meals per day. Patients with abnormal satiation do not feel hungry all the time but are unable to manage the number of calories they consume at each meal. Restricting the mealtime should help regulate daily calorie consumption. If the participant was still hungry after the meal, a serving of a vegetable or fruit was recommended to be eaten with the meal. The participant was referred to examples of vegetables and fruit servings in the Mayo Clinic Diet book (pp. 258-261).

All participants were instructed that all calories count, and that it was important to note if fruit or vegetables eaten were above a recommended calorie level.

All participants were instructed to inform the research team if they were struggling to follow the recommended dietary intervention.

Physical Activity: reach the goal of 10,000 steps per day.

Exercise: reach the goal of 150 minutes of cardiovascular exercise/week.

### Hungry Gut:

Diet Rationale: Hungry Gut Phenotype is characterized by having an accelerated gastric emptying and increased postprandial hunger (Gonzalez-Izundegui et al. 2021 Obesity (Silver Spring). 2021 September; 29(9):1497-1507.; Acosta et al. 2021 Obesity (Silver Spring), 29: 662-71.) These patients might benefit from a high-protein diet with protein preloads to increase the early release of gastrointestinal satiety hormones, which have resulted in weight loss and improved satiety long-term (Hutchison et al. 2015 The American journal of clinical nutrition, 102: 1574-84.; Veldhorst et al. 2008 The American journal of clinical nutrition, 90: 519-26.). Also, protein preloads slowed gastric emptying in healthy participants and patients with type 2 diabetes (Ma et al. 2009 Diabetes Care, 32: 1600-2; Pham et al. 2019 Nutrients, 11: 2666.; Watson et al. 2019 Diabetes, Obesity and Metabolism, 21: 930-38).

Food composition: Low-calorie diet with protein supplementation was administered to help modulate gastrointestinal hormone secretion, decrease gastric emptying, and prolong the duration of fullness. Whey protein has been found to increase appetite suppressing hormones and decrease hunger hormones. Thus, the individualized dietary plan included either a pre-meal high protein shake or healthy high protein snack. Pre-meal protein supplementation preferably relied on whey protein for its additional nutritional value. Because liquids empty faster than solids, this was consumed just 30 minutes before meals.

Calories: The individualized dietary intervention was a low-calorie diet (e.g., Mayo Clinic diet) using the measured resting energy expenditure minus 500 calories. As referred to previously herein, the term low-calorie diet refers to creating or following a diet whereby a 500-kcal deficit from a participant's measured or calculated basal metabolic rate is created per day when following the prescribed diet.

Time restricted eating: The participant consumed 3-5 meals per day with a pre-meal high protein shake or healthy high protein snack.

As necessary, shakes were consumed 1%2 hour (30 minutes) before the meal or snacks were consumed 1-hour before the meal.

It was recommended that the protein shake included whey protein and as few other components as possible.

Physical Activity: reach the goal of 10,000 steps per day.

Exercise: reach the goal of 150 minutes of cardiovascular exercise/week.

### Emotional Hunger:

Treatment Plan Rationale: Obesity is related to eating due to stress or emotional reasons. The participants with this phenotype received counseling to address eating in reaction to positive or negative emotions, and to acquire various coping mechanisms. The diet was aimed at reducing the possibility of using food as a coping mechanism. Emotional Eating Phenotype is characterized by negative mood and reward-seeking behaviors in relation to negative and positive emotions. These patients might benefit from a behavioral intervention structured for goal-setting, self-monitoring, and stimulus control (Wadden and Foster 2000 Medical Clinics of North America, 84: 441-61.). Furthermore, mindfulness-based approaches were shown to decrease emotional eating and could help increase self-awareness (Katterman et al. 2014 Eating behaviors, 15: 197-204.).

Food composition: No assignment of specific macronutrient composition will be given.

Calories: The individualized dietary intervention was a low-calorie diet (e.g., Mayo Clinic diet) using the measured resting energy expenditure minus 500 calories. As referred to previously herein, the term low-calorie diet refers to creating or following a diet whereby a 500-kcal deficit from a participant's measured or calculated basal metabolic rate is created per day when following the prescribed diet.

Eating times: The participants consumed three (3) meals per day without snacks.
If the participant was struggling to follow the recommended plan, they were told that it was important to inform the research team.

Physical Activity: reach the goal of 10,000 steps per day.

Exercise: reach the goal of 150 minutes of cardiovascular exercise/week.

Behavioral: The theoretic foundation for the 12 session Emotional Eating group was based on standard behavioral weight management strategies; goal setting each session, enhancing social support, encouraging mindful eating, promoting a physically active lifestyle, and relapse prevention strategies (Brownell and Brownell 2004 The LEARN program for weight management: lifestyle, exercise, attitudes, relationships, nutrition (American Health Publishing Company Euless, TX).) The intervention in this Example was tailored for emotional eaters, so the intervention also included emotion regulation strategies, cognitive restructuring, and harm reduction approaches to high risk eating situations (Schulte et al. 2015 Current psychiatry reports, 17: 1-8.; Himes et al. 2015 Obesity surgery, 25: 922-27.). Health and wellness coaching was included, which has been shown can improve health behaviors (Mettler et al. 2014 American journal of health behavior, 38: 83-91.), improve confidence for health behaviors (Clark et al. 2016) and reduce perceived stress (Clark et al. 2014 Journal of consulting and clinical psychology, 59: 739.). Additionally, to further provide skills for managing emotional eating, we looked to our experiences with our work site wellness programs, including the incorporation of stress management strategies (Werneburg et al. 2011 , Stress and Health, 27: 356-64.), strategies to build resilience, growing self-compassion, and improving happiness. (Berkland et al. 2017 Mayo Clinic Proceedings: Innovations, Quality & Outcomes, 1: 203-10) Mindful moment activities were also practiced with participants and included expression of gratitude, reflective questioning, identification of strengths and focused on connecting participants with positive emotions that lead to increased social connection, curiosity and desire to learn (Fredrickson 2010 Positivity: Groundbreaking research to release your inner optimist and thrive (Simon and Schuster).) See Table 14 for the content of the 12 sessions.

TABLE 14
Emotional Eating Group Program
Content for Weekly Group Sessions

| | |
|---|---|
| 1. | SMART Goal Setting |
| 2. | Self-monitoring |
| 3. | Triggers for Overeating |
| 4. | Thoughts, Feelings and Behaviors |
| 5. | Resiliency and Positive Coping Strategies |
| 6. | Emotional Regulation |
| 7. | Mindfulness and Mindful Eating |
| 8. | Body Positivity and Self-Compassion |
| 9. | Support Network |
| 10. | High Risk Eating Situations |
| 11. | Behavior Chain |
| 12. | Maintaining Motivation for Change |

### Slow Burn:

Diet Rationale: Slow Burn Phenotype is characterized by reduced resting energy expenditure and muscle mass. These patients might benefit from i) a structured exercise plan to increase muscle mass which accounts for most of the overall energy expenditure ratio (Hall et al. 2011) and ii) increasing protein intake. There is an excellent dose-response relationship between resistance training and muscle hypertrophy (Seynnes, de Boer, and Narici 2007 Journal of applied physiology, 102: 368-73.). Moreover, low-carbohydrate, high-protein diets enhanced changes in muscle strength which may contribute to increase energy expenditure (Veldhorst, Westerterp-Plantenga, and Westerterp 2009 The American journal of clinical nutrition, 90: 519-26.; Morton et al. 2018 British journal of sports medicine, 52: 376-84).

Food composition: Participants consumed a low-calorie diet with post-workout high protein shake or snack or meal after workouts.

Calories: The individualized dietary intervention was a low-calorie diet (e.g., Mayo Clinic diet) using the measured resting energy expenditure minus 500 calories. As referred to previously herein, the term low-calorie diet refers to creating or following a diet whereby a 500-kcal deficit from a participant's measured or calculated basal metabolic rate is created per day when following the prescribed diet.

Time restricted eating: Participants will consume three (3) meals per day with a post-workout high protein shake, healthy high protein snack or high protein meal 30 minutes after workout. Shake or snack were recommended to be consumed no longer than 1-hour after workout.

Physical Activity: participants were told to reach the goal of 10,000 steps per day or more and were given access to a home program for mobility and stretching.

Exercise: Emphasis on resistance training and high-intensity interval training (HIIT). Exercise program with online interactive videos incorporating various types of resistance training, such as free weights, resistance bands, and the patient's own body weight, as well as interval training workout sessions (HIIT) in training zones 1 to 5. Participants were instructed to complete at least three (3) sessions of interval training workout per week (5 minutes of aerobic warm up, 35 minutes of alternating low and high intensity training, and 5 minutes of aerobic cool down).

### Statistical Analyses

Data are presented as mean (SD). The primary analyses were conducted on primary analysis cohorts (PA), defined as subjects who had at least one post-baseline assessment. Subjects who did not formally withdraw from the study but did not have a final assessment at 12 weeks were defined as dropouts. The primary approach for missing values due to drop-out or withdrawal in the primary analysis cohort was to use last-value-carried-forward (LVCF). The completers cohort consisting only of subjects who had an assessment at 12 weeks was used for sensitivity analyses. All hypothesis tests were 2-sided with a 0.05 significance level. All effect estimates were be reported with 95% confidence intervals.

The primary endpoint was total body weight loss (TBWL), defined as the change in body weight from baseline to the 12-week time point between groups. Primary and secondary endpoints within phenotypes were also assessed, via an interaction term, to test for differential effects of the phenotype-tailored lifestyle intervention by phenotype. Additional exploratory analyses were undertaken to investigate the mechanisms of weight loss by phenotype. All statistical analyses were performed with JMP®, Version 14.3.0 (SAS Institute Inc., Cary, NC, 1989-2019).

The secondary end points were percentage of responders (defined as number of participants who loss 5% or more of total body weight) compared to baseline in the obesity phenotype guided individualized lifestyle intervention (dietary and lifestyle intervention) group vs. standard of care at four (4) and twelve (12) weeks; percentage of responders with at least 10 and 15% at 12 weeks, and 10% at 6 months and 12 months; percentage of responders at 5%, 10% and 15%; and percentage of responders within each obesity-phenotype group at 4 and 12 weeks. Change in visceral adiposity (DEXA, %); change in phenotype characteristic variables: a) total calories consumed in 24 hours for abnormal satiation; b) calories to fullness in ad libitum meal for abnormal satiation; c) gastric emptying t_{1/2} in minutes for abnormal postprandial satiety; d) gastric emptying % after 120 minutes abnormal postprandial satiety; e) HADS-A score for abnormal emotional eating; d) emotional eating score on the TFEQ for abnormal emotional eating; e) body composition Fat free mass (%) for abnormal energy expenditure; f) kcal/day measured by indirect calorimetry abnormal energy expenditure. Adherence and compliance with program defined by the number of contacts with the team. Improvement in comorbidities in all cohort and per each phenotype.

### Results

From June 2020 to August 2021, 222 participants were screened, and 165 were assigned to treatment: 81 to standard lifestyle intervention (SLI) and 84 to phenotype-tailored lifestyle intervention (PLI). Overall, 146 participants (88.4%) had a 12-week weight assessment, and 118 (⁷1.5%) had a 12-week in-person physiologic testing visit. The proportion of participants non-completing the trial was similar between treatment groups (PLI, 14.3%; SLI, 8.6%; P=0.25). Demographic and baseline clinical characteristics were similar for the 2 groups (Table 11). Most participants were women (81.2%) and white individuals (92%), with a mean (SD) age of 43.9 (11.9) years. Mean body weight was 110.6 (21.9) kg, mean BMI was 38.1 (6.5) kg/m2, and mean waist circumference was 116.3 (13.7) cm. At baseline, 76.8% of participants had 1 or more comorbidities, with depression being the most frequent reported among the participants (32%). Baseline characteristics of participants with the specific phenotype were similar for the 2 groups.

For all patients, mean GE T 12 was 129.7 (33.8) min, mean CTF was 891.2 (351.1) kcal, and mean REE was 1874.3 (331.8) kcal/day. There were no differences between the 2 groups (Table 12). At baseline, 35.4% (n=58) of participants had a hungry brain phenotype; 12.1% (n=20) of participants had a hungry gut phenotype; 27.3% (n=45) of participants had emotional hunger phenotype; 41.4% (n=68) of participants had slow burn phenotype, and 29.6% (n=49) of participants did not have a specific phenotype. There were no differences in the proportion of phenotypes in the 2 groups. And there were no differences in demographic and baseline clinical characteristics between patients with the phenotypes in the 2 groups.

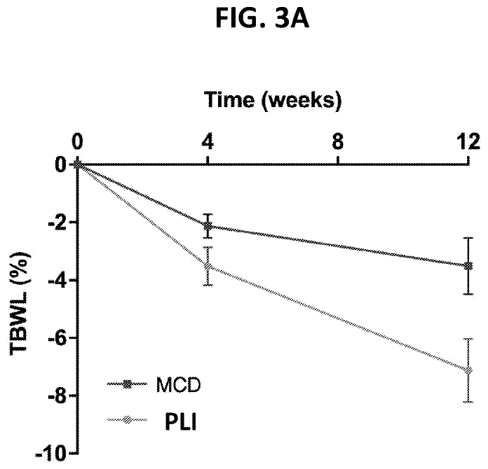

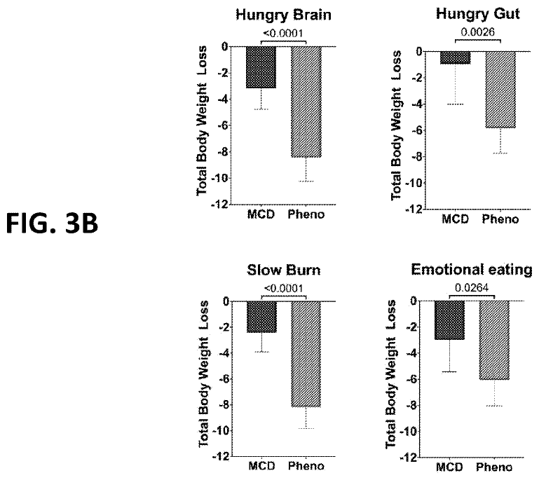

At week 12, the mean weight change from baseline was −7.1% (4.6%) with PLI vs −3.5% (4.2%) with SLI (difference, −3.6 percentage points [95% CI, −5.1 to −2.2]; P<0.001) (Table 15 and FIG. 3A). When analyzing by phenotype, patients with hungry brain phenotype in the PLI group lost −8.2% vs −3.7% in the SLI group (difference, −4.4 percentage points [95% CI, −6.6 to −2.4]; P<0.001). Patients with hungry gut phenotype in the PLI group lost −5.8% (3.0%) vs −0.9% (1.6%) in the SLI group (difference, −4.9 percentage points [95 CI, −7.7 to −2.1]; P=0.002). Patients with slow burn phenotype in the PLI group lost −7.9% (4.7%) vs −2.9% (3.0%) in the SLI group (difference, −5.0 percentage points [95% CI, −7.0 to −2.9]; P<0.001). There was no difference among patients with emotional hunger phenotype and PLI (−3.5% [3.8%]) vs patients with emotional hunger and SLI (−5.4% [4.4%]) (difference, −1.9 percentage points [95 CI, −4.4 to 0.6]; P=0.06) (see FIG. 3B).

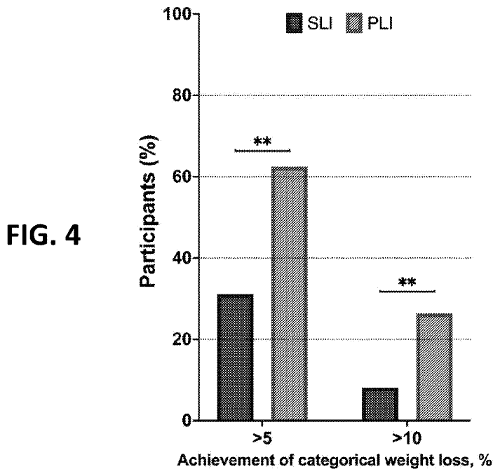

Participants in the PLI group were significantly more likely to have lost at least 5% or 10% of baseline body weight at week 12 vs SLI group (P<0.001 for both). These thresholds were achieved by 62.5% vs 31.1% and by 26.4% vs 8.1% of participants in the PLI and the SLI groups, respectively (FIG. 4). Reductions at week 12 in waist circumference was significantly greater with a PLI than with an SLI (difference, −3.9 cm [95% CI, −6.8 to −1.1]; P<0.001) (Table 15)

TABLE 15
Study outcomes in all patients

| | SLI | | PLI | | |
|---|---|---|---|---|---|
| | Mean Change | | Mean Change | | P-value |
| Weight Loss, % | −3.5 | (4.2) | −7.1 | (4.6) | <0.001 |
| Weight loss, kg | −3.7 | (4.5) | −7.9 | (5.01) | <0.001 |
| Waist circumference, | −4.1 | (7.6) | −8.0 | (7) | <0.001 |
| cm | | | | | |
| Daily caloric intake, | −147.8 | (619.6) | −752.2 | (877.4) | <0.001 |
| kcal | | | | | |
| Calories to fullness, | −95.1 | (248.03) | −127.17 | (198.6) | 0.44 |
| kcal | | | | | |
| Gastric Emptying | −0.66 | (31.6) | 8.2 | (27.4) | 0.11 |
| T ½, | | | | | |
| minutes | | | | | |
| REE, kcal/day | −208.11 | (242.3) | −121.63 | (253.3) | 0.06 |
| REE | −9.18 | (12.4) | −2.3 | (13.6) | 0.008 |
| predicted/measure, % | | | | | |
| Lean mass, % | 1.68 | (1.99) | 2.92 | (2.9) | 0.01 |
| HADS | 0.65 | (2.2) | −0.73 | (2.9) | 0.009 |
| Anxiety, score | | | | | |

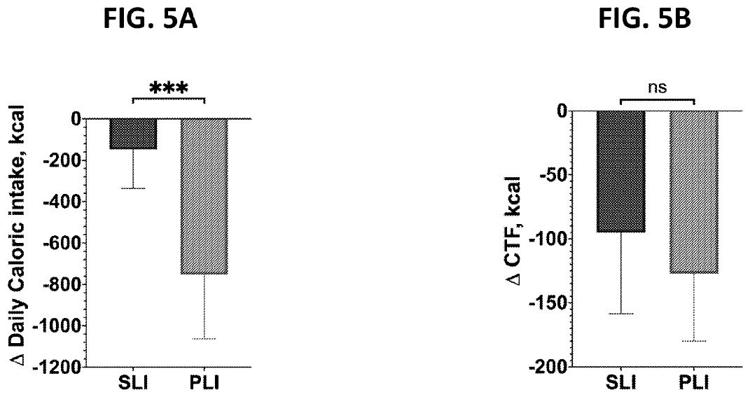

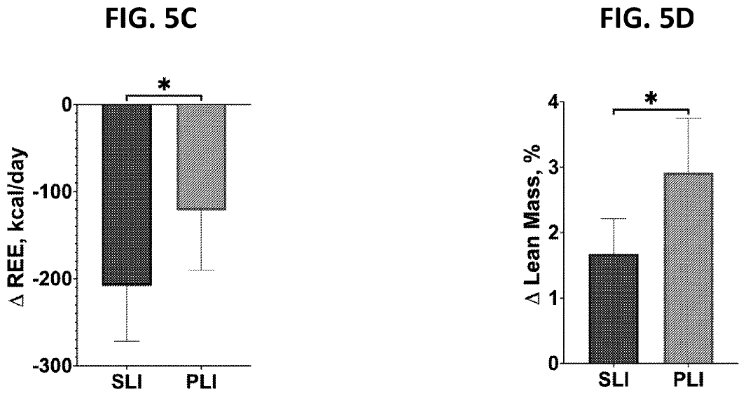

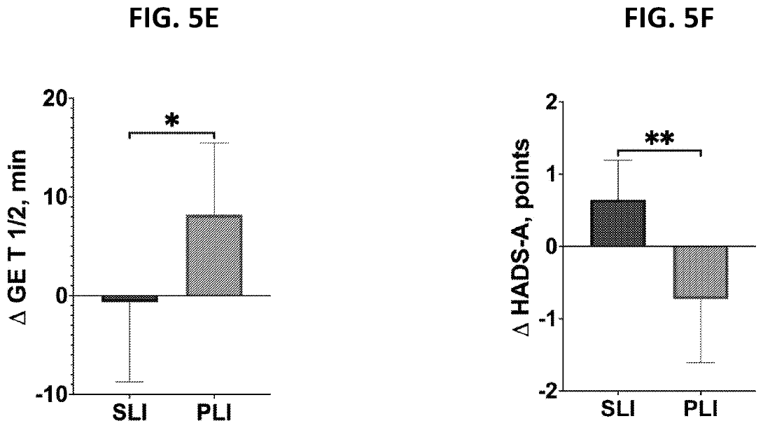

Total daily caloric intake was significantly decreased from baseline in the PLI group (difference, −752.2 kcal/day [95% CI, −1063.3 to −441.1]; P<0.001), but were unchanged in the SLI group (difference, −147.8 kcal/day [95% CI, −336.2 to 40.5]; P=0.12), and this reduction was higher in the PLI group vs the SLI group (P<0.001) (Table 15 and FIG. 5A). CTF in the ad libitum meal was significantly decreased from baseline in the PLI group (difference, −131.5 kcal [95% CI, −184.3 to −78.5]; P<0.001), and in the SLI group (difference, −98.8 kcal [95% CI, −161.9 to to −35.7]; P=0.002); however, this change was not different between groups (P=0.43) (Table 15 and FIG. 5B).

REE was significantly reduced from baseline in the PLI group (difference, −121.6 kcal/day [95% CI, −190.1 to −53.1]; P<0.001) and SLI group (difference, −208.1 kcal/day [95% CI, −271.8 to −144.4]; P<0.001) and this change was not different between groups (P=0.06). Compared with predicted energy expenditure values, measured REE at 12 weeks was significantly decreased from baseline in the SLI group (difference, −9.2% [95% CI, −12.7 to −5.6]; P<0.001) but were unchanged in the PLI group (difference −2.3% [95% CI, −5.9 to 1.4]; P=0.21), and this decreased was groups in the SLI group vs the PLI group (P<0.001) (Table 15 and FIG. 5C).

Lean mass percentage was significantly increased in PLI group (difference, 2.9% [95% CI, 2.1 to 3.7]; P<0.001) and SLI group (difference, 1.7% [95% CI, 1.1 to 2.2]; P<0.001), and this increase was higher in the PLI group (P=0.01) (Table 15 and FIG. 5D).

GE T 1/2 was significantly deaccelerated from baseline in the PLI group (difference, +8.2 min [95% CI, 0.93 to 8.2]; P=0.02), but was unchanged in the SLI group (difference, −0.65 min [95% CI, −8.7 to 7.4]; P=0.87), however, this change was not different between groups (P=0.10) (Table 15 and FIG. 5E)

Anxiety levels (measured by the anxiety component of the HADS) worsened in the SLI group (difference, +0.6 points [95% CI, 0.1 to 1.2]; P=0.02), but were unchanged in the PLI group (difference, −0.7 points [95% CI, −1.6 to 0.2]; P=0.10), and this increase was higher in the SLI group (P<0.001) (Table 15 and FIG. 5F).

Relative to definitory variables for phenotype, participants with hungry brain in the PLI group showed a more significant decrease their daily caloric intake (PLI group mean change, −783 kcal [932] vs. SLI group mean change, 87.6 kcal [621]; P=0.006). Participants with hungry gut did not have any difference in the change in GE T1/2 between the PLI group and the SLI group. Participants with slow burn in the PLI group showed a more significant increase in the lean mass percentage (PLI group mean change, +3.63% [2.31] vs. SLI group mean change, +1.54% [1.72]; P=0.003). Participants with emotional eating in the PLI group showed a more significant decrease in the HADS-anxiety score (PLI group mean change, −3 points [2.4] vs. SLI group mean change, +0.8 points [2.29]; P<. 0.001).

TABLE 16
Outcomes by phenotype groups

| | SLI | | PLI | | |
|---|---|---|---|---|---|
| | Mean Change | | Mean Change | | P value |
| Hungry Brain | | | | | |
| Weight change, % | −3.18 | (4.17) | −8.43 | (4.48) | <.001 |
| Daily caloric intake, kcal | 87.6 | (621) | −783 | (932) | 0.006 |
| Calories to fullness, kcal | −197.18 | (346.65) | −113.47 | (216.57) | 0.45 |
| Hungry Gut | | | | | |
| Weight change, % | 0.14 | (1.66) | −5.82 | (3.02) | <.001 |
| Daily caloric intake, kcal | −294.88 | (425) | −496.3 | (1074) | 0.63 |
| Gastric Emptying T ½, min | 18.67 | (29.5) | 12.74 | (15.43) | 0.66 |
| Slow Burn | | | | | |
| Weight change, % | −2.42 | (3.73) | −8.17 | (4.61) | <.001 |
| Daily caloric intake, kcal | −3.44 | (682) | −772.35 | (614) | 0.003 |
| REE, kcal/day | −25.48 | (285.7) | 6.04 | (265.48) | 0.73 |
| REE predicted/measured, % | 1.5 | (15.57) | 5.47 | (11.91) | 0.40 |
| Lean mass, % | 1.54 | (1,72) | 3.63 | (2.31) | 0.003 |
| Emotional Eating | | | | | |
| Weight change, % | −2.98 | (4.75) | −6.06 | (4.31) | 0.04 |
| Daily caloric intake, kcal | −278.3 | (650) | −695.4 | (935) | 0.26 |
| HADS Anxiety, score | 0.83 | (2.29) | −3 | (2.48) | <.001 |

### Example 2—Iterative System for Individualized Lifestyle Interventions for Obesity Management

### Background

As described herein, the phenotype guided lifestyle intervention approach outlines a phenotype-based selection of a lifestyle (e.g., diet, physical activity and/or behavioral therapy) intervention that improves weight loss efficacy over approaches that don't entail determining a subject's obesity phenotype. However, making a discrete obesity phenotype call may pose limitations that can include: (1) obscuring relevant information about other obesity phenotypes for the subject that may influence diet efficacy; (2) laborious testing to determine the obesity phenotype; and (3) a single point-in-time determination of the subject's obesity phenotype without the ability to adapt to outcomes and biological changes.

### Objective

The purpose of this example is to further extend the PLI approach described throughout the specification and in Example 1 using specific innovations to enhance PLI selection and refine it over time.

### Methods

The methods that will be employed in this example are schematically shown in FIG. 6. As shown in FIG. 6, a subject suffering from obesity will have an initial visit with the study where the obesity phenotype of said subject will be determined utilizing all of the trait data that underlies obesity phenotype selection to make a ‘soft’ selection of the obesity phenotypes, ranking obesity phenotypes by the strength of the underlying trait information, and the corresponding phenotype-guided lifestyle intervention (PLI) for the strongest phenotype (i.e., combination of diet guidance and plan in FIG. 6). The obesity phenotype will be determined using the methods described in US20210072259A1, which is herein incorporated by reference in its entirety. In summary, determination of the subject's predominant obesity phenotype at the initial encounter with the study team will include an assessment of the subject's resting energy expenditure, gastric emptying, calories to fullness, performance on behavioral questionnaire's such as HADS-A in order to assess presence/level of anxiety, as well as performing multi-omic and genetic assessment of samples obtained from the subject in order to analyze the presence, absence and/or levels of discrete analytes correlated with obesity (e.g., select SNPs, gastrointestinal peptides, amino acids, metabolites, etc.). The obesity phenotype assigned to the subject based on the initial encounter will be referred to as the naïve trait and will be used to assign or prescribe an initial phenotype-guided lifestyle intervention (PLI) that will include a dietary, behavioral counseling and physical activity regimen specific to the subject's determined obesity phenotype as provided throughout the specification. It should be noted that the multi-omic, genetic and questionnaire data for the subject will be inputted into a multi-omic regression analysis without making a discrete obesity phenotype call initially, but for later use in the process. Use of the PLI will be prescribed for a defined period of time (e.g., 12-weeks) followed by a subsequent visit to the study team.

At the subsequent visit with the study team, adherence to the PLI (i.e., diet guidance and plan) by the subject will be measured as well as outcomes (e.g., changes in total body weight, waist circumference, resting energy expenditure, calories to fullness, gastric emptying, total daily caloric intake, lean mass percentage, level of anxiety, etc.), levels of activity, and follow-up multi-omic measurements. All of the metrics measured at the subsequent visit will then be integrated into the multi-omic-based regression along with the multi-omic, genetic and questionnaire data collected for the subject at the initial visit to extend the multi-omic regression, revise the “soft” selection of obesity phenotyping by updating the ranking of phenotypes by strength of the underlying trait information, and determine whether or not a revision in diet and/or activity and/or behavioral counseling is needed. This process will be repeated until a desired outcome (e.g., target amount of total body weight lost, target waist circumference, etc.) is reached.

### Numbered Embodiments of the Disclosure

Other subject matter contemplated by the present disclosure is set out in the following numbered embodiments:

1. A method of treating obesity in a subject in need thereof, the method comprising: (a) identifying an obesity phenotype of a subject by detecting an intervention responsive obesity analyte signature in a sample obtained from a subject; and (b) administering a predetermined lifestyle and behavioral plan to the subject, wherein the predetermined lifestyle and behavioral plan is individualized to the obesity phenotype of the subject, thereby treating obesity in the subject.

2. The method of embodiment 1, wherein the administration of the predetermined diet results in weight loss, wherein at least part of the weight loss is due to a loss of fat mass.

3. The method of embodiment 1 or 2, wherein the administration of the predetermined diet is effective to reduce the total body weight of the subject by at least 4%.

4. The method of any one of the above embodiments, wherein the administration of the predetermined diet is effective to reduce the total body weight of the subject by 2% to 8%.

5. The method of any one of the above embodiments, wherein the administration of the predetermined diet is effective to reduce the waist circumference of the subject by from about 1 inches to about 10 inches.

6. The method of any one of the above embodiments, wherein determining the obesity phenotype of the subject further comprises obtaining results from a Hospital Anxiety and Depression Scale (HADS) questionnaire.

7. The method of any one of the above embodiments, wherein the detecting the intervention responsive obesity analyte signature comprises detecting the presence, absence, or level of one or more metabolites, detecting the presence, absence, or level of one or more peptides, and/or detecting the presence, absence, or level of one or more single nucleotide polymorphisms (SNPs) in the sample obtained from the subject.

8. The method of any one of the above embodiments, wherein the sample is selected from the group consisting of a blood sample, a saliva sample, a urine sample, a breath sample, and a stool sample.

9. The method of any one of the above embodiments, wherein the obesity analyte signature comprises 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, ghrelin, peptide tyrosine tyrosine (PYY), GLP-1 and phenylalanine.

10. The method of any one of embodiments 1-8, wherein the obesity analyte signature comprises a presence of HTR2C, GNB3, FTO, isocaproic acid, beta-aminoisobutyric acid, butyric acid, allo-isoleucine, tryptophan and glutamine.

11. The method of embodiment 10, wherein the obesity analyte signature comprises a presence of a single nucleotide polymorphism (SNP) or set of SNPs in or near HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, and/or BBS1.

12. The method of any one of the above embodiments, wherein the obesity phenotype of the subject is identified to be abnormal satiation (hungry brain), and wherein the predetermined diet administered to the subject is a volumetrics diet comprising increased dietary fiber.

13. The method of embodiment 12, wherein the increased dietary fiber is represented by a dietary fiber intake of greater than 3.5 g/kcal, wherein g is grams of food.

14. The method of embodiment 12, wherein the increased dietary fiber is represented by a dietary fiber intake of greater than 4 g/kcal, wherein g is grams of food.

15. The method of embodiment 12, wherein the increased dietary fiber is represented by a dietary fiber content above 20 to 45 g/10 MJ or 25 to 45 g/10 MJ, wherein g is grams of food.

16. The method of any one of embodiments 12-15, wherein the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food.

17. The method of any one of embodiments 12-15, wherein the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food.

18. The method of embodiment 12, wherein the increased dietary fiber is as compared to a recommended dietary fiber intake for the subject's demographic group.

19. The method of embodiment 18, wherein the subject is female, and the recommended dietary fiber intake is 21-25 grams of dietary fiber per day.

20. The method of embodiment 18, wherein the subject is a male, and the recommended dietary fiber intake is 30-38 grams of dietary fiber per day.

21. The method of any one of embodiments 12-20, wherein the Caloric content of the volumetric diet comprising increased fiber content is set to 500 Calories less than the basal resting energy expenditure (REE) of the subject.

22. The method of embodiment 21, wherein the REE of the subject is measured by indirect calorimetry.

23. The method of any one of embodiments 12-22, wherein the subject consumes 1-2 meals per day.

24. The method of embodiment 23, wherein the subject is permitted a single serving of a fruit or vegetable in addition to each meal.

25. The method of any one of embodiments 12-24, wherein the intervention responsive obesity analyte signature comprises a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine and ghrelin.

26. The method of any one of embodiments 12-24, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

27. The method of embodiment 26, wherein the intervention responsive obesity analyte signature comprise a presence of a SNP in or near HTR2C, POMC, NPY, AGRP, MC4R, GNB3, SERT, and/or BDNF, and wherein the HADS questionnaire result does not indicate an anxiety sub scale.

28. The method of any one of embodiments 12-27, further comprising administrating an exercise regimen to the subject.

29. The method of embodiment 28, wherein the exercise regimen comprises a combination of resistance training and cardiovascular training for at least 150 minutes per week.

30. The method of any one of embodiments 12-29, further comprising administering one or more pharmacological interventions based on the subject's obesity phenotype.

31. The method of embodiment 30, wherein the one or more pharmacological interventions is phentermine-topiramate pharmacotherapy.

32. The method of any one of embodiments 1-11, wherein the obesity phenotype of the subject is identified to be abnormal satiety (hungry gut), and wherein the predetermined diet administered to the subject is a diet comprising protein supplementation.

33. The method of embodiment 32, wherein the Caloric content of the diet comprising protein supplementation is set to 500 Calories less than the basal resting energy expenditure (REE) of the subject.

34. The method of embodiment 33, wherein the REE of the subject is measured by indirect calorimetry.

35. The method of any one of embodiments 32-34, wherein the subject consumes 3-5 meals per day.

36. The method of any one of embodiments 32-35, wherein the protein supplementation comprises a pre-meal protein shake, and the method further comprises consuming the pre-meal protein shake at least 30 minutes prior to the meal.

37. The method of any one of embodiments 32-35, wherein the protein supplementation comprises a pre-meal protein snack and the method further comprises consuming the pre-meal protein snack at least 60 minutes prior to the meal.

38. The method of any one of embodiments 32-37, wherein the diet comprising protein supplementation comprises greater than 0.8 g/kg of body weight (BW) per day of protein.

39. The method of any one of embodiments 32-37, wherein the diet comprising protein supplementation comprises greater than 20% of total energy per day derived from protein.

40. The method of any one of embodiments 32-37, wherein the diet comprising protein supplementation comprises greater than 46 grams per day of protein if the subject is female or 56 grams per day if the subject is male.

41. The method of any one of embodiments 32-40, wherein the intervention responsive obesity analyte signature comprises a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY.

42. The method of any one of embodiments 32-40, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

43. The method of embodiment 42, wherein the intervention responsive obesity analyte signature comprise a presence of a SNP in or near PYY, GLP-1, MC4R, GPBAR1, TCF7L2, ADAR2A, PCSK, and/or TMEM18.

44. The method of any one of embodiments 32-43, further comprising administrating an exercise regimen to the subject.

45. The method of embodiment 44, wherein the exercise regimen comprises a combination of resistance training and cardiovascular training for at least 150 minutes per week.

46. The method of any one of embodiments 32-45, further comprising administering one or more pharmacological interventions based on the subject's obesity phenotype.

47. The method of embodiment 46, wherein the one or more pharmacological interventions is a long-acting GLP-1 receptor agonist.

48. The method of embodiment 47, wherein said GLP-1 receptor agonist comprises liraglutide or semaglutide.

49. The method of any one of embodiments 1-11, wherein the obesity phenotype of the subject is identified to be abnormal hedonic/behavior (emotional hunger), and wherein the predetermined diet administered to the subject is a low-calorie diet comprising an absence of snacks between meals.

50. The method of embodiment 49, wherein the Caloric content of the low-calorie diet comprising an absence of snacks between meals is set to 500 Calories less than the basal resting energy expenditure (REE) of the subject.

51. The method of embodiment 50, wherein the REE of the subject is measured by indirect calorimetry.

52. The method of any one of embodiments 49-51, wherein the subject consumes 3 meals per day.

53. The method of any one of embodiments 49-52, wherein the intervention responsive obesity analyte signature comprises a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and wherein the HADS questionnaire result indicates a positive in an anxiety subscale.

54. The method of any one of embodiments 49-52, wherein the intervention responsive obesity analyte signature comprises a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin, and wherein the HADS questionnaire result is positive in an anxiety component.

55. The method of any one of embodiments 49-52, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

56. The method of embodiment 55, wherein the intervention responsive obesity analyte signature comprise a presence of a SNP in or near SLC6A4/SERT and/or DRD2 OR a SNP in TCF7L2, UCP3 and/or ADRA2A; wherein the HADS questionnaire result is positive in an anxiety component.

57. The method of any one of embodiments 49-56, further comprising administrating an exercise regimen to the subject.

58. The method of embodiment 57, wherein the exercise regimen comprises a combination of resistance training and cardiovascular training.

59. The method of any one of embodiments 49-58, further comprising subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change.

60. The method of any one of embodiments 49-59, further comprising administering one or more pharmacological interventions based on the subject's obesity phenotype.

61. The method of embodiment 60, wherein the one or more pharmacological interventions is naltrexone-bupropion pharmacotherapy.

62. The method of any one of embodiments 1-11, wherein the obesity phenotype of the subject is identified to be slow metabolism (slow burn), and wherein the predetermined diet administered to the subject is a low-calorie diet comprising post-exercise protein shake or protein snack.

63. The method of embodiment 62, wherein the Caloric content of the low-calorie diet comprising an absence of snacks between meals is set to 500 Calories less than the basal resting energy expenditure (REE) of the subject.

64. The method of embodiment 63, wherein the REE of the subject is measured by indirect calorimetry.

65. The method of any one of embodiments 62-64, wherein the subject consumes 3 meals per day.

66. The method of any one of embodiments 62-65, further comprising consuming the post-exercise protein shake or protein snack at least 30 minutes after ending an exercise session.

67. The method of any one of embodiments 62-66, wherein the intervention responsive obesity analyte signature comprises a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine, and wherein said HADS questionnaire result indicates a positive in an anxiety subscale.

68. The method of any one of embodiments 62-66, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

69. The method of embodiment 68, wherein the intervention responsive obesity analyte signature comprise a presence of a SNP in or near FTO, LEP, LEPR, UCP1, UCP2, UCP3, ADRA2, KLF14, NPC1, LYPLAL1, ADRB2, ADRB3 and/or BBS1; wherein the HADS questionnaire result indicates a positive in an anxiety sub scale.

70. The method of any one of embodiments 62-69, further comprising administrating an exercise regimen to the subject.

71. The method of embodiment 70, wherein the exercise regimen comprises a combination of resistance training and high intensity interval training (HIIT) training.

72. The method of any one of embodiments 62-71, further comprising administering one or more pharmacological interventions based on the subject's obesity phenotype.

73. The method of embodiment 30, wherein the one or more pharmacological interventions is phentermine pharmacotherapy.

74. A method of treating obesity in a subject in need thereof, the method comprising administering a predetermined diet to a subject, wherein the subject has an abnormal satiation (hungry brain) obesity phenotype, and wherein the predetermined diet is a volumetrics diet comprising increased dietary fiber.

75. The method of embodiment 74, wherein the increased dietary fiber is represented by a dietary fiber intake of greater than 3.5 g/kcal, wherein g is grams of food.

76. The method of embodiment 74, wherein the increased dietary fiber is represented by a dietary fiber intake of greater than 4 g/kcal, wherein g is grams of food.

77. The method of embodiment 74, wherein the increased dietary fiber is represented by a dietary fiber content above 20 to 45 g/10 MJ or 25 to 45 g/10 MJ, wherein g is grams of food.

78. The method of any one of embodiments 74-77, wherein the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food.

79. The method of any one of embodiments 74-77, wherein the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food.

80. The method of embodiment 74, wherein the increased dietary fiber is as compared to a recommended dietary fiber intake for the subject's demographic group.

81. The method of embodiment 80, wherein the subject is a female, and the recommended dietary fiber intake is 21-25 grams of dietary fiber per day.

82. The method of embodiment 80, wherein the subject is a male, and the recommended dietary fiber intake is 30-38 grams of dietary fiber per day.

83. The method of any one of embodiments 74-82, wherein the Caloric content of the volumetric diet comprising increased fiber content is set to 500 Calories less than the basal resting energy expenditure (REE) of the subject.

84. The method of embodiment 83, wherein the REE of the subject is measured by indirect calorimetry.

85. The method of any one of embodiments 74-84, wherein the subject consumes 1-2 meals per day.

86. The method of embodiment 85, wherein the subject is permitted a single serving of a fruit or vegetable in addition to each meal.

87. The method of any one of embodiments 74-86, wherein the administration of the predetermined diet is effective to reduce the total body weight of the subject by at least 4%.

88. The method of any one of embodiments 74-86, wherein the administration of the predetermined diet is effective to reduce the total body weight of the subject by 2% to 8%.

89. The method of any one of embodiments 74-88, wherein the administration of the predetermined diet is effective to reduce the waist circumference of the subject by from about 1 inches to about 10 inches.

90. The method of any one of embodiments 74-89, further comprising administrating an exercise regimen to the subject.

91. The method of embodiment 90, wherein the exercise regimen comprises a combination of resistance training and cardiovascular training.

92. The method of any one of embodiments 74-91, further comprising administering one or more pharmacological interventions based on the subject's obesity phenotype.

93. The method of embodiment 92, wherein the one or more pharmacological interventions is phentermine-topiramate pharmacotherapy.

94. The method of any one of embodiments 74-93, wherein the abnormal satiation phenotype is characterized by the subject having a Hospital Anxiety and Depression Scale (HADS) questionnaire result that does not indicate an anxiety subscale in combination with an obesity analyte signature that comprises detection of a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine, ghrelin in a sample obtained from the subject OR a presence of HTR2C, GNB3, FTO, isocaproic acid, beta-aminoisobutyric acid, butyric acid, allo-isoleucine, tryptophan and glutamine AND a SNP in or near HTR2C, POMC, NPY, AGRP, MC4R, GNB3, SERT, and/or BDNF in a sample obtained from the subject.

95. The method of embodiment 94, wherein the SNP is rs1414334.

96. The method of embodiment 94 or 95, wherein the sample is selected from the group consisting of a blood sample, a saliva sample, a urine sample, a breath sample, and a stool sample.

97. A method of treating obesity in a subject in need thereof, the method comprising administering a predetermined diet to a subject, wherein the subject has an abnormal satiety (hungry gut) obesity phenotype, and wherein the predetermined diet is a diet comprising protein supplementation.

98. The method of embodiment 97, wherein the Caloric content of the diet comprising protein supplementation is set to 500 Calories less than the basal resting energy expenditure (REE) of the subject.

99. The method of embodiment 98, wherein the REE of the subject is measured by indirect calorimetry.

100. The method of any one of embodiments 97-99, wherein the subject consumes 3-5 meals per day.

101. The method of any one of embodiments 97-100, wherein the protein supplementation comprises a pre-meal protein shake, and the method further comprises consuming the pre-meal protein shake at least 30 minutes prior to the meal.

102. The method of any one of embodiments 97-100, wherein the protein supplementation comprises a pre-meal protein snack and the method further comprises consuming the pre-meal protein snack at least 60 minutes prior to the meal.

103. The method of any one of embodiments 97-102, wherein the diet comprising protein supplementation comprises greater than 0.8 g/kg of body weight (BW) per day of protein.

104. The method of any one of embodiments 97-102, wherein the diet comprising protein supplementation comprises greater than 20% of total energy per day derived from protein.

105. The method of any one of embodiments 97-102, wherein the diet comprising protein supplementation comprises greater than 46 grams per day of protein if the subject is female or 56 grams per day if the subject is male.

106. The method of any one of embodiments 97-105, wherein the administration of the predetermined diet is effective to reduce the total body weight of the subject by at least 4%.

107. The method of any one of embodiments 97-105, wherein the administration of the predetermined diet is effective to reduce the total body weight of the subject by 2% to 8%.

108. The method of any one of embodiments 97-107, wherein the administration of the predetermined diet is effective to reduce the waist circumference of the subject by from about 1 inches to about 10 inches.

109. The method of any one of embodiments 97-108, further comprising administrating an exercise regimen to the subject.

110. The method of embodiment 109, wherein the exercise regimen comprises a combination of resistance training and cardiovascular training.

111. The method of any one of embodiments 97-110, further comprising administering one or more pharmacological interventions based on the subject's obesity phenotype.

112. The method of embodiment 111, wherein the one or more pharmacological interventions is a GLP-1 receptor agonist.

113. The method of embodiment 112, wherein said GLP-1 receptor agonist comprises liraglutide.

114. The method of any one of embodiments 97-113, wherein the abnormal satiety phenotype is characterized by the subject having a Hospital Anxiety and Depression Scale (HADS) questionnaire result that does not indicate an anxiety subscale in combination with an obesity analyte signature that comprises detection of a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY in a sample obtained from the subject OR a presence of HTR2C, GNB3, FTO, isocaproic acid, beta-aminoisobutyric acid, butyric acid, allo-isoleucine, tryptophan and glutamine AND a SNP in or near PYY, GLP-1, MC4R, GPBAR1, TCF7L2, ADAR2A, PCSK, and/or TMEM18 in a sample obtained from the subject.

115. The method of embodiment 114, wherein the SNP is rs7903146.

116. The method of embodiment 114 or 115, wherein the sample is selected from the group consisting of a blood sample, a saliva sample, a urine sample, a breath sample, and a stool sample.

117. A method of treating obesity in a subject in need thereof, the method comprising administering a predetermined diet to a subject, wherein the subject has an abnormal hedonic/behavior (emotional hunger), and wherein the predetermined diet administered to the subject is a low-calorie diet comprising an absence of snacks between meals.

118. The method of embodiment 117, wherein the Caloric content of the high protein diet comprising pre-meal protein shake or protein snack is set to 500 Calories less than the basal resting energy expenditure (REE) of the subject.

119. The method of embodiment 118, wherein the REE of the subject is measured by indirect calorimetry.

120. The method of any one of embodiments 117-119, wherein the subject consumes 3 meals per day.

121. The method of any one of embodiments 117-120, wherein the administration of the predetermined diet is effective to reduce the total body weight of the subject by at least 4%.

122. The method of any one of embodiments 117-120, wherein the administration of the predetermined diet is effective to reduce the total body weight of the subject by 2% to 8%.

123. The method of any one of embodiments 117-122, wherein the administration of the predetermined diet is effective to reduce the waist circumference of the subject by from about 1 inches to about 10 inches.

124. The method of any one of embodiments 117-123, further comprising administrating an exercise regimen to the subject.

125. The method of embodiment 124, wherein the exercise regimen comprises a combination of resistance training and cardiovascular training.

126. The method of any one of embodiments 117-125, further comprising subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change.

127. The method of any one of embodiments 117-126, further comprising administering one or more pharmacological interventions based on the subject's obesity phenotype.

128. The method of embodiment 127, wherein the one or more pharmacological interventions is a naltrexone-bupropion pharmacotherapy.

129. The method of any one of embodiments 117-128, wherein the abnormal hedonic/behavior phenotype is characterized by the subject having a Hospital Anxiety and Depression Scale (HADS) questionnaire result that indicates an anxiety subscale in combination with an obesity analyte signature that comprises detection of a presence of a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY in a sample obtained from the subject; a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin in a sample obtained from the subject; OR a presence of HTR2C, GNB3, FTO, isocaproic acid, beta-aminoisobutyric acid, butyric acid, allo-isoleucine, tryptophan and glutamine AND a presence of a SNP in or near SLC6A4/SERT and/or DRD2; OR a presence of HTR2C, GNB3, FTO, isocaproic acid, beta-aminoisobutyric acid, butyric acid, allo-isoleucine, tryptophan and glutamine AND a SNP in or near TCF7L2, UCP3 and/or ADRA2A in a sample obtained from the subject.

130. The method of embodiment 129, wherein the SNP is rs4795541 or rs1626521.

131. The method of embodiment 129 or 130, wherein the sample is selected from the group consisting of a blood sample, a saliva sample, a urine sample, a breath sample, and a stool sample.

132. A method of treating obesity in a subject in need thereof, the method comprising administering a predetermined diet to a subject, wherein the subject has a slow metabolism (slow burn) obesity phenotype, and wherein the predetermined diet administered to the subject is a low-calorie diet comprising post-exercise protein shake or protein snack.

133. The method of embodiment 132, wherein the Caloric content of the high protein diet comprising pre-meal protein shake or protein snack is set to 500 Calories less than the basal resting energy expenditure (REE) of the subject.

134. The method of embodiment 133, wherein the REE of the subject is measured by indirect calorimetry.

135. The method of any one of embodiments 132-134, wherein the subject consumes 3 meals per day.

136. The method of any one of embodiments 132-135, further comprising consuming the post-exercise protein shake or protein snack at least 30 minutes after ending an exercise session.

137. The method of any one of claims 132-136, wherein the administration of the predetermined diet is effective to reduce the total body weight of the subject by at least 4%.

138. The method of any one of claims 132-136, wherein the administration of the predetermined diet is effective to reduce the total body weight of the subject by 2% to 8%.

139. The method of any one of claims 132-138, wherein the administration of the predetermined diet is effective to reduce the waist circumference of the subject by from about 1 inches to about 10 inches.

140. The method of any one of claims 132-139, further comprising administrating an exercise regimen to the subject.

141. The method of claim 140, wherein the exercise regimen comprises a combination of resistance training and high intensity interval training (HIIT) training.

142. The method of any one of claims 132-141, further comprising administering one or more pharmacological interventions based on the subject's obesity phenotype.

143. The method of claim 142, wherein the one or more pharmacological interventions is a phentermine pharmacotherapy.

144. The method of any one of claims 132-143, wherein the abnormal satiation phenotype is characterized by the subject having a Hospital Anxiety and Depression Scale (HADS) questionnaire result that indicates an anxiety subscale in combination with an obesity analyte signature that comprises detection of a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine in a sample obtained from the subject OR a presence of HTR2C, GNB3, FTO, isocaproic acid, beta-aminoisobutyric acid, butyric acid, allo-isoleucine, tryptophan and glutamine AND a SNP in or near FTO, LEP, LEPR, UCP1, UCP2, UCP3, ADRA2, KLF14, NPC1, LYPLAL1, ADRB2, ADRB3 and/or BBS1 in a sample obtained from the subject.

145. The method of claim 144, wherein the SNP is rs2075577.

146. The method of claim 144 or 145, wherein the sample is selected from the group consisting of a blood sample, a saliva sample, a urine sample, a breath sample, and a stool sample.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method of treating obesity in a subject in need thereof, the method comprising: (a) identifying an obesity phenotype of a subject by detecting an intervention responsive obesity analyte signature in a sample obtained from a subject; and (b) administering a predetermined lifestyle and behavioral plan to the subject, wherein the predetermined lifestyle and behavioral plan is individualized to the obesity phenotype of the subject, thereby treating obesity in the subject.

2. The method of claim 1, wherein the administration of the predetermined diet results in weight loss, wherein at least part of the weight loss is due to a loss of fat mass.

3. The method of claim 1 or 2, wherein the administration of the predetermined diet is effective to reduce the total body weight of the subject by at least 4%.

4. The method of any one of the above claims, wherein the administration of the predetermined diet is effective to reduce the total body weight of the subject by 2% to 8%.

5. The method of any one of the above claims, wherein the administration of the predetermined diet is effective to reduce the waist circumference of the subject by from about 1 inches to about 10 inches.

6. The method of any one of the above claims, wherein determining the obesity phenotype of the subject further comprises obtaining results from a Hospital Anxiety and Depression Scale (HADS) questionnaire.

7. The method of any one of the above claims, wherein the detecting the intervention responsive obesity analyte signature comprises detecting the presence, absence, or level of one or more metabolites, detecting the presence, absence, or level of one or more peptides, and/or detecting the presence, absence, or level of one or more single nucleotide polymorphisms (SNPs) in the sample obtained from the subject.

8. The method of any one of the above claims, wherein the sample is selected from the group consisting of a blood sample, a saliva sample, a urine sample, a breath sample, and a stool sample.

9. The method of any one of the above claims, wherein the obesity analyte signature comprises 1-methylhistine, serotonin, glutamine, gamma-amino-n-butyric-acid, isocaproic acid, allo-isoleucine, hydroxyproline, beta-aminoisobutyric-acid, alanine, hexanoic acid, tyrosine, ghrelin, peptide tyrosine tyrosine (PYY), GLP-1 and phenylalanine.

10. The method of any one of claims 1-8, wherein the obesity analyte signature comprises a presence of HTR2C, GNB3, FTO, isocaproic acid, beta-aminoisobutyric acid, butyric acid, allo-isoleucine, tryptophan and glutamine.

11. The method of claim 10, wherein the obesity analyte signature comprises a presence of a single nucleotide polymorphism (SNP) or set of SNPs in or near HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, and/or BBS1.

12. The method of any one of the above claims, wherein the obesity phenotype of the subject is identified to be abnormal satiation (hungry brain), and wherein the predetermined diet administered to the subject is a volumetrics diet comprising increased dietary fiber.

13. The method of claim 12, wherein the increased dietary fiber is represented by a dietary fiber intake of greater than 3.5 g/kcal, wherein g is grams of food.

14. The method of claim 12, wherein the increased dietary fiber is represented by a dietary fiber intake of greater than 4 g/kcal, wherein g is grams of food.

15. The method of claim 12, wherein the increased dietary fiber is represented by a dietary fiber content above 20 to 45 g/10 MJ or 25 to 45 g/10 MJ, wherein g is grams of food.

16. The method of any one of claims 12-15, wherein the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food.

17. The method of any one of claims 12-15, wherein the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food.

18. The method of claim 12, wherein the increased dietary fiber is as compared to a recommended dietary fiber intake for the subject's demographic group.

19. The method of claim 18, wherein the subject is female, and the recommended dietary fiber intake is 21-25 grams of dietary fiber per day.

20. The method of claim 18, wherein the subject is a male, and the recommended dietary fiber intake is 30-38 grams of dietary fiber per day.

21. The method of any one of claims 12-20, wherein the Caloric content of the volumetric diet comprising increased fiber content is set to 500 Calories less than the basal resting energy expenditure (REE) of the subject.

22. The method of claim 21, wherein the REE of the subject is measured by indirect calorimetry.

23. The method of any one of claims 12-22, wherein the subject consumes 1-2 meals per day.

24. The method of claim 23, wherein the subject is permitted a single serving of a fruit or vegetable in addition to each meal.

25. The method of any one of claims 12-24, wherein the intervention responsive obesity analyte signature comprises a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine and ghrelin.

26. The method of any one of claims 12-24, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

27. The method of claim 26, wherein the intervention responsive obesity analyte signature comprise a presence of a SNP in or near HTR2C, POMC, NPY, AGRP, MC4R, GNB3, SERT, and/or BDNF, and wherein the HADS questionnaire result does not indicate an anxiety sub scale.

28. The method of any one of claims 12-27, further comprising administrating an exercise regimen to the subject.

29. The method of claim 28, wherein the exercise regimen comprises a combination of resistance training and cardiovascular training for at least 150 minutes per week.

30. The method of any one of claims 12-29, further comprising administering one or more pharmacological interventions based on the subject's obesity phenotype.

31. The method of claim 30, wherein the one or more pharmacological interventions is phentermine-topiramate pharmacotherapy.

32. The method of any one of claims 1-11, wherein the obesity phenotype of the subject is identified to be abnormal satiety (hungry gut), and wherein the predetermined diet administered to the subject is a diet comprising protein supplementation.

33. The method of claim 32, wherein the Caloric content of the diet comprising protein supplementation is set to 500 Calories less than the basal resting energy expenditure (REE) of the subject.

34. The method of claim 33, wherein the REE of the subject is measured by indirect calorimetry.

35. The method of any one of claims 32-34, wherein the subject consumes 3-5 meals per day.

36. The method of any one of claims 32-35, wherein the protein supplementation comprises a pre-meal protein shake, and the method further comprises consuming the pre-meal protein shake at least 30 minutes prior to the meal.

37. The method of any one of claims 32-35, wherein the protein supplementation comprises a pre-meal protein snack and the method further comprises consuming the pre-meal protein snack at least 60 minutes prior to the meal.

38. The method of any one of claims 32-37, wherein the diet comprising protein supplementation comprises greater than 0.8 g/kg of body weight (BW) per day of protein.

39. The method of any one of claims 32-37, wherein the diet comprising protein supplementation comprises greater than 20% of total energy per day derived from protein.

40. The method of any one of claims 32-37, wherein the diet comprising protein supplementation comprises greater than 46 grams per day of protein if the subject is female or 56 grams per day if the subject is male.

41. The method of any one of claims 32-40, wherein the intervention responsive obesity analyte signature comprises a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY.

42. The method of any one of claims 32-40, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

43. The method of claim 42, wherein the intervention responsive obesity analyte signature comprise a presence of a SNP in or near PYY, GLP-1, MC4R, GPBAR1, TCF7L2, ADAR2A, PCSK, and/or TMEM18.

44. The method of any one of claims 32-43, further comprising administrating an exercise regimen to the subject.

45. The method of claim 44, wherein the exercise regimen comprises a combination of resistance training and cardiovascular training for at least 150 minutes per week.

46. The method of any one of claims 32-45, further comprising administering one or more pharmacological interventions based on the subject's obesity phenotype.

47. The method of claim 46, wherein the one or more pharmacological interventions is a long-acting GLP-1 receptor agonist.

48. The method of claim 47, wherein said GLP-1 receptor agonist comprises liraglutide or semaglutide.

49. The method of any one of claims 1-11, wherein the obesity phenotype of the subject is identified to be abnormal hedonic/behavior (emotional hunger), and wherein the predetermined diet administered to the subject is a low-calorie diet comprising an absence of snacks between meals.

50. The method of claim 49, wherein the Caloric content of the low-calorie diet comprising an absence of snacks between meals is set to 500 Calories less than the basal resting energy expenditure (REE) of the subject.

51. The method of claim 50, wherein the REE of the subject is measured by indirect calorimetry.

52. The method of any one of claims 49-51, wherein the subject consumes 3 meals per day.

53. The method of any one of claims 49-52, wherein the intervention responsive obesity analyte signature comprises a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY, and wherein the HADS questionnaire result indicates a positive in an anxiety subscale.

54. The method of any one of claims 49-52, wherein the intervention responsive obesity analyte signature comprises a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin, and wherein the HADS questionnaire result is positive in an anxiety component.

55. The method of any one of claims 49-52, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

56. The method of claim 55, wherein the intervention responsive obesity analyte signature comprise a presence of a SNP in or near SLC6A4/SERT and/or DRD2 OR a SNP in TCF7L2, UCP3 and/or ADRA2A; wherein the HADS questionnaire result is positive in an anxiety component.

57. The method of any one of claims 49-56, further comprising administrating an exercise regimen to the subject.

58. The method of claim 57, wherein the exercise regimen comprises a combination of resistance training and cardiovascular training.

59. The method of any one of claims 49-58, further comprising subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change.

60. The method of any one of claims 49-59, further comprising administering one or more pharmacological interventions based on the subject's obesity phenotype.

61. The method of claim 60, wherein the one or more pharmacological interventions is naltrexone-bupropion pharmacotherapy.

62. The method of any one of claims 1-11, wherein the obesity phenotype of the subject is identified to be slow metabolism (slow burn), and wherein the predetermined diet administered to the subject is a low-calorie diet comprising post-exercise protein shake or protein snack.

63. The method of claim 62, wherein the Caloric content of the low-calorie diet comprising an absence of snacks between meals is set to 500 Calories less than the basal resting energy expenditure (REE) of the subject.

64. The method of claim 63, wherein the REE of the subject is measured by indirect calorimetry.

65. The method of any one of claims 62-64, wherein the subject consumes 3 meals per day.

66. The method of any one of claims 62-65, further comprising consuming the post-exercise protein shake or protein snack at least 30 minutes after ending an exercise session.

67. The method of any one of claims 62-66, wherein the intervention responsive obesity analyte signature comprises a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine, and wherein said HADS questionnaire result indicates a positive in an anxiety subscale.

68. The method of any one of claims 62-66, wherein the intervention responsive obesity analyte signature comprises a presence of HTR2C, GNB3, FTO, isocaproic acid, beta-aminoisobutyric acid, butyric acid, allo-isoleucine, tryptophan and glutamine.

69. The method of claim 68, wherein the intervention responsive obesity analyte signature comprise a presence of a SNP in or near FTO, LEP, LEPR, UCP1, UCP2, UCP3, ADRA2, KLF14, NPC1, LYPLAL1, ADRB2, ADRB3 and/or BBS1; wherein the HADS questionnaire result indicates a positive in an anxiety sub scale.

70. The method of any one of claims 62-69, further comprising administrating an exercise regimen to the subject.

71. The method of claim 70, wherein the exercise regimen comprises a combination of resistance training and high intensity interval training (HIIT) training.

72. The method of any one of claims 62-71, further comprising administering one or more pharmacological interventions based on the subject's obesity phenotype.

73. The method of claim 30, wherein the one or more pharmacological interventions is phentermine pharmacotherapy.

74. A method of treating obesity in a subject in need thereof, the method comprising administering a predetermined diet to a subject, wherein the subject has an abnormal satiation (hungry brain) obesity phenotype, and wherein the predetermined diet is a volumetrics diet comprising increased dietary fiber.

75. The method of claim 74, wherein the increased dietary fiber is represented by a dietary fiber intake of greater than 3.5 g/kcal, wherein g is grams of food.

76. The method of claim 74, wherein the increased dietary fiber is represented by a dietary fiber intake of greater than 4 g/kcal, wherein g is grams of food.

77. The method of claim 74, wherein the increased dietary fiber is represented by a dietary fiber content above 20 to 45 g/10 MJ or 25 to 45 g/10 MJ, wherein g is grams of food.

78. The method of any one of claims 74-77, wherein the energy density of the volumetrics diet is less than 115 kcal/100 g, less than 112 kcal/100 g, less than 110 kcal/100 g, less than 105 kcal/100 g, less than 100 kcal/100 g, less than 95 kcal/100 g or less than 90 kcal/100 g, wherein g is grams of food.

79. The method of any one of claims 74-77, wherein the energy density of the volumetrics diet is between 90 and 115 kcal/100 g, between 90 and 112 kcal/100 g, between 90 and 110 kcal/100 g, between 90 and 105 kcal/100 g or between 90 and 100 kcal/100 g, wherein g is grams of food.

80. The method of claim 74, wherein the increased dietary fiber is as compared to a recommended dietary fiber intake for the subject's demographic group.

81. The method of claim 80, wherein the subject is a female, and the recommended dietary fiber intake is 21-25 grams of dietary fiber per day.

82. The method of claim 80, wherein the subject is a male, and the recommended dietary fiber intake is 30-38 grams of dietary fiber per day.

83. The method of any one of claims 74-82, wherein the Caloric content of the volumetric diet comprising increased fiber content is set to 500 Calories less than the basal resting energy expenditure (REE) of the subject.

84. The method of claim 83, wherein the REE of the subject is measured by indirect calorimetry.

85. The method of any one of claims 74-84, wherein the subject consumes 1-2 meals per day.

86. The method of claim 85, wherein the subject is permitted a single serving of a fruit or vegetable in addition to each meal.

87. The method of any one of claims 74-86, wherein the administration of the predetermined diet is effective to reduce the total body weight of the subject by at least 4%.

88. The method of any one of claims 74-86, wherein the administration of the predetermined diet is effective to reduce the total body weight of the subject by 2% to 8%.

89. The method of any one of claims 74-88, wherein the administration of the predetermined diet is effective to reduce the waist circumference of the subject by from about 1 inches to about 10 inches.

90. The method of any one of claims 74-89, further comprising administrating an exercise regimen to the subject.

91. The method of claim 90, wherein the exercise regimen comprises a combination of resistance training and cardiovascular training.

92. The method of any one of claims 74-91, further comprising administering one or more pharmacological interventions based on the subject's obesity phenotype.

93. The method of claim 92, wherein the one or more pharmacological interventions is phentermine-topiramate pharmacotherapy.

94. The method of any one of claims 74-93, wherein the abnormal satiation phenotype is characterized by the subject having a Hospital Anxiety and Depression Scale (HADS) questionnaire result that does not indicate an anxiety subscale in combination with an obesity analyte signature that comprises detection of a presence of serotonin, glutamine, isocaproic acid, allo-isoleucine, hydroxyproline, beta-aminoisobutyric-acid, alanine, hexanoic acid, tyrosine, and PYY, and an absence of 1-methylhistine, gamma-amino-n-butyric-acid, phenylalanine, ghrelin in a sample obtained from the subject OR a presence of HTR2C, GNB3, FTO, isocaproic acid, beta-aminoisobutyric acid, butyric acid, allo-isoleucine, tryptophan and glutamine AND a SNP in or near HTR2C, POMC, NPY, AGRP, MC4R, GNB3, SERT, and/or BDNF in a sample obtained from the subject.

95. The method of claim 94, wherein the SNP is rs1414334.

96. The method of claim 94 or 95, wherein the sample is selected from the group consisting of a blood sample, a saliva sample, a urine sample, a breath sample, and a stool sample.

97. A method of treating obesity in a subject in need thereof, the method comprising administering a predetermined diet to a subject, wherein the subject has an abnormal satiety (hungry gut) obesity phenotype, and wherein the predetermined diet is a diet comprising protein supplementation.

98. The method of claim 97, wherein the Caloric content of the diet comprising protein supplementation is set to 500 Calories less than the basal resting energy expenditure (REE) of the subject.

99. The method of claim 98, wherein the REE of the subject is measured by indirect calorimetry.

100. The method of any one of claims 97-99, wherein the subject consumes 3-5 meals per day.

101. The method of any one of claims 97-100, wherein the protein supplementation comprises a pre-meal protein shake, and the method further comprises consuming the pre-meal protein shake at least 30 minutes prior to the meal.

102. The method of any one of claims 97-100, wherein the protein supplementation comprises a pre-meal protein snack and the method further comprises consuming the pre-meal protein snack at least 60 minutes prior to the meal.

103. The method of any one of claims 97-102, wherein the diet comprising protein supplementation comprises greater than 0.8 g/kg of body weight (BW) per day of protein.

104. The method of any one of claims 97-102, wherein the diet comprising protein supplementation comprises greater than 20% of total energy per day derived from protein.

105. The method of any one of claims 97-102, wherein the diet comprising protein supplementation comprises greater than 46 grams per day of protein if the subject is female or 56 grams per day if the subject is male.

106. The method of any one of claims 97-105, wherein the administration of the predetermined diet is effective to reduce the total body weight of the subject by at least 4%.

107. The method of any one of claims 97-105, wherein the administration of the predetermined diet is effective to reduce the total body weight of the subject by 2% to 8%.

108. The method of any one of claims 97-107, wherein the administration of the predetermined diet is effective to reduce the waist circumference of the subject by from about 1 inches to about 10 inches.

109. The method of any one of claims 97-108, further comprising administrating an exercise regimen to the subject.

110. The method of claim 109, wherein the exercise regimen comprises a combination of resistance training and cardiovascular training.

111. The method of any one of claims 97-110, further comprising administering one or more pharmacological interventions based on the subject's obesity phenotype.

112. The method of claim 111, wherein the one or more pharmacological interventions is a GLP-1 receptor agonist.

113. The method of claim 112, wherein said GLP-1 receptor agonist comprises liraglutide.

114. The method of any one of claims 97-113, wherein the abnormal satiety phenotype is characterized by the subject having a Hospital Anxiety and Depression Scale (HADS) questionnaire result that does not indicate an anxiety subscale in combination with an obesity analyte signature that comprises detection of a presence of 1-methylhistine, allo-isoleucine, hydroxyproline, beta-aminoisobutyric-acid, alanine, and phenylalanine, and an absence of serotonin, glutamine, gamma-amino-n-butyric-acid, isocaproic acid, hexanoic acid, tyrosine, ghrelin, and PYY in a sample obtained from the subject OR a presence of HTR2C, GNB3, FTO, isocaproic acid, beta-aminoisobutyric acid, butyric acid, allo-isoleucine, tryptophan and glutamine AND a SNP in or near PYY, GLP-1, MC4R, GPBAR1, TCF7L2, ADAR2A, PCSK, and/or TMEM18 in a sample obtained from the subject.

115. The method of claim 114, wherein the SNP is rs7903146.

116. The method of claim 114 or 115, wherein the sample is selected from the group consisting of a blood sample, a saliva sample, a urine sample, a breath sample, and a stool sample.

117. A method of treating obesity in a subject in need thereof, the method comprising administering a predetermined diet to a subject, wherein the subject has an abnormal hedonic/behavior (emotional hunger), and wherein the predetermined diet administered to the subject is a low-calorie diet comprising an absence of snacks between meals.

118. The method of claim 117, wherein the Caloric content of the high protein diet comprising pre-meal protein shake or protein snack is set to 500 Calories less than the basal resting energy expenditure (REE) of the subject.

119. The method of claim 118, wherein the REE of the subject is measured by indirect calorimetry.

120. The method of any one of claims 117-119, wherein the subject consumes 3 meals per day.

121. The method of any one of claims 117-120, wherein the administration of the predetermined diet is effective to reduce the total body weight of the subject by at least 4%.

122. The method of any one of claims 117-120, wherein the administration of the predetermined diet is effective to reduce the total body weight of the subject by 2% to 8%.

123. The method of any one of claims 117-122, wherein the administration of the predetermined diet is effective to reduce the waist circumference of the subject by from about 1 inches to about 10 inches.

124. The method of any one of claims 117-123, further comprising administrating an exercise regimen to the subject.

125. The method of claim 124, wherein the exercise regimen comprises a combination of resistance training and cardiovascular training.

126. The method of any one of claims 117-125, further comprising subjecting the subject to an emotional eating group comprising sessions covering goal setting, self-monitoring, triggers for overeating, thoughts, feelings and behaviors, resiliency and positive coping strategies, emotional regulation, mindfulness and mindful eating, body positivity and self-compassion, support network, high-risk eating situations, behavior chain and maintaining motivation for change.

127. The method of any one of claims 117-126, further comprising administering one or more pharmacological interventions based on the subject's obesity phenotype.

128. The method of claim 127, wherein the one or more pharmacological interventions is a naltrexone-bupropion pharmacotherapy.

129. The method of any one of claims 117-128, wherein the abnormal hedonic/behavior phenotype is characterized by the subject having a Hospital Anxiety and Depression Scale (HADS) questionnaire result that indicates an anxiety subscale in combination with an obesity analyte signature that comprises detection of a presence of a presence of serotonin, and an absence of 1-methylhistine, glutamine, gamma-amino-n-butyric-acid, isocaproic acid, allo-isoleucine, hydroxyproline, beta-aminoisobutyric-acid, alanine, hexanoic acid, tyrosine, phenylalanine, ghrelin, and PYY in a sample obtained from the subject; a presence of 1-methylhistine, glutamine, gamma-amino-n-butyric-acid, isocaproic acid, allo-isoleucine, beta-aminoisobutyric-acid, alanine, hexanoic acid, tyrosine, phenylalanine, PYY, and an absence of serotonin, hydroxyproline, and ghrelin in a sample obtained from the subject; OR a presence of HTR2C, GNB3, FTO, isocaproic acid, beta-aminoisobutyric acid, butyric acid, allo-isoleucine, tryptophan and glutamine AND a presence of a SNP in or near SLC6A4/SERT and/or DRD2; OR a presence of HTR2C, GNB3, FTO, isocaproic acid, beta-aminoisobutyric acid, butyric acid, allo-isoleucine, tryptophan and glutamine AND a SNP in or near TCF7L2, UCP3 and/or ADRA2A in a sample obtained from the subject.

130. The method of claim 129, wherein the SNP is rs4795541 or rs1626521.

131. The method of claim 129 or 130, wherein the sample is selected from the group consisting of a blood sample, a saliva sample, a urine sample, a breath sample, and a stool sample.

132. A method of treating obesity in a subject in need thereof, the method comprising administering a predetermined diet to a subject, wherein the subject has a slow metabolism (slow burn) obesity phenotype, and wherein the predetermined diet administered to the subject is a low-calorie diet comprising post-exercise protein shake or protein snack.

133. The method of claim 132, wherein the Caloric content of the high protein diet comprising pre-meal protein shake or protein snack is set to 500 Calories less than the basal resting energy expenditure (REE) of the subject.

134. The method of claim 133, wherein the REE of the subject is measured by indirect calorimetry.

135. The method of any one of claims 132-134, wherein the subject consumes 3 meals per day.

136. The method of any one of claims 132-135, further comprising consuming the post-exercise protein shake or protein snack at least 30 minutes after ending an exercise session.

137. The method of any one of claims 132-136, wherein the administration of the predetermined diet is effective to reduce the total body weight of the subject by at least 4%.

138. The method of any one of claims 132-136, wherein the administration of the predetermined diet is effective to reduce the total body weight of the subject by 2% to 8%.

139. The method of any one of claims 132-138, wherein the administration of the predetermined diet is effective to reduce the waist circumference of the subject by from about 1 inches to about 10 inches.

140. The method of any one of claims 132-139, further comprising administrating an exercise regimen to the subject.

141. The method of claim 140, wherein the exercise regimen comprises a combination of resistance training and high intensity interval training (HIIT) training.

142. The method of any one of claims 132-141, further comprising administering one or more pharmacological interventions based on the subject's obesity phenotype.

143. The method of claim 142, wherein the one or more pharmacological interventions is a phentermine pharmacotherapy.

144. The method of any one of claims 132-143, wherein the abnormal satiation phenotype is characterized by the subject having a Hospital Anxiety and Depression Scale (HADS) questionnaire result that indicates an anxiety subscale in combination with an obesity analyte signature that comprises detection of a presence of 1-methylhistine, serotonin, glutamine, gamma-amino-n-butyric-acid, isocaproic acid, allo-isoleucine, alanine, tyrosine, ghrelin, PYY, and an absence of hydroxyproline, beta-aminoisobutyric-acid, hexanoic acid, and phenylalanine in a sample obtained from the subject OR a presence of HTR2C, GNB3, FTO, isocaproic acid, beta-aminoisobutyric acid, butyric acid, allo-isoleucine, tryptophan and glutamine AND a SNP in or near FTO, LEP, LEPR, UCP1, UCP2, UCP3, ADRA2, KLF14, NPC1, LYPLAL1, ADRB2, ADRB3 and/or BBS1 in a sample obtained from the subject.

145. The method of claim 144, wherein the SNP is rs2075577.

146. The method of claim 144 or 145, wherein the sample is selected from the group consisting of a blood sample, a saliva sample, a urine sample, a breath sample, and a stool sample.
